(19) United States
(12) Patent Application Publication
(10) Pub. No.: US 2023/0357773 A1
(43) Pub. Date: Nov. 9, 2023

(54) OLIGONUCLEOTIDE TREATMENT OF HEPATITIS B PATIENTS

(71) Applicants: HOFFMANN-LA ROCHE INC., Little Falls, NJ (US); DICERNA PHARMACEUTICALS, INC., Lexington, MA (US)

(72) Inventors: Søren OTTOSEN, Hørsholm (DK); Henrik MUELLER, Basel (CH); Hardean ACHNECK, Quincy, MA (US); Douglas M. FAMBROUGH, Boston, MA (US); Bob Dale BROWN, Littleton, MA (US)

(21) Appl. No.: 18/164,151

(22) Filed: Feb. 3, 2023

Related U.S. Application Data
(63) Continuation of application No. PCT/EP2021/071824, filed on Aug. 5, 2021.
(60) Provisional application No. 63/061,384, filed on Aug. 5, 2020.

(51) Int. Cl. C12N 15/113; A61P 31/20; A61K 45/06; A61K 31/7088

(52) U.S. Cl. CPC C12N 15/1131; A61P 31/20; A61K 45/06; A61K 31/7088; C12N 2310/11; C12N 2310/322; C12N 2310/321; C12N 2310/315; C12N 2310/3519; C12N 2310/312; C12N 2320/32; C12N 2320/35; C12N 2320/31

(57) ABSTRACT

The present invention provides oligonucleotides for use in the treatment of hepatitis B or hepatitis B virus infection in a human patient.

### TECHNICAL FIELD OF THE INVENTION

The present invention relates to the use of oligonucleotides in methods for treating Hepatitis B or Hepatitis B Virus (HBV) infection in a human patient, particularly uses relating to the treatment of hepatitis B infection in NUC-naïve patients (NUC refers to Nucleos(t)ide Analogue Compounds).

### REFERENCE TO THE SEQUENCE LISTING

The disclosure is being filed along with a Sequence Listing in electronic format. The Sequence Listing is provided as a file entitled P120786PCT_ST25, created on Aug. 1, 2021. The information in electronic format of the Sequence Listing is incorporated herein by reference in its entirety.

### BACKGROUND OF THE INVENTION

Hepatitis B virus (HBV) is a DNA virus that infects hepatocytes and establishes cccDNA (also referred to as a mini-chromosome) within an infected cell that acts as a template for HBV replication and antigenic production. HBV is a hepatotropic, non-cytopathic virus that can cause acute or chronic hepatitis, cirrhosis and/or hepatocellular carcinoma. It is estimated by the World Health Organization that more than two billion people have been infected worldwide, with about 4 million acute cases per year. Sadly, 1 million deaths occur per year and there are 350-400 million chronic carriers. Approximately 25% of carriers die from chronic hepatitis, with progression to cirrhosis occurring at an annual rate of 2-5%, with a yearly incidence of hepatic decompensation of about 3%. For others, cirrhosis or liver cancer occurs in significant numbers while nearly 75% of chronic carriers are Asian. From a global perspective HBV is the second most significant carcinogen behind tobacco, causing from 60% to 80% of all primary liver cancer. At the present time there is a need for effective therapeutic agents for treating humans infected with HBV. In this instance, an effective treatment may include a seroconversion event in the host body in reaction to the virus where there is a 90% or more reduction of viral antigen seen, compared to baseline numbers before treatment, in persons suffering from HBV infection. This type of seroconversion can lead to an effective cure or management of the virus.

Chronic hepatitis B is defined as persistence of hepatitis B surface antigen (HBsAg) in the serum beyond 6 months after acute infection with HBV. Currently, the recommended therapies for chronic HBV infection by the American Association for the Study of Liver Diseases (AASLD) and the European Association for the study of the Liver (EASL) include interferon alpha and pegylated interferon alpha-2a, entecavir and tenofovir respectively (both Nucleos(t)ide Analogue Compounds, NUCs). A key event in the evolution of chronic HBV infection is HBeAg seroconversion that occurs spontaneously at a rate of about 5-10% per year. Under treatment with pegylated interferon, which lasts forty-eight (48) weeks and results in serious and unpleasant side effects, the actual seroconversion (HBeAg seroconversion) events in the twenty-four (24) weeks after therapy has ceased, ranges from only 27-36%. Seroconversion of HBsAg is even lower—only 3% observed immediately after treatment ceases, with an increase to upwards of 12% after 5 years. Current HBV therapies, such as the use of NUCs to suppress the disease may require lifelong therapy to reduce plasma viremia, and they are generally ineffective in the long term. There is a need for improved treatments for Hepatitis B, HBV infection in a human patient and chronic HBV.

The NUC therapies entecavir and tenofovir may successfully reduce viral load in some patients, but the rates of HBeAg seroconversion and of HBsAg loss are even lower than those obtained using interferon therapy. Other similar therapies, including lamivudine (3TC), telbivudine (LdT), and adefovir are also used, but for nucleoside/nucleotide therapies in general, the emergence of resistance limits therapeutic efficacy. In most cases, RNAi therapy has not been considered or tested in patients who are naïve to other drugs for the treatment of chronic HBV infection. The few studies published that test RNAi as part of a first therapy or monotherapy in patients naïve to prior treatment have not revealed flares—which are substantial increases in alanine aminotransferase levels over a defined period of time—that are ‘positive flares’ (“PHBV” i.e. suggestive of an effective host immune-mediated response with maintained normal synthetic and excretory liver function). Previous work has indicated that the use of a combination of RNAi oligonucleotides targeting multiple different HBV genes (namely, S, C, P, and X genes), or in some cases targeting X gene transcripts alone, achieves effective inhibition of HBV replication and gene expression.

Current NUC therapies suppress viral replication, but do not successfully eliminate HBV covalently closed circular DNA (cccDNA), thus leading to HBV rebound and can lead to development of negative hepatic flares upon withdrawal of NUC treatment (Honer et al.,). Pursuant to the current understanding of negative flares, or Negative HBV flares (“NHBV”) these are flares associated with a non-effective immune response that may include declining liver function. Negative flares are typically seen upon a viral “breakthrough” where viral DNA is accumulating and the virus itself is becoming more prevalent in host tissues. This may be seen in the case of a drug resistant breakthrough associated with viral variants in the setting of NUC-treatment, NUC-treatment removal or secondary to Idiosyncratic Drug Toxicity. NHBV flares can also occur while under treatment. These occur due to the recurrence and increase of HBV replication and are typically preceded by an increase of at least 1 log HBVDNA. NHBV flares are considered detrimental events that seldom, if ever, lead to a positive treatment response. In NHBV flare cases, the HBV DNA level may be increasing, or at least not declining and where the underlying therapeutic compound or regimen may have lost its efficacy. Such negative flares or NHBV flares can be life threatening. In these situations, HBV DNA level, or ‘viral load’, is an indicator of viral replication. Higher HBV DNA levels are usually associated with an increased risk of liver disease and hepatocellular carcinoma. HBV DNA level typically fall in response to an effective antiviral treatment. Previous approved HBV therapies for the treatment of chronic HBV are NUCs or enhancements of the host immune response, e.g., IFNs, which have a different mechanism of action than RNAi therapies and rarely produce beneficial flares in the case of NUCs or are fraught with potentially severe side effects in the case of IFNs. As provided above, given the possibility of NHBV flares, NUC therapy may be life long, creating a great financial burden for patients and national health systems. In these discussions the concerns surrounding potential drug toxicity and the selection of mutants that could possibly escape prophylactic vaccination are also important considerations.

In an investigator-initiated cohort study within the European network of excellence for Vigilance against Viral Resistance (VIRGIL) of 729 patients treated with entecavir for their chronic hepatitis B, only 30 patients developed a flare with a cumulative incidence of 6.3% at year 5. Of these flares, only 12 were ‘host induced.’ The authors explained that ‘With host-induced flares, it was assumed that the flare was associated with declining viral load that may lead to a restored immune activity.” (Hepatology ‘Flares during long-term entecavir therapy in chronic hepatitis B, Heng Chi et al, 2016). Therefore, the incidence of ‘beneficial flares’ or positive flares is in the range of approximately 1% per year in NUC-treated patients.

It is now established in the literature that host immune response plays a major role in the outcome of HBV infection. During acute HBV infection, the development of a strong cellular immune response, directed to multiple viral antigens (“Ags”), is associated with the resolution of HBV infection and life-long antiviral immunity.

Thus, there is a significant need in the art to discover and develop new anti-viral therapies. There is a need for therapies with defined doses and dosage regimens that enable improved treatment of hepatitis B or HBV infection with advantageous therapeutic effects over shorter and longer periods of time in patients. More particularly there is a need for new anti-HBV therapies capable of increasing HBeAg and/or HBsAg positive seroconversion rates. These serum markers are indicative of the re-emergence of effective immune system activity relative to the virus and the consequent immunological control of HBV infection that can lead to an improved patient outcome, positive flare or PHBV. Most advantageously, it would be beneficial to find a monotherapy that could provide for a PHBV in an HBV patient and/or increase the number of PHBVs in a patient group. A monotherapy would limit the time of affliction and the reliance on the functioning of multiple modes of action or synergistic effects that may not occur in all patients. Such PHBV flares can be beneficial to the patient if they represent an effective host immune response that leads to a reduction in HBeAg and/or HBsAg, reduced viral load, or to seroclearance of HBV DNA—especially if patients are maintaining the liver's synthetic and excretory function.

It should be noted that PHBVs are the type of flare that is defined as an abrupt increase of alanine aminotransferase (ALT) levels during chronic hepatitis B virus (HBV) infection where the immune response native in the patient has asserted itself or ‘re-emerged’ in the form of a cytotoxic T lymphocyte mediated immune response against HBV where ALT is released from infected hepatocytes in the course of attack by T-cells. Aspects of the current invention provide for uses of oligonucleotides in methods for treating Hepatitis B or HBV infection in human patient. In some embodiments, the disclosure relates to the development of potent oligonucleotides that produce a durable knockdown of HBV surface antigen (HBsAg) expression.

### SUMMARY OF THE INVENTION

According to a first aspect of the invention, there is provided an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP);
or a pharmaceutically acceptable salt thereof,
   for use in a method for treating hepatitis B or hepatitis B virus (HBV) infection in a human patient, said method comprising administering to the patient via the subcutaneous route an initial dose of from about 0.1 mg/kg to about 12 mg/kg of the oligonucleotide.

According to a second aspect of the invention, there is provided an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP);
or a pharmaceutically acceptable salt thereof,
   for use in a method for treating hepatitis B or hepatitis B virus (HBV) infection in a human patient, said method comprising administering to the patient via the subcutaneous route an initial dose of from about 6 mg to about 800 mg of the oligonucleotide.

According to a third aspect of the invention, there is provided a method for treating hepatitis B or hepatitis B virus (HBV) infection in a human patient, the method comprising administering to the patient an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP),
or a pharmaceutically acceptable salt thereof;
   the method comprising administering to the patient via subcutaneous route an initial dose of from about 0.1 mg/kg to about 12 mg/kg of the oligonucleotide.

According to a fourth aspect of the invention, there is provided a method for treating hepatitis B or hepatitis B virus (HBV) infection in a human patient, the method comprising administering to the patient an oligonucleotide comprising a sense strand forming a duplex region with an antisense strand, wherein:
the sense strand consists of a sequence as set forth in
GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP),
or a pharmaceutically acceptable salt thereof;
   the method comprising administering to the patient via subcutaneous route an initial dose of from about 6 mg to about 800 mg of the oligonucleotide.

According to a fifth aspect of the invention, there is provided a use of an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP),
or a pharmaceutically acceptable salt thereof;
   in the manufacture of a medicament for the treatment hepatitis B or hepatitis B virus (HBV) infection in a human patient, comprising administering to the patient via the subcutaneous route an initial dose of from about 0.1 mg/kg to about 12 mg/kg of the oligonucleotide.

According to a sixth aspect of the invention, there is provided a use of an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 4′-carbon of the sugar of the 5′-nucleotide of the antisense strand comprises a methoxy phosphonate (MOP),
or a pharmaceutically acceptable salt thereof;
   in the manufacture of a medicament for the treatment hepatitis B or hepatitis B virus (HBV) infection in a human patient, comprising administering to the patient via the subcutaneous route an initial dose of from about 6 mg to about 800 mg of the oligonucleotide.

The oligonucleotides for use herein are RNAi oligonucleotides.

The invention is based, in part, on the discovery and development of oligonucleotides and pharmaceutically acceptable salts thereof that selectively inhibit and/or reduce HBV expression for extended periods of time and may cause or initiate positive flares/PHBVs in patients. The dosages and dosage regimens of the invention enable improved treatment of hepatitis B and hepatitis B virus HBV infection in human patients and sub patient groups.

The current invention provides a method of monotherapy treatment that can induce PHBV flares, particularly in NUC-naïve patients where HBsAg and HBV DNA are reduced, and excretory liver function is maintained. This is in turn indicative of a positive re-emergence of the host immune system leading to better therapeutic outcomes that may include immunity.

The oligonucleotide provided herein is designed to target an expansive set of HBsAg transcripts encoded by HBV genomes across all known genotypes. It has been found that oligonucleotide disclosed herein can produce a stable reduction in HBsAg expression with high specificity that persists for an extended period of time (e.g., greater than 7 weeks) following administration to a subject.

According to the current invention it is demonstrated that use of a single RNAi oligonucleotide targeting HBsAg transcripts alone also achieves effective inhibition of HBV replication and preferably over extended periods of time and can induce a PHBV, which provides a new therapeutic approach to treating HBV infections and may open the door for advantageous treatment dosages and dosage regimens that have increased and lasting positive outcomes for HBV patients.

The use of the oligonucleotide of the invention may be a monotherapy to treat an HBV infection. Going further, the method of the current invention may also reduce the cost associated with HBV therapy generally while increasing overall safety and tolerability because it would eliminate the potential adverse reactions possibly caused by other (concomitant) or the need for combination therapies. Likewise, a monotherapy approach with the oligonucleotide of the current invention, or even a monotherapy run-in phase prior to a combination therapy, would be highly practical because it would only require infrequent administration of the RNAi oligonucleotide of the invention.

Further, the method of a monotherapy or monotherapy run-in phase prior to a combination therapy may be more efficacious in achieving a functional or sterilizing cure in patients than the immediate treatment with a combination therapy. The reason why a monotherapy or monotherapy run-in phase could be more efficacious is because it may lower the host HBsAg burden sufficiently for the host immune system to clear the hepatitis B virus without additional therapies, or enable to addition of other drugs to effectively clear the virus once the HBsAg level has been reduced and the innate immune system becomes active.

Oligonucleotides for use according to the invention may be provided as combination therapies where multiple modes of action are present and would be beneficial when administered stepwise or simultaneously. For example, previous approved therapies for the treatment of chronic HBV include are oral nucleot(s)ide reverse transcriptase inhibitors (NUCs) or enhancements of the host immune response, (e.g., interferons) each of which have a different mechanism of action than RNAi therapies.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the oligonucleotides for use and methods of treatment disclosed herein.

FIG. 1 shows an example of an HBsAg-targeting oligonucleotide (HBVS-219) with chemical modifications and in duplex form. Darker shade indicates 2′-O-methyl ribonucleotide. Lighter shade indicates 2′-fluoro-deoxyribonucleotide.

FIGS. 2a-2b show the chemical structure of HBVS-219 and HBVS-219P2. FIG. 2a shows the chemical structure for HBVS-219. FIG. 2b shows the chemical structure for HBVS-219P2.

FIG. 3 shows the location of the oligonucleotide target site in the HBV genome (indicated by large X).

FIG. 4 shows mean changes in HBsAg from the baseline (CBL) of treatment for NUC-positive patients (group C). NUC-positive patients were given up to 4 rounds of HBVS-219 on days 1, 29, 57 and 85. Black dashed line (n=6) are placebos from cohorts C1, C2 and C3, light grey solid line (n=4) are HBVS-219 1.5 mg/kg treated cohort C1, medium grey solid line (n=4) are HBVS-219 3 mg/kg treated cohort C2, black solid line (n=3) are HBVS-219 6 mg/kg treated cohort C3 (all doses were per round).

FIG. 5a shows individual patient changes in HBsAg levels (CBL) in HBV patients treated with HBVS-219, 1.5 mg/kg per round from cohort C1 (averaged as light grey solid line in FIG. 4) against the cohort C1 placebo controls.

FIG. 5b shows individual patient changes of HBsAg levels (CBL) in HBV patients treated with HBVS-219, 3 mg/kg per round, from cohort C2 (averaged as medium grey solid line in FIG. 4) against the cohort C2 placebo controls. In FIG. 5b the results were adjusted for the average weight in the cohort tested, C2, (four 3 mg/kg per round patients and two placebo controls).

FIG. 5c shows individual patient changes of HBsAg levels (CBL) in HBV patients treated with HBVS-219, 6 mg/kg per round from cohort C3 (averaged as black solid line in FIG. 4) and the cohort C3 placebo controls. In FIG. 5c the results were adjusted for the average weight in C3.

FIG. 5d shows HBcrAg changes in Group C1 (NUC positive) treated patients.

FIG. 5e shows HBcrAg changes in Group C2 (NUC positive) treated patients.

FIG. 5f shows HBcrAg changes in Group C3 (NUC positive) treated patients.

FIG. 5g shows HBeAg changes in Group C1 (NUC positive) treated patients.

FIG. 5h shows HBeAg changes in Group C2 (NUC positive) treated patients.

FIG. 5i shows HBeAg changes in Group C3 (NUC positive) treated patients.

FIG. 5j shows HBV DNA changes in group C1 (NUC positive) treated patients.

FIG. 5k shows HBV DNA changes in group C2 (NUC positive) treated patients.

FIG. 5l shows HBV DNA changes in group C3 (NUC positive) treated patients.

FIG. 5m shows HBV RNA changes in group C1 (NUC positive) treated patients.

FIG. 5n shows HBV RNA changes in group C2 (NUC positive) treated patients.

FIG. 5o shows HBV RNA changes in group C3 (NUC positive) treated patients.

FIG. 6a shows mean changes in HBsAg from the baseline (CBL) of treatment in NUC-naïve HBV patients treated with a single dose monotherapy treatment of 3 mg/kg HBVS-219 or placebo for the entire cohort B1. Grey solid line is 3 mg/kg HBVS-219 treated average (n=6). Black dashed line is placebo treated average (n=3).

FIG. 6b shows individual changes in HBsAg levels (CBL) in NUC-naïve HBV patients treated with a single dose monotherapy treatment of 3 mg/kg HBVS-219 or placebo for the entire cohort B1. HBVS-219 treated patients are shown as solid lines (black and grey) and placebos are shown as dashed lines (black and grey). NUC-naïve patients in cohort B1 had no previous antiviral therapy for hepatitis B or previous HBV NUC or interferon-containing treatment.

FIG. 6c shows the individual patient changes in HBV DNA in cohort B1, monotherapy treatment of NUC-naïve patients. HBVS-219 treated patients are shown as solid lines (black and grey) and placebos are shown as dashed lines (black and grey).

FIG. 6d shows individual changes in HBcrAg levels (CBL) in cohort B1, monotherapy treatment of NUC-naïve patients. HBVS-219 treated patients are shown as solid lines (back and grey) and placebos are shown as dashed lines (black and grey).

FIG. 6e shows individual changes in HBeAg levels upon HBVS-219 administration in cohort B1, monotherapy treatment of NUC-naïve patients. Data are shown for patients from cohort B1 who were HBeAg positive at baseline. HBVS-219 treated patients are shown as solid lines (black and grey) and placebo is shown as dashed line (black).

FIG. 6f shows HBV RNA changes in group B (NUC naïve) treated patients.

FIG. 7 shows a reduction in levels of HBV DNA (solid dark grey line), a reduction in HBsAg (solid light grey line line) and a spike in ALT levels (solid black line) in a cohort B1 patient, MS76-467.

FIG. 8 shows the preservation of liver function as measurements of bilirubin (solid light grey line) and albumin (solid dark grey line, incompletely filled points) from a cohort B1 patient, MS76-467. ALT level changes are provided as the solid black line (in accordance with FIG. 7).

FIG. 9a shows a positive HBV Flare—ALT level increase (black solid line) with a corresponding reduction in HBsAg (solid light grey line) and no increase in HBV DNA (dark grey solid line) in a cohort B1 patient MS93-177.

FIG. 9b shows stability of liver function in cohort B1 patient MS93-177. Liver synthetic and excretory function levels provided as albumin shown as solid dark grey line with incompletely filled points, bilirubin shown as solid light grey line. ALT levels shown as black solid line (scale on right hand side).

FIG. 10 shows a time dependent overview of Injection Site-Related Adverse Events for group B1 and group C patients.

### DETAILED DESCRIPTION OF THE INVENTION

According to some aspects, the disclosure provides potent oligonucleotides for use in methods that are effective for reducing HBsAg expression in cells, particularly liver cells (e.g., hepatocytes) for the treatment of HBV infections and the induction of PHBV. In certain embodiments, HBsAg targeting oligonucleotides provided herein are designed for delivery to selected cells of target tissues (e.g., liver hepatocytes) to treat HBV infection in those tissues in an effective amount to achieve the desired therapeutic outcome.

According to the present invention, an effective amount of a pharmaceutical composition is administered to a subject in need thereof. The term “effective amount” means a sufficient amount to achieve the desired biological effect, which is here a curative or protective effect (in other words, an immunoprotecting effect), for example through induction of a positive seroconversion event and reduction of HBV viral load. It is understood that the effective dosage will be dependent upon the age, sex, health, and weight of the subject to be treated, the kind of concurrent treatment, if any, the frequency of treatment, and the nature of the expected effect. The ranges of effective doses provided herein are not intended to limit the invention and represent preferred dose ranges. However, the preferred dosage can be adapted to the subject, as it is understood and determinable by the one of skill in the art, without undue experimentation. See, e.g., Ebadi, PHARMACOLOGY, LITTLE, BROWN AND Co., BOSTON, MASS. EDS. (1985).

Accordingly, in related aspects, the disclosure provides methods of treating HBV infection that involve selectively reducing HBV surface antigen gene expression in cells (e.g., cells of the liver) to initiate a PHBV flare and improved prospects for recovery by the affected patient. This is particularly true for NUC-naïve patients where the current oligonucleotides of the invention are used as a monotherapy.

Further aspects of the disclosure, including a description of defined terms, are provided below.

### I. Definitions

Administering: As used herein, the terms “administering” or “administration” means to provide a substance (e.g., an oligonucleotide) to a subject in a manner that is pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Asialoglycoprotein Receptor (ASGPR): As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

Complementary: As used herein, the term “complementary” refers to a structural relationship between two nucleotides (e.g., on two opposing nucleic acids or on opposing regions of a single nucleic acid strand), or between two sequences of nucleotides, that permits the two nucleotides, or two sequences of nucleotides, to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other so as to form regions of complementarity, as described herein.

Deoxyribonucleotide: As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-Stranded Oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Flare: As used herein, the term “flare” or “ALT flare” is defined as a substantial alanine aminotransferase (ALT) elevation that is greater than 3-fold above the participant's baseline ALT value or greater than 3-fold above post-baseline nadir value (whichever value is lower), with an absolute ALT value that is at least 7×upper limit of normal (ULN), such as at least 10×ULN.

Hepatocyte: As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include but are not limited to: transthyretin (Ttr), glutamine synthetase (Glu1), hepatocyte nuclear factor 1a (Hnf1a), and hepatocyte nuclear factor 4a (Hnf4a). Markers for mature hepatocytes may include but are not limited to: cytochrome P450 (Cyp3a11), fumarylacetoacetate hydrolase (Fah), glucose 6-phosphate (G6p), albumin (Alb), and OC2-2F8. See, e.g., Huch et al., (2013), NATURE, 494(7436): 247-250, the contents of which relating to hepatocyte markers is incorporated herein by reference.

Hepatitis B Virus: As used herein, the term “Hepatitis B Virus” or “HBV” refers to a small DNA virus belonging to the Hepadnaviridae family and classified as the type species of the genus Orthohepadnavirus. HBV virus particles (virions) comprise an outer lipid envelope and an icosahedral nucleocapsid core composed of protein. The nucleocapsid generally encloses viral DNA and a DNA polymerase that has reverse transcriptase activity similar to retroviruses. The HBV outer envelope contains embedded proteins which are involved in viral binding of, and entry into, susceptible cells. HBV, which attacks the liver, has been classified according to at least ten genotypes (A-J) based on sequence. In general, there are four genes encoded by the genome, which genes are referred to as C, P, S, and X. The core protein is encoded by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S encodes surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. These may have a ratio of 1:1:4 (Heermann et al, 1984).

Hepatitis B Virus Proteins: Hepatitis B Virus (HBV) proteins can be organized into several categories and functions. Polymerases function as a reverse transcriptase (RT) to make viral DNA from pre-genomic RNA (pgRNA), and also as a DNA-dependent polymerase to make covalently closed circular DNA (cccDNA) from viral DNA. They are covalently attached to the 5′ end of the minus strand. Core proteins make the viral capsid and the secreted E antigen. Surface antigens are the hepatocyte internalization ligands, and also the primary component of aviral spherical and filamentous particles. Aviral particles are produced >1000-fold over Dane particles (infectious virions) and may act as immune decoys.

HBeAg Seroconversion: HBeAg seroconversion occurs when people infected with the HBeAg-positive form of the virus develop antibodies against the ‘e’ antigen. The seroconverted disease state is referred to as the ‘inactive HBV carrier state’ when HBeAg has been cleared, anti-HBe is present and HBV DNA is undetectable or less than 2000 IU/ml

Hepatitis B Virus Surface Antigen: As used herein, the term “hepatitis B virus surface antigen” or “HBsAg” refers to an S-domain protein encoded by gene S (e.g., ORF S) of an HBV genome. Hepatitis B virus particles carry viral nucleic acid in core particles enveloped by three proteins encoded by gene S, which are the large surface, middle surface, and major surface proteins. Among these proteins, the major surface protein is generally about 226 amino acids and contains just the S-domain. Presence of surface antigen signifies the presence of intact virus in the circulation.

Hepatitis B e Antigen (HBeAg): As used herein Hepatitis B e antigen (HBeAg) is an indicator of viral replication, although some variant forms of the virus do not express HBeAg (see ‘HBeAg-negative chronic hepatitis B’ below). Active infection can be described as HBeAg-positive or HBeAg-negative according to whether HBeAg is secreted.

Infection: As used herein, the term “infection” reefs to the pathogenic invasion and/or expansion of microorganisms, such as viruses, in a subject. An infection may be lysogenic, e.g., in which viral DNA lies dormant within a cell. Alternatively, an infection may be lytic, e.g., in which viruses actively proliferates and causing destruction of infected cells. An infection may or may not cause clinically apparent symptoms. An infection may remain localized, or it may spread, e.g., through a subject's blood or lymphatic system. An individual having, for example, an HBV infection, can be identified by detecting one or more of viral load, surface antigen (HBsAg), e-antigen (HBeAg), and various other assays for detecting HBV infection known in the art. Assays for detection of HBV infection can involve testing serum or blood samples for the presence of HBsAg and/or HBeAg, and optionally further screening for the presence of one or more viral antibodies (e.g., IgM and/or IgG) to compensate for any periods in which an HBV antigen may be at an undetectable level.

Liver Inflammation: As used herein, the term “liver inflammation” or “hepatitis” refers to a physical condition in which the liver becomes swollen, dysfunctional, and/or painful, especially as a result of injury or infection, as may be caused by exposure to a hepatotoxic agent. Symptoms may include jaundice (yellowing of the skin or eyes), fatigue, weakness, nausea, vomiting, appetite reduction, and weight loss. Liver inflammation, if left untreated, may progress to fibrosis, cirrhosis, liver failure, or liver cancer.

Liver Fibrosis: As used herein, the term “liver fibrosis” or “fibrosis of the liver” refers to an excessive accumulation in the liver of extracellular matrix proteins, which could include collagens (I, III, and IV), fibronectin, undulin, elastin, laminin, hyaluronan, and proteoglycans resulting from inflammation and liver cell death. Liver fibrosis, if left untreated, may progress to cirrhosis, liver failure, or liver cancer.

Loop: As used herein, the term “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

Nicked Tetraloop Structure: A “nicked tetraloop structure” is a structure of a RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

Nucleot(s)ide analogue: as used herein, also abbreviated to NUC herein, refers to nucleotide and nucleoside analogues. Nucleotide and nucleoside analogues are used as antiviral drugs (antiviral products), including for the treatment of HBV infection. Non limiting examples of nucleoside analogues which may be used for the treatment of HBV infection include Entecavir, Lamivudine, and Telbivudine. Non limiting examples of nucleotide analogues which may be used for the treatment of HBV infection include Tenofovir, such as Tenofovir disoproxil fumarate (TDF), Tenofovir alafenamide, and Adefovir dipivoxil.

NUC-naïve: A “NUC-naïve” patient is defined as a patient who has received no previous antiviral therapy for hepatitis B or hepatitis B virus (HBV) infection.

NUC-positive: A “NUC-positive” or “NUC suppressed” patient is defined as a patient who has previously received nucleot(s)ide analogue (NUC) treatment (for example entecavir or tenofovir) continuously for at least 12 weeks.

Oligonucleotide: As used herein, the term “oligonucleotide” refers to a short nucleic acid, e.g., of less than 100 nucleotides in length. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotides.

Pharmaceutically Acceptable Salt: The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphor sulfonate, citrate, cyclopentanepropionate, digluconate, dodecyl sulfate, ethane sulfonate, formate, fumarate, glucoheptonate, glycerophosphate, gluconate, hemi sulfate, heptanoate, hexanoate, hydroiodide, 2-hydroxy-ethane sulfonate, lactobionate, lactate, laurate, lauryl sulfate, malate, maleate, malonate, methane sulfonate, 2-naphthalene sulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluene sulfonate, undecanoate, valerate salts, and the like. Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Phosphate Analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, WO 2018/045317, and WO 2018/045317, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), NUCLEIC ACIDs RES., 43(6):2993-3011, the contents of each of which relating to phosphate analogs are incorporated herein by reference).

Reduced Expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to an HBsAg mRNA sequence) may result in a decrease in the amount of RNA transcript, protein and/or enzymatic activity (e.g., encoded by the S gene of an HBV genome) compared to a cell that is not treated with the double-stranded oligonucleotide. Similarly, “reducing expression” as used herein refers to an act that results in reduced expression of a gene (e.g., the S gene of an HBV genome).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions, e.g., in a phosphate buffer, in a cell, etc.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Sequentially: The administration of the two or more agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second and/or further agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

Seroconversion: HBeAg seroconversion occurs when people infected with the HBeAg-positive form of the virus develop antibodies against the ‘e’ antigen. The seroconverted disease state is referred to as the ‘inactive HBV carrier state’ when HBeAg has been cleared, anti-HBe is present and HBV DNA is undetectable or less than 2000 IU/ml.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” or “patient” refers to humans. The terms “individual” or “patient” may be used interchangeably with “subject.”

Synthetic: As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., NATURE 1990 Aug. 16; 346(6285):680-82; Heus and Pardi, SCIENCE 1991 Jul. 12; 253(5016):191-94). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) NUCL. ACIDS RES. 13: 3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., PROC NATL ACAD SC USA. 1990 November; 87(21):8467-71; Antao et al., NUCLEIC ACIDS RES. 1991 Nov. 11; 19(21):5901-05). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al., BIOCHEMISTRY, 41 (48), 14281-14292, 2002. SHINJI et al. NIPPON KAGAKKAI KOEN YOKOSHU VOL. 78th; NO. 2; PAGE. 731 (2000), which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., an existing HBV infection) or to prevent or decrease the likelihood of the occurrence of a condition (e.g., preventing liver fibrosis, hepatitis, liver cancer or other condition associated with an HBV infection). In some embodiments, treatment involves reducing the frequency or severity of at least one sign, symptom or contributing factor of a condition (e.g., HBV infection or related condition) experienced by a subject. During an HBV infection, a subject may exhibit symptoms such as yellowing of the skin and eyes (jaundice), dark urine, extreme fatigue, nausea, vomiting and abdominal pain. Accordingly, in some embodiments, a treatment provided herein may result in a reduction in the frequency or severity of one or more of such symptoms. However, HBV infection can develop into one or more liver conditions, such as cirrhosis, liver fibrosis, liver inflammation or liver cancer. Accordingly, in some embodiments, a treatment provided herein may result in a reduction in the frequency or severity of, or prevent or attenuate, one or more of such conditions.

Viral Load: The term “viral load” refers to the concentration of a virus, such as HBV, in the blood.

### II. Doses and Dosage Regimens

### Dose

The oligonucleotide according to the invention is administered in a defined dose. The term dose is used to refer to a unit of mass according to the patient's weight, expressed in mg/kg. The term dose is also used when referring to a fixed dose (or absolute dose amount), expressed in mg. The dose according to the invention may be a range and/or a single value.

### Initial Dose Range

The oligonucleotide for use according to the invention is administered at an initial dose from about 0.1 mg/kg to about 12 mg/kg. The initial dose may be from about 0.5 mg/kg to about 10 mg/kg. The initial dose may be from about 1.5 mg/kg to about 6 mg/kg. The oligonucleotide for use according to the invention may have an initial dose of about 1.5 mg/kg. The initial dose may be about 3 mg/kg. The initial dose may be about 6 mg/kg. The initial dose may be 0.5, 1, 1.5, 2, 2.5, 3, 3.5, 4, 4.5, 5, 5.5, 6, 7, 8, 9, 10, 11 or 12 mg/kg.

Expressed in a different manner, the oligonucleotide for use according to the invention is administered at an initial dose of from about 6 mg to about 800 mg. The initial dose may be from about 34 mg to about 667 mg. The initial dose may be from about 100 mg to about 400 mg. The oligonucleotide for use according to the invention, may have an initial dose is about 100 mg. The initial dose may be about 200 mg. The initial dose may be about 400 mg. The initial dose may be about 6, 7, 30, 34, 35, 50, 90, 100, 105, 150, 180, 200, 210, 236, 250, 300, 350, 400, 420, 450, 500, 550, 600, 650, 667, 700, 720, 750 or 800 mg.

### Initial and Subsequent Dose

In some embodiments, the invention relates to the administration of an initial dose and one or more subsequent doses.

The oligonucleotide for use according to the invention may comprise administering to the patient one or more subsequent doses of the oligonucleotide in an amount that is from about 0.1 mg/kg to about 12 mg/kg. The subsequent dose(s) may be from about 0.5 mg/kg to about 10 mg/kg. The subsequent dose(s) may be from about 1.5 mg/kg to about 6 mg/kg. The subsequent dose(s) may be about 1.5 mg/kg. The subsequent dose(s) may be about 3 mg/kg. The subsequent dose(s) may be about 6 mg/kg. The subsequent dose(s) may be 0.5, 1, 1.5, 2, 2.5, 3, 3.5, 4, 4.5, 5, 5.5, 6, 7, 8, 9, 10, 11 or 12 mg/kg.

The amount of each of the initial and subsequent doses may be the same or may be different and may be independently selected from the group consisting of: about 1.5 mg/kg, about 3 mg/kg and about 6 mg/kg.

The oligonucleotide for use according to the invention may further comprise administering to the patient one or more subsequent doses of the oligonucleotide in an amount that is from about 6 mg to about 800 mg. The subsequent dose(s) may be from about 34 mg to about 667 mg. The subsequent dose(s) may be from about 100 mg to about 400 mg.

The subsequent dose(s) may be about 100 mg. The subsequent dose(s) may be about 200 mg. The subsequent dose(s) may about 400 mg. The subsequent dose(s) may be 50, 100, 150, 200, 250, 300, 350, 400, 500, 600, 700 or 800 mg.

The amount of each of the initial and subsequent doses may be the same or may be different and may be independently selected from the group consisting of: about 100 mg, about 200 mg and about 400 mg.

There is provided an oligonucleotide for use according to the invention, wherein subsequent dose(s) may be about 6, 7, 30, 34, 35, 50, 90, 100, 105, 150, 180, 200, 210, 236, 250, 300, 350, 400, 420, 450, 500, 550, 600, 650, 667, 700, 720, 750 or 800 mg.

### Dose Timing

In some embodiments, the invention relates to an initial dose and one or more subsequent doses that are administered separated in time from each other.

The doses may be separated in time from each other by at least about four weeks. The doses may be separated in time from each other by at least about one month. The doses may be separated in time from each other by at least about two months. The doses may be separated in time from each other by at least about three months. The doses may be separated in time from each other by at least about six months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

### Dosage Regimen

In some embodiments, the invention relates to a dosage regimen.

The oligonucleotide for use according to the invention may be administered according to a dosage regimen which provides or achieves an effective treatment, cure or functional cure for hepatitis B or HBV infection.

The doses may be separated in time from each other by at least about four weeks, such as about four weeks, and be administered over a period of about 48 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about one month, such as about one month, and be administered over a period of about 48 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about two months, such as about two months, and be administered over a period of about 48 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about three months, such as about three months and be administered over a period of about 48 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about four weeks, such as about four weeks and be administered over a period of about 24 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about one month, such as about one month, and be administered over a period of about 24 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about two months, such as about two months, and be administered over a period of about 24 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about three months, such as about three months, and be administered over a period of about 24 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by about four weeks and be administered over a period of about three months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about one month, such as about one month, and be administered over a period of about three months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about four weeks, such as about four weeks, and be administered over a period of about 12 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time from each other by at least about one month, such as about one month, and be administered over a period of about 12 weeks. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time, each by a period of from about four weeks to about one month. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time, each by a period of from about four weeks to about two months, for example from about one month to about two months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The doses may be separated in time, each by a period of from about four weeks to about three months, for example from about one month to about three months, for example from about two months to about three months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

There is provided an oligonucleotide for use according to the invention, wherein the doses may be separated in time, each by a period of from about four weeks to about six months, for example from about one month to about six months, for example from about two months to about six months, for example from about three months to about six months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

The period of time between each of the doses may be independently selected from the group consisting of: about four weeks, about one month, about two months, about three months or about six months. Each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

There is provided an oligonucleotide for use according to the invention, wherein the period of time between each of the doses is as shown in any one of the treatment regimens A-O in Table 1 below. In the context of Table 1, each of the doses may be the same and may be selected from an amount of about 1.5 mg/kg, about 3 mg/kg or about 6 mg/kg. Each of the doses may be the same and may be selected from an amount of about 100 mg, about 200 mg or about 400 mg.

TABLE 1

| | | | Treatment holiday period (period in months after last |
|---|---|---|---|
| TR | NOD | TD | dose is administered according to TR, NOD and TD) |
| A | 1 | NA | 1, 2, 3, 4, 5, 6, 7, 8, 9, 10, 11, 12, 13, 14, 15, 16, 17 |
| B | 2 | 4 W | or 18 |
| C | 3 | 4 W | |
| D | 4 | 4 W | |
| E | 5 | 4 W | |
| F | 1 | 8 W | |
| G | 2 | 8 W | |
| H | 3 | 8 W | |
| I | 4 | 8 W | |
| J | 5 | 8 W | |
| K | 1 | NA | |
| L | 2 | V1 | |
| M | 3 | V2 | |
| N | 4 | V2 | |
| O | 5 | V2 | |

TR = treatment regimen,
NOD = Number of doses,
NA = not applicable,
TD = timing between doses,
V1-V2 = variable timing between doses,
V1 = 4 or 8 or 12 or 16 or 20 or 24 weeks,
V2 = 4 or 8 or 12 or 16 or 20 or 24 weeks between each dose (for example in Regimen M, first dose administered then 4 week period before second dose administered then 8 week period before third dose administered).

There is provided an oligonucleotide for use according to the invention, comprising administering to the patient at least one, at least two, at least three, at least four, at least five, at least six, at least seven, at least eight, at least nine or at least ten subsequent doses. In reality, any number of doses or subsequent doses may be administered, for example until an effective treatment, cure or subsequent cure is provided. Any of the dosage regimes provided herein may be repeated, for example until an effective treatment, cure, functional cure, endpoint or surrogate endpoint is achieved or provided.

There is provided an oligonucleotide for use according to the invention, wherein the method comprises a treatment holiday, preferably of about three to about six months. The treatment holiday may be the length disclosed in any one of regimen A-O in Table 1.

The period of time between the initial dose and each of between one and ten, preferably three, subsequent doses may be at least about four weeks, the method further comprising a treatment holiday of about three to about six months, after which administration of the oligonucleotide is recommenced.

The recommenced administration may comprise between one and ten, preferably three, subsequent doses, preferably wherein each recommenced subsequent dose is separated by a period of time of at least about four weeks.

The concept of “treatment holidays” may also be described by the skilled person in terms of “on-periods” and “off-periods”, wherein during an “on-period”, the patient is undergoing an active program of treatment, such as the four-weekly or monthly dosing regimens described herein. During an “off-period”, the patient takes a treatment holiday in which the patient is not undergoing active treatment. Such off-periods of treatment holidays may also be described as a treatment interruption. During a treatment holiday or interruption, a patient may be monitored for symptoms or biomarkers of HBV, in particular to determine the need to re-commence treatment. Suitable biomarkers are described herein.

### Monotherapy

In some embodiments the invention relates to a monotherapy.

There is provided an oligonucleotide for use according to the invention, wherein the initial dose may be a single dose or may be the only dose administered.

There is provided an oligonucleotide for use according to the invention, wherein the method may comprise or consist of or consist essentially of administering the oligonucleotide.

There is provided an oligonucleotide for use according to the invention, wherein the method may consist of or consist essentially of the administering the oligonucleotide, to the exclusion of other anti-viral or anti-HBV agents.

There is provided an oligonucleotide for use according to the invention, wherein the method may comprise, consist of or consist essentially of administering a single dose of the oligonucleotide.

There is provided an oligonucleotide for use according to the invention, wherein treatment, a cure or a functional cure of hepatitis B or HBV infection is provided by administering an initial dose, one dose or a single dose of the oligonucleotide.

There is provided an oligonucleotide for use according to the invention, wherein the treatment of hepatitis B or HBV infection is provided as a monotherapy.

There is provided oligonucleotide for use according to the invention, wherein the oligonucleotide is administered as a monotherapy.

### Combination Therapy

In some embodiments, the invention relates to combination therapies.

There is provided an oligonucleotide for use according to the invention, wherein the method further comprises administering an effective amount of at least one additional therapeutic agent. The additional therapeutic agent may be an antiviral agent. The antiviral agent may be an additional anti-HBV agent. The antiviral therapy may be one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; a TLR8 agonist; and a CpAM.

The interferon may be interferon alpha-2b, interferon alpha-2a, and interferon alphacon-1 (pegylated and unpegylated). Examples of IFN-α include, but not limited to, Pegasys© (Roche), PEG-Intron© (Merck& Co., Inc.) and Y-pegylated recombinant interferon alpha-2a (YPEG-IFNα-2a, Xiamen Amoytop Biotech Co., Ltd).

Anti-PDL1 antisense oligonucleotides are disclosed in WO2017157899 which is fully incorporated herein by reference. Preferably the anti PDL1 LNA antisense oligonucleotide is CMP ID NO: 768_2 disclosed in WO2017157899 or a pharmaceutically acceptable salt thereof. Preferably the PDL1 LNA antisense oligonucleotide comprises the sequence set forth in SEQ ID NO: 3. In a preferred embodiment, the anti PD-L1 antisense oligonucleotide has the formula GN2-C6ocoaoCCtatttaacatcAGAC (Compound I), wherein C6 represents an amino alkyl group with 6 carbons, capital letters represent beta-D-oxy LNA nucleosides, lowercase letters represent DNA nucleosides, all LNA C are 5-methyl cytosine, subscript o represents a phosphodiester nucleoside linkage, and unless otherwise indicated, all internucleoside linkages are phosphorothioate internucleoside linkages, and wherein GN2 represents the following trivalent GalNAc cluster:

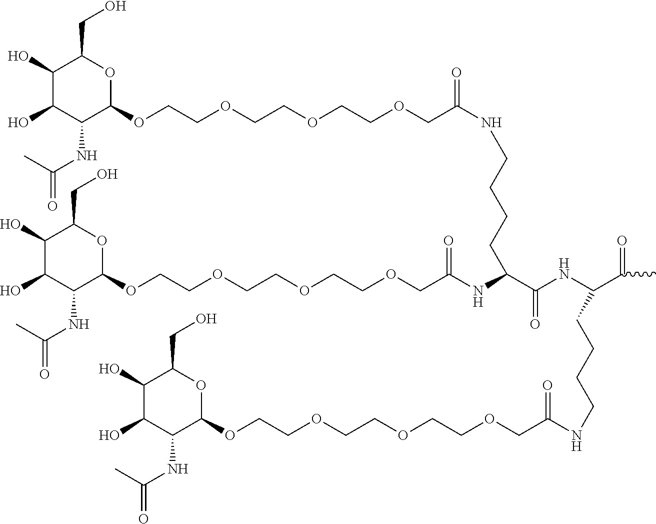

and further wherein the wavy line of the trivalent GalNAc cluster illustrates the site of conjugation of the trivalent GalNAc cluster to the C6 amino alkyl group; or a pharmaceutically acceptable salt thereof.

CpAM is disclosed in WO2015132276 which is fully incorporated herein by reference. Preferably CpAM is Compound II:

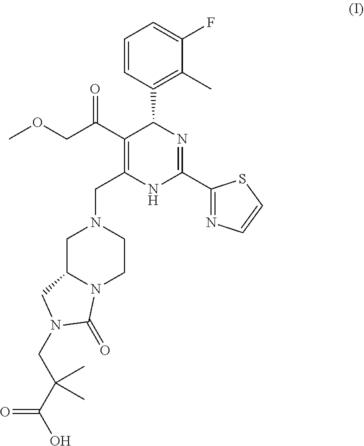 [SMILES: COCC(=O)C1=C(CN2CCN3C(=O)N(CC(C)(C)C(=O)O)C[C@@H]3C2)NC(c2nccs2)=N[C@H]1c1cccc(F)c1C]

or a pharmaceutically acceptable salt, enantiomer or diastereomer thereof.

The term “CpAM” denotes specifically Class I compounds of HBV core protein allosteric modulators that induce aberrant capsids subsequently degraded, including, but not limited to, GLS4 (Sunshine Pharma), QL-007 (Qilu), KL060332 (Sichuan Kelun Pharmaceutical) and Compound (II) which was disclosed in WO2015132276.

TLR7 agonists are disclosed in any one of JP2020100637, WO2018127526 and US20190169222, which are each fully incorporated herein by reference. Preferably, the TLR7 agonist is Compound III:

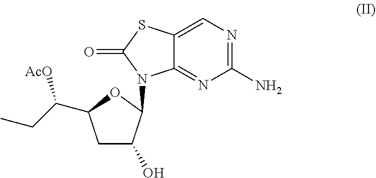 [SMILES: CC[C@H](OC(C)=O)[C@@H]1C[C@@H](O)[C@H](n2c(=O)sc3cnc(N)nc32)O1]

or a pharmaceutically acceptable salt, enantiomer or diastereomer thereof;

The antiviral therapy may be one or more of, for example all three of: Compound I; Compound II or a pharmaceutically acceptable salt, enantiomer or diastereomer thereof; and Compound III or a pharmaceutically acceptable salt, enantiomer or diastereomer thereof.

The additional therapeutic agent may be an antiviral agent. The antiviral agent may be an additional anti-HBV agent. The antiviral agent may be selected from interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; and an HBV antibody therapy (monoclonal or polyclonal). The antiviral agent may be a nucleot(s)ide analogue (NUC). The antiviral agent may be entecavir or pro-drug thereof or active thereof, tenofovir or pro-drug thereof or active thereof.

In one embodiment, the HBV antibody therapy is an antibody that binds to hepatitis B surface antigen (anti-HBsAg). The combination of the oligonucleotide and the anti-HBsAg antibody may lead to seroclearance of HBsAg in the patient. The anti-HbsAg antibody may be monoclonal. The anti-HBsAg antibody may be human.

In one embodiment the anti-HBsAg antibody comprises a heavy chain variable domain (VH) comprising (a) CDR-H1 comprising the amino acid sequence of SEQ ID NO:4, (b) CDR-H2 comprising the amino acid sequence of SEQ ID NO:5, and (c) CDR-H3 comprising the amino acid sequence of SEQ ID NO:6, and a light chain variable domain (VL) comprising (d) CDR-L1 comprising the amino acid sequence of SEQ ID NO:7, (e) CDR-L2 comprising the amino acid sequence of SEQ ID NO:8, and (f) CDR-L3 comprising the amino acid sequence of SEQ ID NO:9. In another embodiment, the antibody comprises a sequence selected from the group consisting of (a) a VH sequence having at least 95% sequence identity to the amino acid sequence of SEQ ID NO:19; (b) a VL sequence having at least 95% sequence identity to the amino acid sequence of SEQ ID NO:18; and (c) a VH sequence as defined in (a) and a VL sequence as defined in (b). In another embodiment, the antibody comprises a VH sequence of SEQ ID NO: 19 and a VL sequence of SEQ ID NO: 18. In another embodiment, the antibody comprises a heavy chain of SEQ ID NO:21 or 76 and a light chain of SEQ ID NO:20.

In one embodiment, the anti-HBsAg antibody comprises a heavy chain variable domain (VH) comprising (a) CDR-H1 comprising the amino acid sequence of SEQ ID NO:22, (b) CDR-H2 comprising the amino acid sequence of SEQ ID NO:23, and (c) CDR-H3 comprising the amino acid sequence of SEQ ID NO:24, and a light chain variable domain (VL) comprising (d) CDR-L1 comprising the amino acid sequence of SEQ ID NO:25, (e) CDR-L2 comprising the amino acid sequence of SEQ ID NO:26, and (f) CDR-L3 comprising the amino acid sequence of SEQ ID NO:27. In another embodiment, the antibody comprises a sequence selected from the group consisting of (a) a VH sequence having at least 95% sequence identity to the amino acid sequence of SEQ ID NO:37; (b) a VL sequence having at least 95% sequence identity to the amino acid sequence of SEQ ID NO:36; and (c) a VH sequence as defined in (a) and a VL sequence as defined in (b). In another embodiment, the antibody comprises a VH sequence of SEQ ID NO: 37 and a VL sequence of SEQ ID NO: 36. In another embodiment, the antibody comprises a heavy chain of SEQ ID NO:39 or 77 and a light chain of SEQ ID NO:38.

In one embodiment, the anti-HBsAg antibody comprises a heavy chain variable domain (VH) comprising (a) CDR-H1 comprising the amino acid sequence of SEQ ID NO:40, (b) CDR-H2 comprising the amino acid sequence of SEQ ID NO:41, and (c) CDR-H3 comprising the amino acid sequence of SEQ ID NO:42, and a light chain variable domain (VL) comprising (d) CDR-L1 comprising the amino acid sequence of SEQ ID NO:43, (e) CDR-L2 comprising the amino acid sequence of SEQ ID NO:44, and (f) CDR-L3 comprising the amino acid sequence of SEQ ID NO:45. In another embodiment, the antibody comprises a sequence selected from the group consisting of (a) a VH sequence having at least 95% sequence identity to the amino acid sequence of SEQ ID NO:55; (b) a VL sequence having at least 95% sequence identity to the amino acid sequence of SEQ ID NO:54; and (c) a VH sequence as defined in (a) and a VL sequence as defined in (b). In another embodiment, the antibody comprises a VH sequence of SEQ ID NO: 55 and a VL sequence of SEQ ID NO: 54. In another embodiment, the antibody comprises a heavy chain of SEQ ID NO:57 or 78 and a light chain of SEQ ID NO:56.

In one embodiment, the anti-HBsAg antibody comprises a heavy chain variable domain (VH) comprising (a) CDR-H1 comprising the amino acid sequence of SEQ ID NO:58, (b) CDR-H2 comprising the amino acid sequence of SEQ ID NO:59, and (c) CDR-H3 comprising the amino acid sequence of SEQ ID NO:60, and a light chain variable domain (VL) comprising (d) CDR-L1 comprising the amino acid sequence of SEQ ID NO:61, (e) CDR-L2 comprising the amino acid sequence of SEQ ID NO:62, and (f) CDR-L3 comprising the amino acid sequence of SEQ ID NO:63. In another embodiment, the antibody comprises a sequence selected from the group consisting of (a) a VH sequence having at least 95% sequence identity to the amino acid sequence of SEQ ID NO:73; (b) a VL sequence having at least 95% sequence identity to the amino acid sequence of SEQ ID NO:72; and (c) a VH sequence as defined in (a) and a VL sequence as defined in (b). In another embodiment, the antibody comprises a VH sequence of SEQ ID NO: 73 and a VL sequence of SEQ ID NO:72. In another embodiment, the antibody comprises a heavy chain of SEQ ID NO:75 or 79 and a light chain of SEQ ID NO:74.

In one embodiment, the antibody comprises a light chain as set out herein and a heavy chain set out herein, modified by substitutions selected from the group consisting of:
i) M252Y, S254T and T256E;
ii) M428L, N434A and Y436T;
iii) N434A; and
iv) T307H and N434H.
   The CDRs, framework regions (FW), VH, VL, heavy chains and light chains of certain antibodies according to the present invention are set out in Table 2 below:

TABLE 2
Antibody Sequences

| SEQ | | |
|---|---|---|
| ID | | |
| NO: | Description | Sequence |
| 4 | Bc1.187 CDR-H1 | NYGMQ |
| 5 | Bc1.187 CDR-H2 | IIWADGTKQYYGDSVKG |
| 6 | Bc1.187 CDR-H3 | DGLYASAPNDV |
| 7 | Bc1.187 CDR-L1 | RASQRISTYLN |
| 8 | Bc1.187-CDR-L2 | GASSLOS |
| 9 | Bc1.187 CDR-L3 | QQTYTLPPN |
| 10 | Bc1.187 H-FW1 | QVQLVESGGGVVQPGRSLRLSCEASGFTFS |
| 11 | Bc1.187 H-FW2 | WVRQAPGKGLEWVA |
| 12 | Bc1.187 H-FW3 | FTISRDNFKNTLYLQMNSLRGEDTAMYFCAR |
| 13 | Bc1.187 H-FW4 | WGQGTLVTVSS |
| 14 | Bc1.187 L-FW1 | DIQMTQSPSSLSAYVGDRVTITC |
| 15 | Bc1.187 L-FW2 | WYHQRPGKSPSLLIY |
| 16 | Bc1.187 L-FW3 | GVPSRFSASASGTDFTLTISSLRPEDLGTYYC |
| 17 | Bc1.187 L-FW4 | SGGGTKVEIK |
| 18 | Bc1.187 VL | DIQMTQSPSSLSAYVGDRVTITCRASQRISTYLNW |
| | | YHQRPGKSPSLLIYGASSLQSGVPSRFSASASGTD |
| | | FTLTISSLRPEDLGTYYCQQTYTLPPNSGGGTKVEI |
| | | K |
| 19 | Bc1.187 VH | QVQLVESGGGVVQPGRSLRLSCEASGFTFSNYGM |
| | | QWVRQAPGKGLEWVAIIWADGTKQYYGDSVKG |
| | | RFTISRDNFKNTLYLQMNSLRGEDTAMYFCARDG |
| | | LYASAPNDVWGQGTLVTVSS |
| 20 | Bc1.187 | DIQMTQSPSSLSAYVGDRVTITCRASQRISTYLNW |
| | full length | YHQRPGKSPSLLIYGASSLQSGVPSRFSASASGTD |
| | light chain | FTLTISSLRPEDLGTYYCQQTYTLPPNSGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 21 | Bc1.187 | QVQLVESGGGVVQPGRSLRLSCEASGFTFSNYGM |
| | full length | QWVRQAPGKGLEWVAIIWADGTKQYYGDSVKG |
| | heavy chain | RFTISRDNFKNTLYLQMNSLRGEDTAMYFCARDG |
| | | LYASAPNDVWGQGTLVTVSSASTKGPSVFPLAPS |
| | | SKSTSGGTAALGCLVKDYFPEPVTVSWNSGALTS |
| | | GVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTI |
| | | SKAKQPREPQVYTLPPSRDELTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPGK |
| 22 | Bc3.106 CDR-H1 | SYAMS |
| 23 | Bc3.106 CDR-H2 | AFSGTGGSTYYADSVKG |
| 24 | Bc3.106 CDR-H3 | DPGHTSNWRDNYQYYQMDV |
| 25 | Bc3.106 CDR-L1 | RASQGIRNDLG |
| 26 | Bc3.106-CDR-L2 | AASSLOS |
| 27 | Bc3.106 CDR-L3 | LOHNSYPRT |
| 28 | Bc3.106 H-FW1 | EVQLLESGGGLVQPGGSLRLSCTASGFTFG |
| 29 | Bc3.106 H-FW2 | WVRQAPGKGLKWVS |
| 30 | Bc3.106 H-FW3 | RFTISRDNSKNTLYLQMNNLRAEDTAVYFCAK |
| 31 | Bc3.106 H-FW4 | WGQGTTVTVSS |
| 32 | Bc3.106 L-FW1 | DIQMTQSPSSLSASVGDRVTITC |
| 33 | Bc3.106 L-FW2 | WYQQKPGKAPKRLIY |
| 34 | Bc3.106 L-FW3 | GVPSRFSGSGSGTEFTLTISSLOPEDFATYYC |
| 35 | Bc3.106 L-FW4 | FGQGTKVEIK |
| 36 | Bc3.106 VL | DIQMTQSPSSLSASVGDRVTITCRASQGIRNDLGW |
| | | YQQKPGKAPKRLIYAASSLQSGVPSRFSGSGSGTE |
| | | FTLTISSLQPEDFATYYCLQHNSYPRTFGQGTKVEI |
| | | K |
| 37 | Bc3.106 VH | EVQLLESGGGLVQPGGSLRLSCTASGFTFGSYAM |
| | | SWVRQAPGKGLKWVSAFSGTGGSTYYADSVKGR |
| | | FTISRDNSKNTLYLQMNNLRAEDTAVYFCAKDPG |
| | | HTSNWRDNYQYYQMDVWGQGTTVTVSS |
| 38 | Bc3.106 | DIQMTQSPSSLSASVGDRVTITCRASQGIRNDLGW |
| | full length | YQQKPGKAPKRLIYAASSLQSGVPSRFSGSGSGTE |
| | light chain | FTLTISSLOPEDFATYYCLQHNSYPRTFGQGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 39 | Bc3.106 | EVQLLESGGGLVQPGGSLRLSCTASGFTFGSYAM |
| | full length | SWVRQAPGKGLKWVSAFSGTGGSTYYADSVKGR |
| | heavy chain | FTISRDNSKNTLYLQMNNLRAEDTAVYFCAKDPG |
| | | HTSNWRDNYQYYQMDVWGQGTTVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPELLGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | PAPIEKTISKAKQPREPQVYTLPPSRDELTKNQVSL |
| | | TCLVKGFYPSDIAVEWESNGQPENNYKTTPPVLD |
| | | SDGSFFLYSKLTVDKSRWQQGNVFSCSVMHEAL |
| | | HNHYTQKSLSLSPGK |
| 40 | Bv4.115 CDR-H1 | NYHIH |
| 41 | Bv4.115 CDR-H2 | IINPRRLSTAYAPKFQG |
| 42 | Bv4.115 CDR-H3 | DAGDDTSGPFDS |
| 43 | Bv4.115 CDR-L1 | RASQSINTWLA |
| 44 | Bv4.115-CDR-L2 | KASSLES |
| 45 | Bv4.115 CDR-L3 | QQYNTFS |
| 46 | Bv4.115 H-FW1 | QVQLVQSGAEVKKPGSSVKVSCRSSGYRFT |
| 47 | Bv4.115 H-FW2 | WVRQAPGQGLEWVG |
| 48 | Bv4.115 H-FW3 | RVTMTRDTSTSTVYMELSSLRSDDTAVYYCAR |
| 49 | Bv4.115 H-FW4 | WGQGTLVTVSS |
| 50 | Bv4.115 L-FW1 | DIQMTQSPSTLSASVGDRVTITC |
| 51 | Bv4.115 L-FW2 | WYQQKPGKAPKLLIS |
| 52 | Bv4.115 L-FW3 | GVPSRFSGSGSGTEFTLSISSLQPDDFATYYC |
| 53 | Bv4.115 L-FW4 | FGQGTKLEIK |
| 54 | Bv4.115 VL | DIQMTQSPSTLSASVGDRVTITCRASQSINTWLAW |
| | | YQQKPGKAPKLLISKASSLESGVPSRFSGSGSGTE |
| | | FTLSISSLQPDDFATYYCQQYNTFSFGQGTKLEIK |
| 55 | Bv4.115 VH | QVQLVQSGAEVKKPGSSVKVSCRSSGYRFTNYHI |
| | | HWVRQAPGQGLEWVGIINPRRLSTAYAPKFQGRV |
| | | TMTRDTSTSTVYMELSSLRSDDTAVYYCARDAG |
| | | DDTSGPFDSWGQGTLVTVSS |
| 56 | Bv4.115h | DIQMTQSPSTLSASVGDRVTITCRASQSINTWLAW |
| | full length | YQQKPGKAPKLLISKASSLESGVPSRFSGSGSGTE |
| | light chain | FTLSISSLQPDDFATYYCQQYNTFSFGQGTKLEIKR |
| | | TVAAPSVFIFPPSDEQLKSGTASVVCLLNNFYPRE |
| | | AKVQWKVDNALQSGNSQESVTEQDSKDSTYSLS |
| | | STLTLSKADYEKHKVYACEVTHQGLSSPVTKSFN |
| | | RGEC |
| 57 | Bv4.115 | QVQLVQSGAEVKKPGSSVKVSCRSSGYRFTNYHI |
| | full length | HWVRQAPGQGLEWVGIINPRRLSTAYAPKFQGRV |
| | heavy chain | TMTRDTSTSTVYMELSSLRSDDTAVYYCARDAG |
| | | DDTSGPFDSWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKQPREPQVYTLPPSRDELTKNQVSLTCLVKGF |
| | | YPSDIAVEWESNGQPENNYKTTPPVLDSDGSFFLY |
| | | SKLTVDKSRWQQGNVFSCSVMHEALHNHYTQKS |
| | | LSLSPGK |
| 58 | Bc8.159 CDR-H1 | TNNWWS |
| 59 | Bc8.159 CDR-H2 | EIHHIGSTNYNPSLKS |
| 60 | Bc8.159 CDR-H3 | GRLGITRDRYYFDS |
| 61 | Bc8.159 CDR-L1 | QASQDISNYLN |
| 62 | Bc8.159-CDR-L2 | DTSSLER |
| 63 | Bc8.159 CDR-L3 | QQYYNLPHT |
| 64 | Bc8.159 H-FW1 | QVQLQESGPGLVKPSGTLSLTCAVSGGTIR |
| 65 | Bc8.159 H-FW2 | WVROPPGKGLEWIG |
| 66 | Bc8.159 H-FW3 | QVTISVDKSKNQFSLNLSSVTAADTALYYCVR |
| 67 | Bc8.159 H-FW4 | WGRGTLVTVSS |
| 68 | Bc8.159 L-FW1 | DIQMTQSPSPLSVSVGDRVTITC |
| 69 | Bc8.159 L-FW2 | WYQQKPGQAPKLLIY |
| 70 | Bc8.159 L-FW3 | GVPSRFSGSGSGTDFTLTISSLOPEDIATYHC |
| 71 | Bc8.159 L-FW4 | FGQGTKLEIK |
| 72 | Bc8.159 VL | DIQMTQSPSPLSVSVGDRVTITCQASQDISNYLNW |
| | | YQQKPGQAPKLLIYDTSSLERGVPSRFSGSGSGTD |
| | | FTLTISSLQPEDIATYHCQQYYNLPHTFGQGTKLEI |
| | | K |
| 73 | Bc8.159 VH | QVQLQESGPGLVKPSGTLSLTCAVSGGTIRTNNW |
| | | WSWVRQPPGKGLEWIGEIHHIGSTNYNPSLKSQV |
| | | TISVDKSKNQFSLNLSSVTAADTALYYCVRGRLGI |
| | | TRDRYYFDSWGRGTLVTVSS |
| 74 | Bc8.159 | DIQMTQSPSPLSVSVGDRVTITCQASQDISNYLNW |
| | full length | YQQKPGQAPKLLIYDTSSLERGVPSRFSGSGSGTD |
| | light chain | FTLTISSLQPEDIATYHCQQYYNLPHTFGQGTKLEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 75 | Bc8.159 | QVQLQESGPGLVKPSGTLSLTCAVSGGTIRTNNW |
| | full length | WSWVRQPPGKGLEWIGEIHHIGSTNYNPSLKSQV |
| | heavy chain | TISVDKSKNQFSLNLSSVTAADTALYYCVRGRLGI |
| | | TRDRYYFDSWGRGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKQPREPQVYTLPPSRDELTKNQVSLTCLVKGF |
| | | YPSDIAVEWESNGQPENNYKTTPPVLDSDGSFFLY |
| | | SKLTVDKSRWQQGNVFSCSVMHEALHNHYTQKS |
| | | LSLSPGK |
| 76 | Bc1.187 | QVQLVESGGGVVQPGRSLRLSCEASGFTFSNYG |
| | full length | MQWVRQAPGKGLEWVAIIWADGTKQYYGDSVKG |
| | heavy chain | RFTISRDNFKNTLYLQMNSLRGEDTAMYFCARDG |
| | (containing | LYASAPNDVWGQGTLVTVSSASTKGPSVFPLAPS |
| | modifications) | SKSTSGGTAALGCLVKDYFPEPVTVSWNSGALTS |
| | | GVHTFPAVLQSSGLYSLSSVVTVPSSSLGTQTYIC |
| | | NVNHKPSNTKVDKRVEPKSCDKTHTCPPCPAPEL |
| | | LGGPSVFLFPPKPKDTLMISRTPEVTCVVVDVSH |
| | | EDPEVKFNWYVDGVEVHNAKTKPREEQYNSTYRV |
| | | VSVLTVLHQDWLNGKEYKCKVSNKALPAPIEKTI |
| | | SKAKGQPREPQVYTLPPSREEMTKNQVSLTCLVK |
| | | GFYPSDIAVEWESNGQPENNYKTTPPVLDSDGSF |
| | | FLYSKLTVDKSRWQQGNVFSCSVMHEALHNHYTQ |
| | | KSLSLSPGK |
| 77 | Bc3.106 | EVQLLESGGGLVQPGGSLRLSCTASGFTFGSYAM |
| | full length | SWVRQAPGKGLKWVSAFSGTGGSTYYADSVKGR |
| | heavy chain | FTISRDNSKNTLYLQMNNLRAEDTAVYFCAKDPG |
| | (containing | HTSNWRDNYQYYQMDVWGQGTTVTVSSASTKG |
| | modifications) | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKRVEPKSCDKTHTC |
| | | PPCPAPELLGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | PAPIEKTISKAKGQPREPQVYTLPPSREEMTKNQV |
| | | SLTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| 78 | Bv4.115 | QVQLVQSGAEVKKPGSSVKVSCRSSGYRFTNYHI |
| | full length | HWVRQAPGQGLEWVGIINPRRLSTAYAPKFQGRV |
| | heavy chain | TMTRDTSTSTVYMELSSLRSDDTAVYYCARDAG |
| | (containing | DDTSGPFDSWGQGTLVTVSSASTKGPSVFPLAPSS |
| | modifications) | KSTSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPGK |
| 79 | Bc8.159 | QVQLQESGPGLVKPSGTLSLTCAVSGGTIRTNNW |
| | full length | WSWVRQPPGKGLEWIGEIHHIGSTNYNPSLKSQV |
| | heavy chain | TISVDKSKNQFSLNLSSVTAADTALYYCVRGRLGI |
| | (containing | TRDRYYFDSWGRGTLVTVSSASTKGPSVFPLAPSS |
| | modifications) | KSTSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPGK |

The additional therapeutic agent may selected from an antiviral agent, a reverse transcriptase inhibitor, an immune stimulator, a therapeutic vaccine, a viral entry inhibitor, an oligonucleotide that inhibits the secretion or release of HBsAg, a capsid inhibitor, a cccDNA inhibitor, and a combination of any of the foregoing. The additional therapeutic agent may be a reverse transcriptase inhibitor and an immune stimulator. The reverse transcriptase inhibitor may be selected from the group consisting of Tenofovir disoproxil fumarate (TDF), Tenofovir alafenamide, Lamivudine, Adefovir dipivoxil, Entecavir (ETV), Telbivudine, and AGX-1009. The immune stimulator may be selected from the group consisting of pegylated interferon alpha2a, Interferon alpha2b, a recombinant human interleukin-7, a Toll-like receptor 7 (TLR7) agonist, and a Toll-like receptor 8 (TLR8) agonist.

The additional therapeutic agent may be administered according to the same or different dosing regimen as the oligonucleotide. The additional therapeutic agent may be administered together in a single formulation, or separately in different formulations. The oligonucleotide and the additional therapeutic agent may be administered concomitantly. The oligonucleotide and the additional therapeutic agent may be administered sequentially.

In one embodiment, the additional therapeutic agent is a therapeutic vaccine, wherein the oligonucleotide and the therapeutic vaccine are administered sequentially, preferably wherein between 1-3 doses of the therapeutic vaccine are administered following administration of the oligonucleotide.

The period of time between the administration of the oligonucleotide and the additional therapeutic agent may be about four weeks, about one month, about two months, about 12 weeks, about three months, about 24 weeks or about 6 months, preferably about 12 weeks.

The method may comprise a monotherapy lead-in phase, wherein one or more doses of the oligonucleotide may be administered prior to the first dose of any additional therapeutic agent. The method may comprise a monotherapy lead-in phase, wherein one or more doses of the oligonucleotide are administered prior to a second dose of the additional therapeutic agent.

When administered on the same day, the oligonucleotide and the additional therapeutic agent are administered simultaneously at least once. When administered on the same day, the oligonucleotide and the additional therapeutic agent are administered sequentially at least once.

The oligonucleotide and the additional therapeutic agent may be administered together in a single combination formulation. The oligonucleotide and the additional therapeutic agent may be administered separately in different formulations.

There is provided an oligonucleotide for use according to the invention, wherein the patient has not previously have been treated with an antiviral therapy, wherein the patient is administered an initial dose of the oligonucleotide of about 3 mg/kg followed by three subsequent doses of the oligonucleotide of about 3 mg/kg, wherein the doses are separated in time from each other by a period of about four weeks.

There is provided an oligonucleotide for use according to the invention, wherein the patient has not previously been treated with an antiviral therapy, wherein the patient is administered an initial dose of the oligonucleotide of about 6 mg/kg followed by three subsequent doses of the oligonucleotide of about 6 mg/kg, wherein the doses are separated in time from each other by a period of about four weeks.

There is provided an oligonucleotide for use according to according to the invention, wherein the patient has not previously been treated with an antiviral therapy, wherein the patient is administered an initial dose of the oligonucleotide of about 100 mg followed by three subsequent doses of the oligonucleotide of about 100 mg, wherein the doses are separated in time from each other by a period of about four weeks.

There is provided an oligonucleotide for use according to the invention, wherein the patient has not previously been treated with an antiviral therapy, wherein the patient is administered an initial dose of the oligonucleotide of about 200 mg followed by three subsequent doses of the oligonucleotide of about 200 mg, wherein the doses are separated in time from each other by a period of about four weeks.

There is provided an oligonucleotide for use according to the invention, wherein the patient has not previously been treated with an antiviral therapy, wherein the patient is administered an initial dose of the oligonucleotide of about 400 mg followed by three subsequent doses of the oligonucleotide of about 400 mg, wherein the doses are separated in time from each other by a period of about four weeks.

### Disease and Patient Groups

The invention relates to oligonucleotides for use in the treatment of human patients with Hepatitis B or HBV infection. The disease may be acute or chronic. Embodiments of the invention relate to the treatment of patient subgroups.

There is provided an oligonucleotide for use according to the invention, wherein the patient has chronic hepatitis B or has a chronic HBV infection. The chronic hepatitis B patient may be treatment naïve, nucleot(s)ide analogue (NUC) suppressed, immune active, cirrhotic, immuno-tolerant, an inactive carrier or HBV delta co-infection.

The patient may be treatment naïve. The patient may not have previously been treated with an antiviral therapy. The antiviral therapy may be an anti-HBV therapy. The patient may not have previously been treated with an antiviral therapy for a period of at least about six months. The antiviral therapy may be a nucleot(s)ide analogue (NUC), or an interferon-containing agent. The antiviral therapy may be one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; and a CpAM. Preferably, the antiviral therapy is entecavir or pro-drug thereof or active thereof, tenofovir or pro-drug thereof or active thereof.

The patient may be immune active. The term immune active is well known in the art as a disease phase when the human patient immune system recognizes HBV as foreign and tries to eradicate HBV, but the cytotoxic response is considered to be weak. The immune active phase may be confirmed by elevated or fluctuating levels of ALT, HBV DNA levels more than 2000 IU/mL (commonly more than 20,000 IU/ml) and active liver inflammation. The immune active human patient may be HBeAg positive or HBeAg negative. The immune active patient may be a chronic HBV patient.

The patient may be cirrhotic. The term cirrhotic is well known in the art. A cirrhotic patient has long term damage such that the liver that does not function properly. Long term refers to development over a period of months or more. The human cirrhotic patient may be HBeAg positive or HBeAg negative. The cirrhotic patient may be a chronic HBV patient.

The patient may be immuno-tolerant. The patient may be immuno-tolerant. The human immuno-tolerant patient is known in the art to be HBeAg positive. The human immuno-tolerant patient may further be confirmed by HBV DNA levels at or above 20,000 IU/ml and no significant immune response to the virus. The human immune-tolerant patient may further have persistently normal ALT levels. The immune-tolerant patient may be a chronic HBV patient.

The patient may be HBV delta co-infection. The term HBV delta co-infection is known in the art to define a patient that is co-infected with HBV and hepatitis D virus (HDV).

The patient may be nucleot(s)ide analogue (NUC) suppressed (also referred to herein as NUC-positive). The NUC-positive patient may be HBeAg positive or HBeAg negative.

The patient may be an inactive carrier. The inactive carrier patient may be HBeAg negative. The inactive carrier state may further be confirmed by the presence of anti-HBe, undetectable or low levels of HBV DNA in PCR-based assays, repeatedly normal ALT levels, and minimal or no necroinflammation, slight fibrosis or even normal histology on biopsy.

There is provided an oligonucleotide for use according to the invention, wherein the human hepatitis B virus-related condition is selected from any of: jaundice, liver cancer, liver inflammation, liver fibrosis, liver cirrhosis, liver failure, diffuse hepatocellular inflammatory disease, hemophagocytic syndrome or serum hepatitis.

### Subcutaneous Administration

The oligonucleotide for use according to the invention may be administered to the patient via the subcutaneous route. Administration via the subcutaneous route may be in the thigh or abdomen. Preferably the administration is by subcutaneous injection.

### Specific Embodiments

The invention provides a number of specific, non-limiting, embodiments.

In one embodiment, there is provided an oligonucleotide for use according to the invention, wherein the patient has not previously been treated with an antiviral therapy, wherein the patient is administered an initial dose of the oligonucleotide of about 1.5 mg/kg followed by three subsequent doses of the oligonucleotide of about 1.5 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the patient is administered an initial dose of the oligonucleotide of about 3 mg/kg followed by three subsequent doses of the oligonucleotide of about 3 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the patient is administered an initial dose of the oligonucleotide of about 6 mg/kg followed by three subsequent doses of the oligonucleotide of about 6 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the method consists of the administration of one dose of the oligonucleotide in an amount of about 1.5 mg/kg.

In one embodiment, there is provided oligonucleotide for use according to the invention wherein the patient has not previously been treated with an antiviral therapy, wherein the method consists of the administration of one dose of the oligonucleotide in an amount of about 3 mg/kg.

In one embodiment, there is provided an oligonucleotide for use according to the invention, wherein the patient has not previously been treated with an antiviral therapy, wherein the method consists of the administration of one dose of the oligonucleotide in an amount of about 6 mg/kg.

In one embodiment, there is provided an oligonucleotide for use according to the invention, wherein the patient has not previously been treated with an antiviral therapy, wherein the method comprises the administration of a single dose of the oligonucleotide in an amount of about 1.5 mg/kg. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient has not previously been treated with an antiviral therapy, wherein the method comprises the administration of a single dose of the oligonucleotide in an amount of about 3 mg/kg. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient has not previously been treated with an antiviral therapy, wherein the method comprises the administration of a single dose of the oligonucleotide in an amount of about 6 mg/kg. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient has not previously been treated with an antiviral therapy, wherein the patient is administered an initial dose of the oligonucleotide of about 90 mg or about 100 mg followed by three subsequent doses of the oligonucleotide of about 90 mg or about 100 mg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the patient is administered an initial dose of the oligonucleotide of about 200 mg or about 210 mg followed by three subsequent doses of the oligonucleotide of about 200 mg or about 210 mg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the patient is administered an initial dose of the oligonucleotide of about 360 mg or about 400 mg followed by three subsequent doses of the oligonucleotide of about 360 mg or about 400 mg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient has not previously been treated with an antiviral therapy, wherein the method consists of the administration of one dose of the oligonucleotide in an amount of about 90 mg or about 100 mg.

In one embodiment, there is provided an oligonucleotide for according to the invention, wherein the patient has not previously been treated with an antiviral therapy, wherein the method consists of the administration of one dose of the oligonucleotide in an amount of about 200 mg or about 210 mg.

In one embodiment, there is provided an oligonucleotide for use according to the invention, wherein the patient has not previously been treated with an antiviral therapy, wherein the method consists of the administration of one dose of the oligonucleotide in an amount of about 360 mg or about 400 mg.

In one embodiment, there is provided an oligonucleotide for use according to the invention, wherein the patient has not previously been treated with an antiviral therapy, wherein the method comprises the administration of a single dose of the oligonucleotide in an amount of about 90 mg or about 100 mg. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient has not previously been treated with an antiviral therapy, wherein the method comprises the administration of a single dose of the oligonucleotide in an amount of about 200 mg or about 210 mg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient has not previously been treated with an antiviral therapy, wherein the method comprises the administration of a single dose of the oligonucleotide in an amount of about 360 mg or about 400 mg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the patient is administered an initial dose of the oligonucleotide of about 1.5 mg/kg followed by three subsequent doses of the oligonucleotide of about 1.5 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient is NUC-suppressed, wherein the patient is administered an initial dose of the oligonucleotide of about 3 mg/kg followed by three subsequent doses of the oligonucleotide of about 3 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient is NUC-suppressed, wherein the patient is administered an initial dose of the oligonucleotide of about 6 mg/kg followed by three subsequent doses of the oligonucleotide of about 6 mg/kg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide (e.g. the fourth dose of the oligonucleotide) and administration of the antiviral agent is about 12 weeks.

In one embodiment, there is provided an oligonucleotide for use according to the invention, wherein the patient is NUC-suppressed, wherein the method consists of the administration of one dose of the oligonucleotide in an amount of about 1.5 mg/kg.

In one embodiment, there is provided oligonucleotide for use according to the invention wherein the patient is NUC-suppressed, wherein the method consists of the administration of one dose of the oligonucleotide in an amount of about 3 mg/kg.

In one embodiment, there is provided an oligonucleotide for use according to the invention, wherein the patient is NUC-suppressed, wherein the method consists of the administration of one dose of the oligonucleotide in an amount of about 6 mg/kg.

In one embodiment, there is provided an oligonucleotide for use according to the invention, wherein the patient is NUC-suppressed, wherein the method comprises the administration of a single dose of the oligonucleotide in an amount of about 1.5 mg/kg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the method comprises the administration of a single dose of the oligonucleotide in an amount of about 3 mg/kg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the method comprises the administration of a single dose of the oligonucleotide in an amount of about 6 mg/kg. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the patient is administered an initial dose of the oligonucleotide of about 90 mg or about 100 mg followed by three subsequent doses of the oligonucleotide of about 90 mg or about 100 mg, wherein the doses are separated in time from each other by a period of about four weeks. The method may further comprise the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the patient is administered an initial dose of the oligonucleotide of about 200 mg or about 210 mg followed by three subsequent doses of the oligonucleotide of about 200 mg or about 210 mg, wherein the doses are separated in time from each other by a period of about four weeks. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the patient is administered an initial dose of the oligonucleotide of about 360 mg or about 400 mg followed by three subsequent doses of the oligonucleotide of about 360 mg or about 400 mg, wherein the doses are separated in time from each other by a period of about four weeks. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the method consists of the administration of one dose of the oligonucleotide in an amount of about 90 mg or about 100 mg.

In one embodiment, there is provided an oligonucleotide for according to the invention, wherein the patient is NUC-suppressed, wherein the method consists of the administration of one dose of the oligonucleotide in an amount of about 200 mg or about 210 mg.

In one embodiment, there is provided an oligonucleotide for use according to the invention, wherein the patient is NUC-suppressed, wherein the method consists of the administration of one dose of the oligonucleotide in an amount of about 360 mg or about 400 mg.

In one embodiment, there is provided an oligonucleotide for use according to the invention, wherein the patient is NUC-suppressed, wherein the method comprises the administration of a single dose of the oligonucleotide in an amount of about 90 mg or about 100 mg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the method comprises the administration of a single dose of the oligonucleotide in an amount of about 200 mg or about 210 mg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

In one embodiment, there is provided an oligonucleotide for use according to the invention, wherein the patient is NUC-suppressed, wherein the method comprises the administration of a single dose of the oligonucleotide in an amount of about 360 mg or about 400 mg. The method may further comprises the administration of an antiviral agent, preferably a nucleos(t)ide analogue (NUC), wherein the antiviral agent is administered sequentially to the oligonucleotide, preferably wherein the period of time between the administration of the oligonucleotide and the antiviral agent is about 12 weeks.

### Hepatitis B Virus

In one embodiment, there is provided an oligonucleotide for use according to invention, wherein the hepatitis B virus is selected from any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).

### Function

There is provided an oligonucleotide for use according to the invention, wherein the administration of the oligonucleotide provides clinical benefit as measured by one or more of the following:
(a) reduction in HBsAg level, preferably at least 1 log reduction;
(b) at least 1 log reduction in HBsAg level as determined at least 50 days following the initial dose;
(c) reduction in HBV DNA level, preferably at least 2 log reduction;
(d) at least 2 log reduction in HBV DNA level as determined at least 25 days following the initial dose;
(e) reduction in HBV DNA level by 90%;
(f) reduction in HBcrAg level, preferably at least 1 log reduction;
(g) at least 1 log reduction in HBcrAg level as determined at least 25 days following the initial dose;
(h) reduction in HBeAg level, preferably at least 1 log reduction;
(i) at least 1 log reduction in HBcrAg level as determined at least 50 days following the initial dose;
(j) the presence of HBV antigen is sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems;
(k) the presence of HBV antigen is sufficiently reduced to result in PHBV, defined as serum HBeAg absence plus serum HBeAb presence if monitoring HBeAg as the determinant for seroconversion, or defined as serum HBsAg absence if monitoring HBsAg as the determinant for PHBV, as determined by currently available detection limits of commercial ELISA systems;
(l) at least three-fold increase in ALT level;
(m) at least three-fold increase in ALT level as determined at between about 20 and about 70 days following the initial dose;
(n) induction of a host-mediated, cell-mediated immune response, such as a T-cell response;
(o) substantially no significant change in the level of albumin and bilirubin, as determined at any point following the initial dose;
(p) lack of patient rebound.

The (a) reduction in HBsAg level may be at least 1, 1.5, 2, 2.5, 3, 3.5, 4, 4.5 or at least 5 log reduction. The log reduction in HBsAg level may be determined at least 25, 30, 35, 40, 45, 50, 55, 60, 65, 70, 75, 80, 85, 90, 95, 100, 150, or at least 200 days following the initial dose.

The (c) reduction in HBV DNA level may be at least 1, 1.5, 2, 2.5, 3, 3.5, 4, 4.5 or at least 5 log reduction. The log reduction in HBV DNA level may be determined at least 25, 30, 35, 40, 45, 50, 55, 60, 65, 70, 75, 80, 85, 90, 95, 100, 150, or at least 200 days following the initial dose.

The (e) reduction in HBV DNA level may be by 90%, 91, 92, 93, 94, 95, 96, 97, 98, 99%, 100% or beyond the detection limit of the assay.

The (f) reduction in HBcrAg level may be at least 1, 1.5, 2, 2.5, 3, 3.5, 4, 4.5 or at least 5 log reduction. The log reduction in HBcrAg level may be determined at least 25, 30, 35, 40, 45, 50, 55, 60, 65, 70, 75, 80, 85, 90, 95, 100, 150, or at least 200 days following the initial dose.

The (h) reduction in HBeAg level may be at least 1, 1.5, 2, 2.5, 3, 3.5, 4, 4.5 or at least 5 log reduction. The log reduction in HBeAg level may be determined at least 25, 30, 35, 40, 45, 50, 55, 60, 65, 70, 75, 80, 85, 90, 95, 100, 150, or at least 200 days following the initial dose.

The use of the oligonucleotide of the invention initiated a PHBV flare in 60% of the NUC-naïve patents who received the RNAi oligonucleotide as a single dose monotherapy. The occurrence of ‘flares’ following treatment in our study in 3 out of 5 patients who received the oligonucleotide of the current invention as a single injection was observed.

Patient rebound is a well-known term in the art that relates to the production of increased negative symptoms when the effect of a drug has passed. If a drug produces a rebound effect, the condition it was used to treat may come back even stronger when the drug is discontinued or loses effectiveness. The patient may show a 1 log decrease in a measured parameter from baseline (for example HBsAg) and rebound is defined by the measured parameter subsequently increasing back towards baseline and passing the 1 log reduction point.

### Goal of Treatment

Although the three primary HBV proteins (HBsAg, HBeAg and HBcAg) all have immunoinhibitory properties, HBsAg comprises the overwhelming majority of HBV protein in the circulation of HBV infected subjects. Additionally, while the removal (via seroconversion) of HBeAg or reductions in serum viremia are not correlated with the development of sustained control of HBV infection off treatment, the removal of serum HBsAg from the blood (and seroconversion) in HBV infection is a well-recognized prognostic indicator of antiviral response on treatment which will lead to control of HBV infection off treatment (although this only occurs in a small fraction of patients receiving immunotherapy). Thus, while reduction of all three major HBV proteins (HBsAg, HBeAg and HBcAg) may result in the optimal removal of inhibitory effect, the removal of HBsAg alone is likely sufficient in and of itself to remove the bulk of the viral inhibition of immune function in subjects with HBV infection.

Therefore, in the absence of any current treatment regimen which can restore immunological control of HBV in a large proportion of patients, there is a need for an effective treatment against HBV infection which can inhibit viral replication as well as restore immunological control in the majority of patients. Accordingly, there is a need in the art for alternative therapies and combination therapies for subjects infected with HBV and/or having an HBV-associated disease.

The invention may provide a practical method to treat patients with chronic hepatitis B virus infection with the overall goal to reduce the HBsAg level and increase the likelihood of achieving a functional or sterilizing cure. This may be achieved by using HBVS-219 as monotherapy in otherwise treatment-naïve patients, or as monotherapy run-in phase in patients naïve to any other hepatitis B treatment.

Provided is an oligonucleotide for use according to the invention, wherein the treatment may cure the patient or provide a functional cure. The term “cure” is defined as the patient no longer suffering the disease and no longer requiring additional treatment for the disease such that they are no longer a patient. The term “functional cure” is defined as an HBsAg loss of treatment response with a finite treatment regimen. The finite treatment regimen may be in accordance with any treatment regimen disclosed herein, for example a monotherapy or combination therapy in an NUC-suppressed or NUC-naïve patient.

### III. Oligonucleotide-Based Inhibitors

### Active Compound

The oligonucleotide for use according to the invention may be an oligonucleotide duplex comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
   2′-fluoro modified nucleotides at positions 3, 8-10, 12, 13, and 17,
   2′-O-methyl modified nucleotides at positions 1, 2, 4-7, 11, 14-16, 18-26, and 31-36, and one phosphorothioate linkage between the nucleotides at positions 1 and 2,
   wherein each of the nucleotides of the -GAAA- sequence on the sense strand is conjugated to a monovalent GalNAc moiety; wherein the -GAAA- sequence comprises the structure:

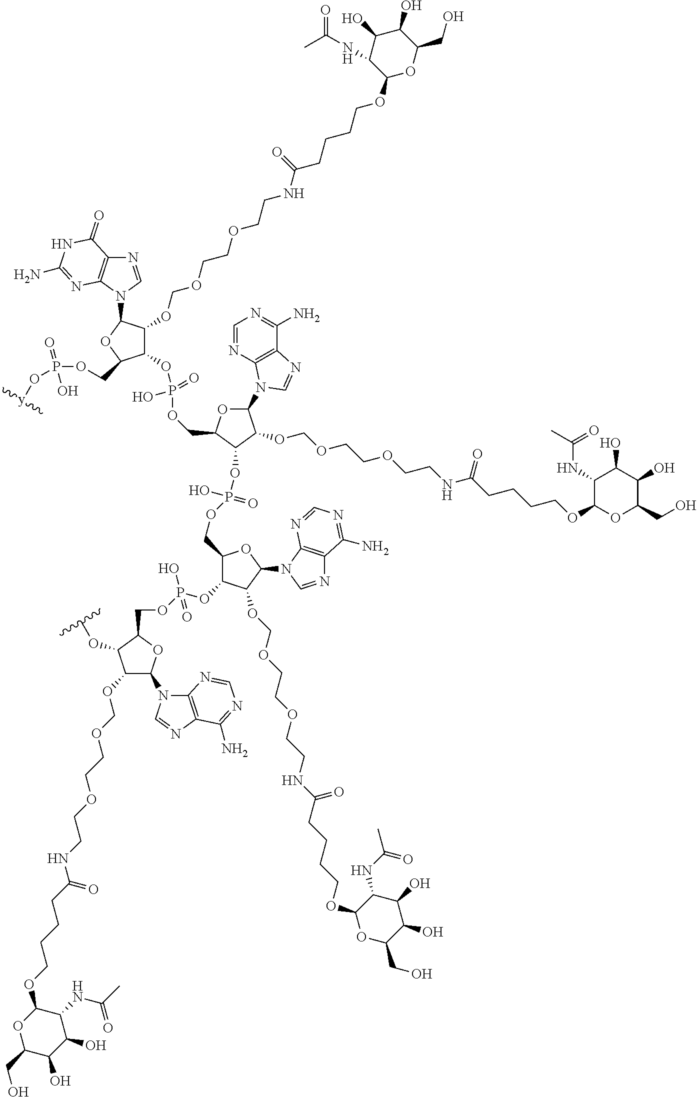

and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and five phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 5′-nucleotide of the antisense strand has the following structure:

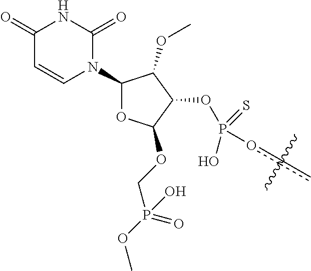

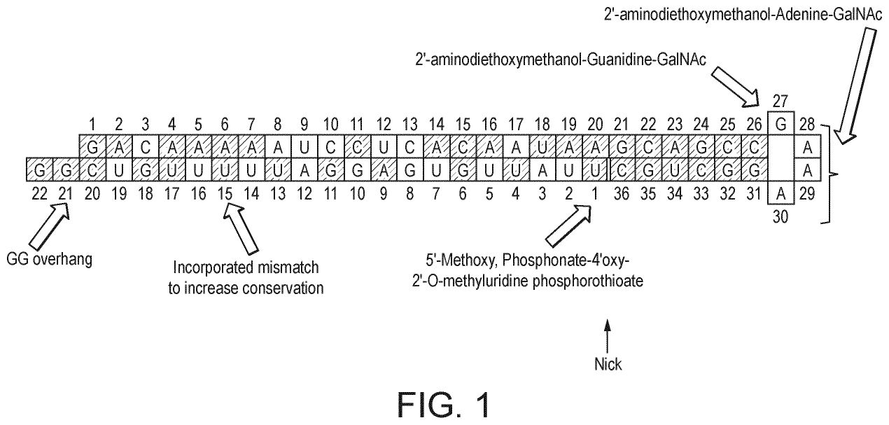

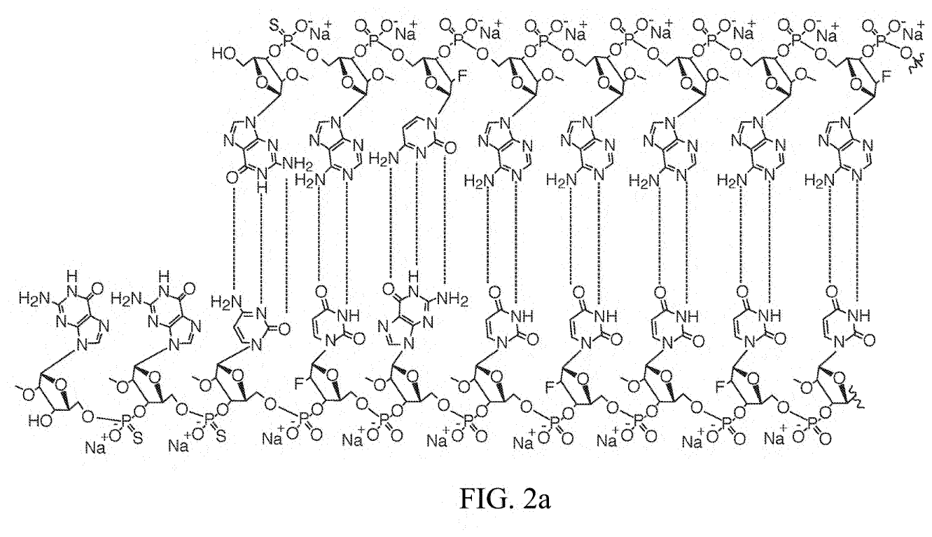

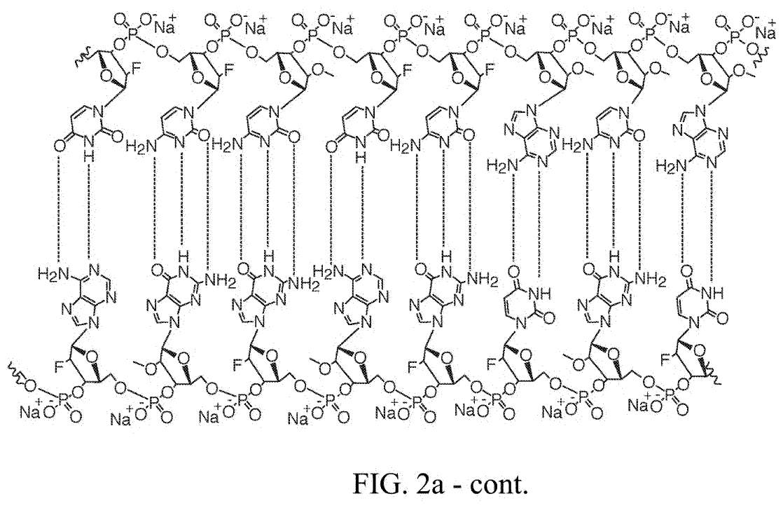

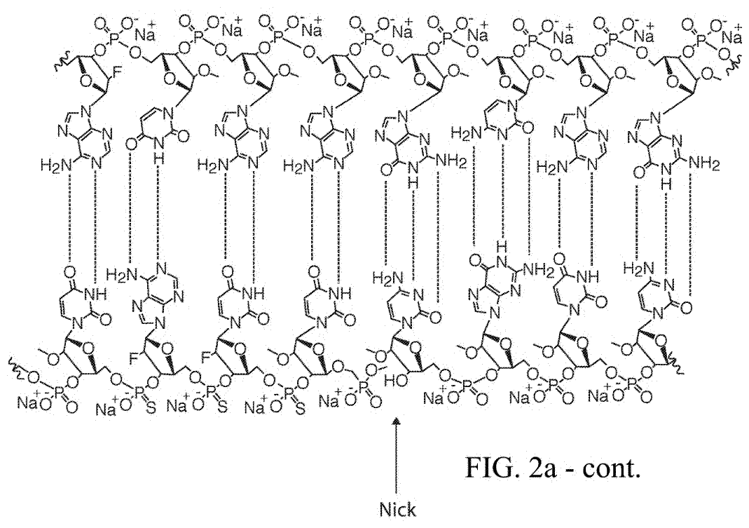

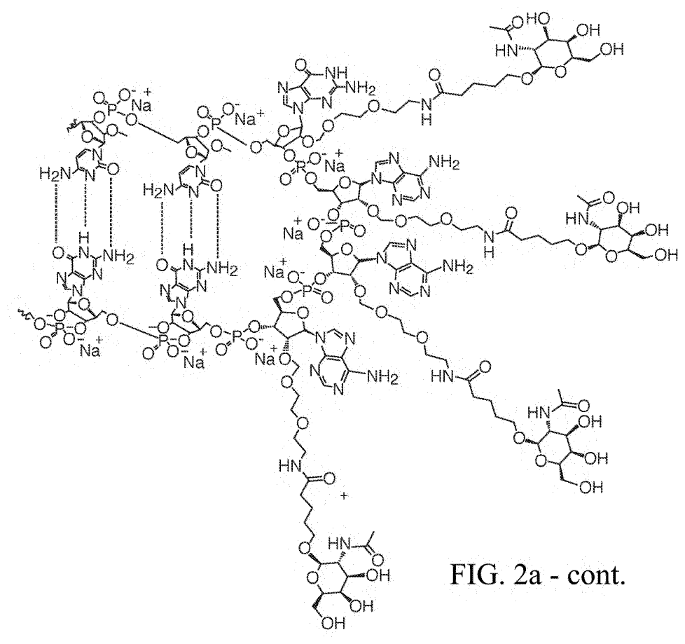

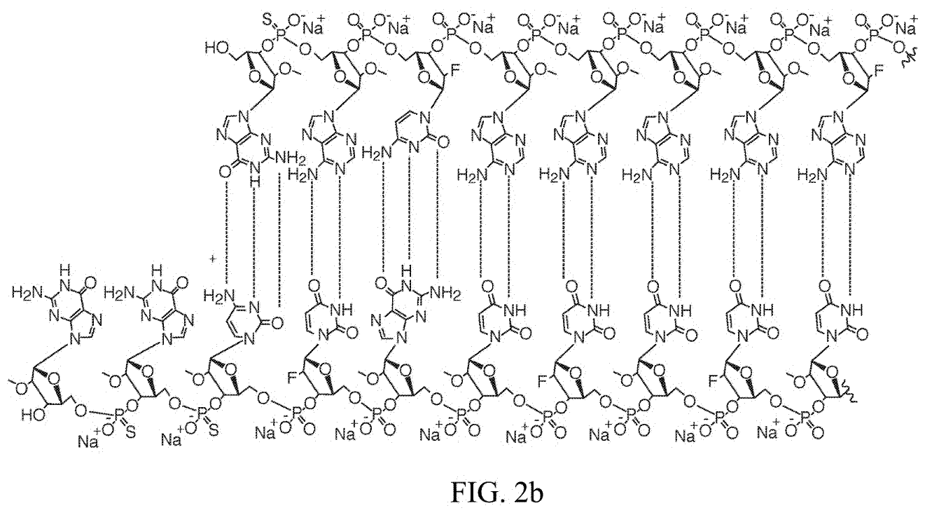

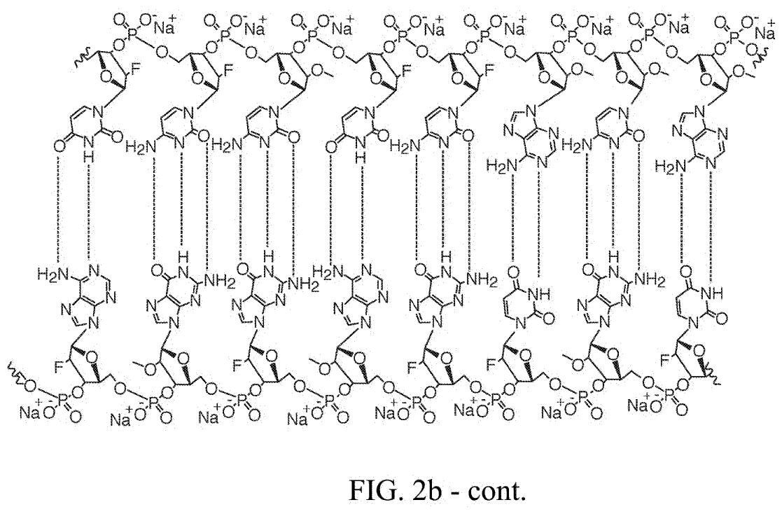

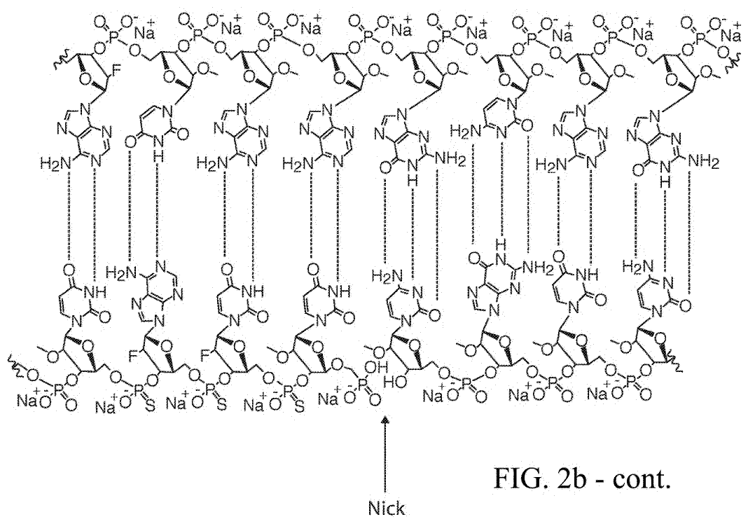

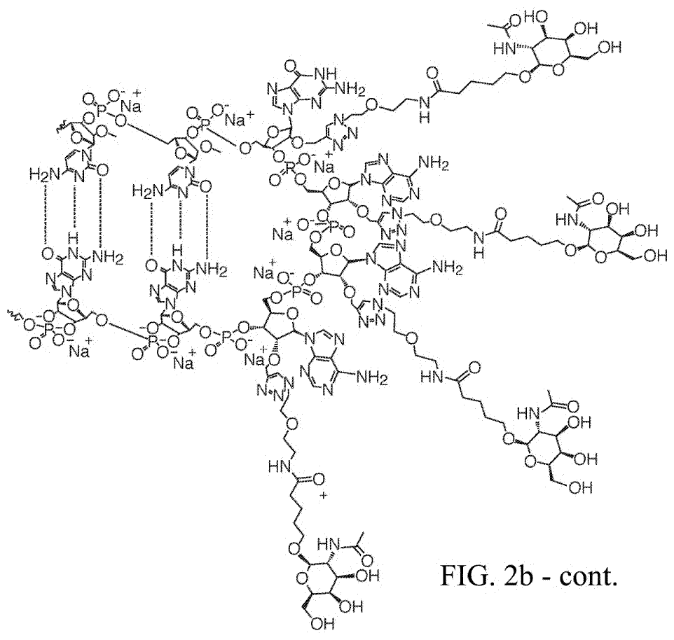

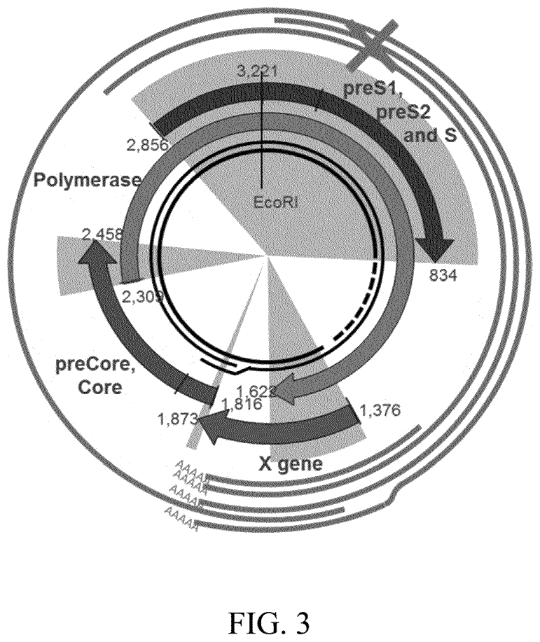

or a pharmaceutically acceptable salt thereof.
The oligonucleotide for use according to the invention may be that shown in FIG. 1 or FIG. 2A.

### HBV Surface Antigen Targeting

The oligonucleotides for use according to the invention may be used to achieve a therapeutic benefit such as a PHBV. Such oligonucleotides may result in more than 90% reduction of HBV pre-genomic RNA (pgRNA) and HBsAg mRNAs in liver. The reduction in HBsAg expression may persist for an extended period of time following a single dose or treatment regimen.

Accordingly, oligonucleotides for use provided herein are designed so as to have regions of complementarity to HBsAg mRNA for purposes of targeting the transcripts in cells and inhibiting their expression.

### Double-Stranded Oligonucleotides

There are a variety of structures of oligonucleotides that are useful for targeting HBsAg mRNA expression, including RNAi, antisense, miRNA, etc. Any of the structures described herein or elsewhere may be used as a framework to incorporate or target a sequence described herein. Double-stranded oligonucleotides for targeting HBV antigen expression (e.g., via the RNAi pathway) generally have a sense strand and an antisense strand that form a duplex with one another.

Double-stranded oligonucleotides for reducing the expression of HBsAg mRNA expression according to the invention may engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions (on one or both sides of the molecule) as well as double-stranded extensions.

Oligonucleotides provided herein according to the invention may be cleavable by Dicer enzymes. Such oligonucleotides may have an overhang (e.g., of 1, 2, or 3 nucleotides in length) in the 3′ end of the sense strand. Such oligonucleotides (e.g., siRNAs) may comprise a 21 nucleotide guide strand that is antisense to a target RNA and a complementary passenger strand, in which both strands anneal to form a 19-bp duplex and 2 nucleotide overhangs at either or both 3′ ends. Longer oligonucleotide designs are also available including oligonucleotides having a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. See, for example, U.S. Pat. Nos. 9,012,138; 9,012,621; and 9,193,753, each of which are incorporated herein for their relevant disclosures.

Other oligonucleotides disclosed herein include: 16-mer siRNAs (see, e.g., NUCLEIC ACIDS IN CHEMISTRY AND BIOLOGY. Blackburn (ed.), Royal Society of Chemistry, 2006), shRNAs (e.g., having 19 bp or shorter stems; see, e.g., Moore et al. METHODS MOL. BIOL. 2010; 629:141-58), blunt siRNAs (e.g., of 19 bps in length; see: e.g., Kraynack and Baker, R N A Vol. 12, p 163-76 (2006)), asymmetrical siRNAs (aiRNA; see, e.g., Sun et al., NAT. BIOTECHNOL. 26, 1379-82 (2008)), asymmetric shorter-duplex siRNA (see, e.g., Chang et al., MOL THER. 2009 April; 17(4): 725-32), fork siRNAs (see, e.g., Hohjoh, FEBS LETTERS, Vol 557, issues 1-3; January 2004, p 193-98), single-stranded siRNAs (Elsner; NATURE BIOTECHNOLOGY 30, 1063 (2012)), dumbbell-shaped circular siRNAs (see, e.g., Abe et al. J AM CHEM SOC 129: 15108-09 (2007)), and small internally segmented interfering RNA (sisiRNA; see, e.g., Bramsen et al., NUCLEIC ACIDS RES. 2007 September; 35(17): 5886-97). Each of the foregoing references is incorporated by reference in its entirety for the related disclosures therein. Further non-limiting examples of an oligonucleotide structures that may be used to reduce or inhibit the expression of HBsAg are microRNA (miRNA), short hairpin RNA (shRNA), and short siRNA (see, e.g., Hamilton et al., EMBO J., 2002, 21(17): 4671-79; see also, U.S. Application No. 20090099115).

### Antisense Strands

An antisense strand of an oligonucleotide may be referred to as a “guide strand.” For example, if an antisense strand can engage with RNA-induced silencing complex (RISC) and bind to an Argonaute protein, or engage with or bind to one or more similar factors, and direct silencing of a target gene, it may be referred to as a guide strand. A sense strand complementary with a guide strand may be referred to as a “passenger strand.”

### Oligonucleotide Ends

Typically, an oligonucleotide for RNAi has a two-nucleotide overhang on the 3′ end of the antisense (guide) strand. However, other overhangs are possible.

### Mismatches

An oligonucleotide may have one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. The 3′-terminus of the sense strand may contain one or more mismatches. There may be two mismatches are incorporated at the 3′ terminus of the sense strand. Base mismatches, or destabilization of segments at the 3′-end of the sense strand of the oligonucleotide may improve the potency of synthetic duplexes in RNAi, possibly through facilitating processing by Dicer.

An antisense strand may have a region of complementarity to an HBsAg transcript that contains one or more mismatches compared with a corresponding transcript sequence. A region of complementarity on an oligonucleotide may have up to 1, up to 2, up to 3, up to 4, up to 5, etc. mismatches provided that it maintains the ability to form complementary base pairs with the transcript under appropriate hybridization conditions. Alternatively, a region of complementarity of an oligonucleotide may have no more than 1, no more than 2, no more than 3, no more than 4, or no more than 5 mismatches provided that it maintains the ability to form complementary base pairs with HBsAg mRNA under appropriate hybridization conditions. If there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with HBsAg mRNA under appropriate hybridization conditions.

### Single-Stranded Oligonucleotides

Recent efforts have demonstrated the activity of single-stranded RNAi oligonucleotides (see, e.g., Matsui et al. (May 2016), MOLECULAR THERAPY, Vol. 24(5), 946-55). Further, antisense molecules have been used for decades to reduce expression of specific target genes (see, e.g., Bennett et al.; PHARMACOLOGY OF ANTISENSE DRUGS, ANNUAL REVIEW OF PHARMACOLOGY AND TOXICOLOGY, Vol. 57: 81-105).

### Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., NUCLEIC ACIDS RES., 2009, 37, 2867-81; Bramsen and Kjems (FRONTIERS IN GENETICS, 3 (2012): 1-22).

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of its nucleotides to be modified.

### Sugar Modifications

A modified sugar (also referred to herein is a sugar analog) may include non-natural alternative carbon structures such as those present in locked nucleic acids (“LNA”) (see, e.g., Koshkin et al. (1998), TETRAHEDRON 54, 3607-3630), unlocked nucleic acids (“UNA”) (see, e.g., Snead et al. (2013), MOLECULAR THERAPY—NUCLEIC ACIDS, 2, e103), and bridged nucleic acids (“BNA”) (see, e.g., Imanishi and Obika (2002), The Royal Society of Chemistry, CHEM. COMMUN., 1653-59). Koshkin et al., Snead et al., and Imanishi and Obika are incorporated by reference herein for their disclosures relating to sugar modifications.

A nucleotide modification in a sugar mat comprise a 2′-modification. A 2′-modification may be 2′-aminoethyl, 2′-fluoro, 2′-O-methyl, 2′-O-methoxyethyl, and 2′-deoxy-2′-fluoro-β-d-arabinonucleic acid. Typically, the modification is 2′-fluoro, 2′-O-methyl, or 2′-O-methoxyethyl. A modification in a sugar may comprise a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. A modified nucleotide may have an acyclic sugar that lacks a 2′-carbon to 3′-carbon bond. A modified nucleotide may have a thiol group, e.g., in the 4′ position of the sugar.

The terminal 3′-end group (e.g., a 3′-hydroxyl) may be a phosphate group or other group, which can be used, for example, to attach linkers, adapters or labels or for the direct ligation of an oligonucleotide to another nucleic acid.

### 5′ Terminal Phosphates

5′-terminal phosphate groups of oligonucleotides may enhance the interaction with Argonaut 2. However, oligonucleotides comprising a 5′-phosphate group may be susceptible to degradation via phosphatases or other enzymes, which can limit their bioavailability in vivo. Oligonucleotides may include analogs of 5′ phosphates that are resistant to such degradation. A phosphate analog may be oxymethylphosphonate, vinylphosphonate, or malonyl phosphonate. The 5′ end of an oligonucleotide strand is attached to a chemical moiety that mimics the electrostatic and steric properties of a natural 5′-phosphate group (“phosphate mimic”) (see, e.g., Prakash et al. (2015), NUCLEIC ACIDS RES., NUCLEIC ACIDS REs. 2015 Mar. 31; 43(6): 2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). A hydroxyl group may be attached to the 5′ end of the oligonucleotide.

An oligonucleotide may have a phosphate analog at a 4′-carbon position of the sugar (referred to as a “4′-phosphate analog”). See, for example, WO 2018/045317, and WO 2018/045317, the contents of each of which relating to phosphate analogs are incorporated herein by reference. An oligonucleotide may comprise a 4′-phosphate analog at a 5′-terminal nucleotide. A phosphate analog is may be oxymethylphosphonate, in which the oxygen atom of the oxymethyl group is bound to the sugar moiety (e.g., at its 4′-carbon) or analog thereof. A 4′-phosphate analog is a thiomethyl phosphonate or an aminomethyl phosphonate, in which the sulfur atom of the thiomethyl group or the nitrogen atom of the aminomethyl group is bound to the 4′-carbon of the sugar moiety or analog thereof. A 4′-phosphate analog is an oxymethylphosphonate. An oxymethylphosphonate may be represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. The alkyl group may be CH₂CH₃. More typically, R is independently selected from H, CH₃, or CH₂CH₃.

The phosphate analog attached to the oligonucleotide according to the invention is a 5′ mono-methyl protected MOP. In some embodiments, the following uridine nucleotide comprising a phosphate analog may be used, e.g., at the first position of a guide (antisense) strand:

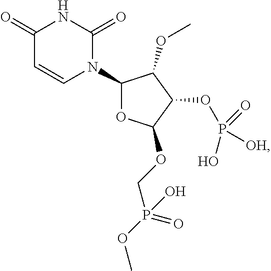 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′oxy-2′-O-methyluridine.

### Modified Internucleoside Linkages

A modified internucleotide linkage may be a phosphorothioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. At least one modified internucleotide linkage of any one of the oligonucleotides as disclosed herein is a phosphorothioate linkage.

### Base Modifications

The oligonucleotides provided herein have one or more modified nucleobases. Modified nucleobases (also referred to herein as base analogs) may be linked at the 1′ position of a nucleotide sugar moiety. A modified nucleobase may be a nitrogenous base. A modified nucleobase may not contain a nitrogen atom. See e.g., U.S. Published Patent Application No. 20080274462. A modified nucleotide may comprise a universal base. However, a modified nucleotide may not contain a nucleobase (abasic).

A universal base may be a heterocyclic moiety located at the 1′ position of a nucleotide sugar moiety in a modified nucleotide, or the equivalent position in a nucleotide sugar moiety substitution that, when present in a duplex, can be positioned opposite more than one type of base without substantially altering the structure of the duplex. Compared to a reference single-stranded nucleic acid (e.g., oligonucleotide) that is fully complementary to a target nucleic acid, a single-stranded nucleic acid containing a universal base may form a duplex with the target nucleic acid that has a lower Tₘ than a duplex formed with the complementary nucleic acid. However, compared to a reference single-stranded nucleic acid in which the universal base has been replaced with a base to generate a single mismatch, the single-stranded nucleic acid containing the universal base may form a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. NUCLEIC ACIDS RES. 1995 Nov. 11; 23(21):4363-70; Loakes et al., 3-Nitropyrrole and 5-nitroindole as universal bases in primers for DNA sequencing and PCR, NUCLEIC ACIDS RES. 1995 Jul. 11; 23(13):2361-66; Loakes and Brown, 5-Nitroindole as an universal base analogue, NUCLEIC ACIDS RES. 1994 Oct. 11; 22(20):4039-43. Each of the foregoing is incorporated by reference herein for their disclosures relating to base modifications).

### Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

A reversibly modified nucleotide may comprise a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”), PCT Publication No. WO 2015/188197 to Solstice Biologics, Ltd. (“Solstice”), Meade et al., NATURE BIOTECHNOLOGY, 2014, 32:1256-63 (“Meade”), PCT Publication No. WO 2014/088920 to Merck Sharp & Dohme Corp, each of which are incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. AM. CHEM. SOC. 2003, 125:940-50).

A reversible modification may allow protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. The structure of the glutathione-sensitive moiety may be engineered to modify the kinetics of its release.

A glutathione-sensitive moiety may be attached to the sugar of the nucleotide. A glutathione-sensitive moiety may be attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. The glutathione-sensitive moiety may be located at the 3′-carbon of a sugar, particularly when the modified nucleotide is the 3′-terminal nucleotide of the oligonucleotide. The glutathione-sensitive moiety may comprise a sulfonyl group. See, e.g., U.S. Prov. Appl. No. 62/378,635, entitled Compositions Comprising Reversibly Modified Oligonucleotides and Uses Thereof, which was filed on Aug. 23, 2016, the contents of which are incorporated by reference herein for its relevant disclosures.

### Targeting Ligands

It may be desirable to target the oligonucleotides of the disclosure to one or more cells or one or more organs. Such a strategy may help to avoid undesirable effects in other organs or may avoid undue loss of the oligonucleotide to cells, tissue or organs that would not benefit for the oligonucleotide. Oligonucleotides disclosed herein may be modified to facilitate targeting of a particular tissue, cell or organ, e.g., to facilitate delivery of the oligonucleotide to the liver. Oligonucleotides disclosed herein may be modified to facilitate delivery of the oligonucleotide to the hepatocytes of the liver. An oligonucleotide may comprise a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. A targeting ligand may be an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells. The targeting ligand may be one or more GalNAc moieties.

1 or more (e.g., 1, 2, 3, 4, 5 or 6) nucleotides of an oligonucleotide may each be conjugated to a separate targeting ligand. 2 to 4 nucleotides of an oligonucleotide may each conjugated to a separate targeting ligand. Targeting ligands may be conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand.

It may be desirable to target an oligonucleotide that reduces the expression of HBV antigen to the hepatocytes of the liver of a subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetyl galactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

An oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand, where each GalNAc moiety is conjugated to one nucleotide.

In some embodiments, an oligonucleotide herein comprises a monovalent GalNAc attached to a Guanidine nucleotide, referred to as [ademg-GalNAc] or 2′-aminodiethoxymethanol-Guanidine-GalNAc, as depicted below:

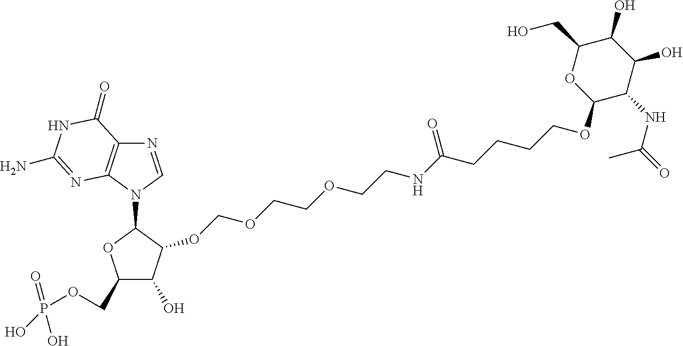 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

The oligonucleotide for use according to the invention comprises a monovalent GalNAc attached to an adenine nucleotide, referred to as [ademA-GalNAc] or 2′-aminodiethoxymethanol-Adenine-GalNAc, as depicted below.

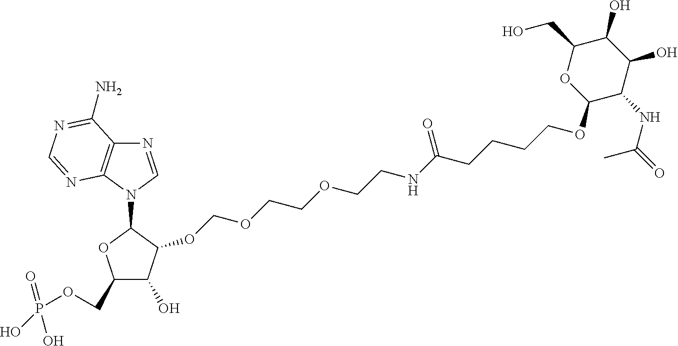

An example of such conjugation is shown below for a loop comprising from 5′ to 3′ the nucleotide sequence GAAA (L=linker, X=heteroatom) stem attachment points are shown. Such a loop may be present, for example, at positions 27-30 of the molecule shown in FIG. 1. In the chemical formula,

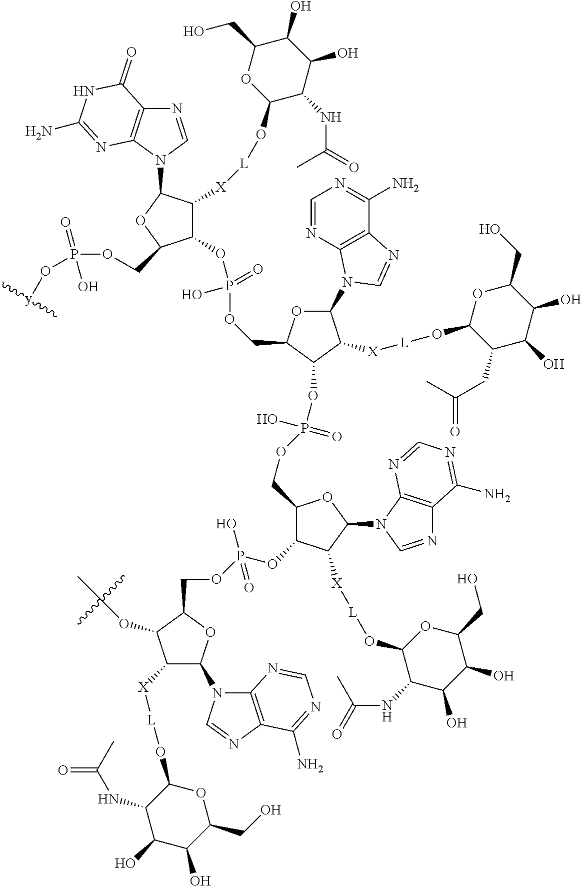

is an attachment point to the oligonucleotide strand.

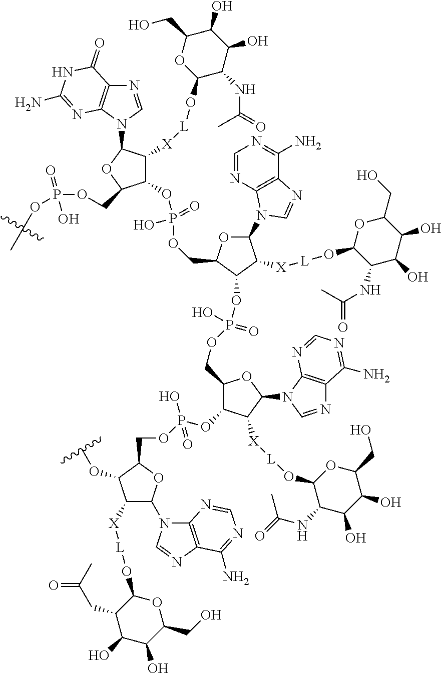

Appropriate methods or chemistry (e.g., click chemistry) can be used to link a targeting ligand to a nucleotide. A targeting ligand may be conjugated to a nucleotide using a click linker. An acetal-based linker may be used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which relating to such linkers are incorporated herein by reference. The linker may be a labile linker. However, the linker may be stable.

An example is shown below for a loop comprising from 5′ to 3′ the nucleotides GAAA, in which GalNAc moieties are attached to nucleotides of the loop using an acetal linker. Such a loop may be present, for example, at positions 27-30 of the molecule shown in FIG. 1. In the chemical formula,

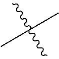

is an attachment point to the oligonucleotide strand.

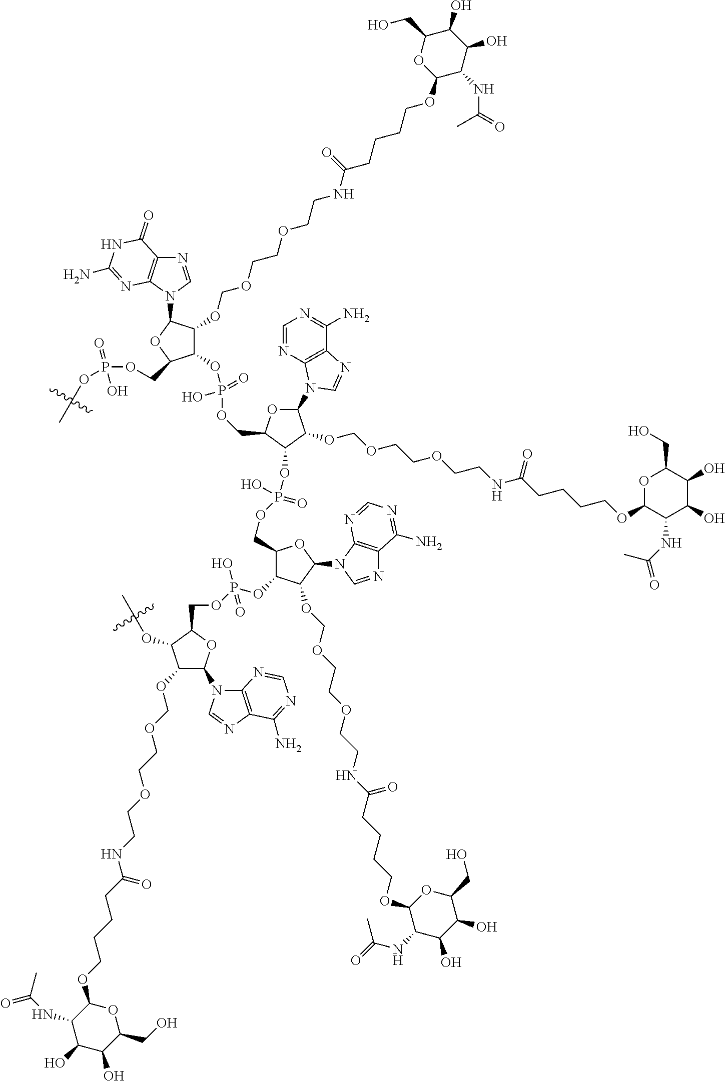

### Recognition and Management of Alanine Aminotransferase Flares

Close patient monitoring is important and includes the need for unscheduled intercurrent diagnostic evaluations as needed, for any participant who fits the following flare definition: as a substantial alanine aminotransferase (ALT) elevation that is greater than 3-fold above the participant's baseline ALT value or greater than 3-fold above post-baseline nadir value (whichever value is lower), with an absolute ALT value that is at least at least 7×ULN, such as at least 10×ULN.

A confirmed rise in a participant's serum ALT to a value that is greater than 3-fold above the participant's baseline ALT value or greater than 3-fold above post-baseline nadir value (whichever value is lower), with an absolute ALT value that is at least at least 7×ULN, such as at least 10×ULN. Upon laboratory evidence of an ALT increase (flare) in a study participant, participant management should include a prompt clinic visit and further follow-up visits as needed.

The participant should be checked for or consistently monitored for serum albumin and direct bilirubin levels, to determine if liver functions (synthetic and excretory) are stable or deteriorating.

The participant may be evaluated for potential intercurrent causes of the ALT elevation, (e.g., hepatitis A infection (HAV), Hepatitis E infection (HEV), or other infection; toxin exposures; hepatotoxic herbal supplements or concomitant medicines).

The participant's recent HBV DNA levels may be checked for changes in levels. If HBV DNA is declining, the ALT increase (flare) is presumptively not due to a viral breakthrough (resistance) NHBV flare and could be a ‘beneficial’ or PHBV flare if no intervening causes are found.

Study treatment interruption (pending diagnostic tests) or discontinuation is recommended for an ALT increase (flare) with biochemical evidence of hepatic decompensation. That is an ALT increase (flare) that is temporally associated with one or both of the following concurrent laboratory findings:
i. a confirmed direct bilirubin elevation >2×Baseline and >2×ULN; or
ii. a confirmed decrease in serum albumin level of 0.5 g/dL or more.

### Biomarkers

There is provided an oligonucleotide for use according to the invention, wherein the method further comprises the step of determining the level of a biomarker, for example HBsAg, HBeAg, HbcrAg or ALT, in a sample obtained from the patient. The determining step may be carried out during a treatment holiday. The step of administering one or more further doses of the oligonucleotide may follow the determining step. The need to re-administer the oligonucleotide of the invention or re-commence treatment may depend on the level of aforementioned biomarker determined. Treatment may be ceased. Treatment may be recommenced.

A major unmet need in the management of chronic HBV patients is the definition of biomarkers that can predict the safe discontinuation of NUC therapy. There is no consensus on current treatment guidelines on the optimal time to consider stopping NUC therapy. Seroconversion of the surface antigen of HBV (HBsAg) or HBsAg to values below 100 IU/ml (7) in HBV e-antigen-negative (HBeAg-negative) patients is recommended by some as a safe stopping point; however, such values are observed in a minority of NUC-treated patients. The recent FINITE study of stopping NUC therapy in HBeAg patients reports that 13 out of 21 patients were able to remain off therapy for nearly 3 years, yet no criteria that distinguishes which patients can safely discontinue therapy has been identified. According to the current invention, antiviral immunity plays a critical role in the suppression and control of HBV infection and therefore we developed immunological biomarkers to predict when NUC monotherapy can be safely discontinued.

Currently, the clinical management of chronic hepatitis B virus (HBV) patients is based exclusively on virological parameters that cannot independently determine in which patients nucleos(t)ide-analogue (NUC) therapy can be safely discontinued. NUCs efficiently suppress viral replication, but do not eliminate HBV. Thus, therapy discontinuation can be associated with virological and biochemical relapse and, consequently, therapy in the majority is life-long.

Antiviral immunity is pivotal for HBV control, to that end the development and recognition of certain biomarkers for the determination of safe discontinuation of NUCs or other HBV treatment regimens is critical for determining when therapy can be stopped or removed.

Therefore, according to the current invention we look to the beneficial role of the expression of exhaustion markers on T cells during chronic viral infection since PD-1 expression associates with providing a correct assessment on the long-term persistence of virus-specific T cells in human chronic viral infection. Therefore, the current invention provides that the levels of residual HBV-specific T cells present in patients during NUC therapy can be used to predict the safe discontinuation of NUC antiviral therapy.

### IV. Formulations

The oligonucleotide for use according to the invention may be in the form of a pharmaceutically acceptable salt or pharmaceutical composition.

The oligonucleotide for use according to the invention may be in the form of a pharmaceutically acceptable salt. The pharmaceutically acceptable salt may be a sodium salt. The pharmaceutically acceptable salt may be a potassium salt. The pharmaceutically acceptable salt of the oligonucleotide may be as shown in FIG. 2A or 2B, preferably FIG. 2A.

Provided is a pharmaceutical composition that may comprise the oligonucleotide or pharmaceutically acceptable salt thereof of according to the invention and a pharmaceutically acceptable solvent, carrier, excipient, diluent or adjuvant for use according to the invention. The pharmaceutically acceptable solvent, carrier, excipient, diluent or adjuvant may comprise saline. The saline may be phosphate buffered saline. Preferably, the oligonucleotide is formulated in phosphate-buffered saline. The pharmaceutically acceptable solvent, carrier, excipient, diluent or adjuvant may be water, for example water for injection.

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation.

Provided herein are oligonucleotides for use according to the invention that may reduce the expression of HBV antigen (e.g., HBsAg). The present invention may provide a pharmaceutical composition comprising an oligonucleotide as described herein, and a pharmaceutically acceptable excipient. Such compositions can be suitably formulated such that when administered to a subject, either into the immediate environment of a target cell or systemically, a sufficient portion of the oligonucleotides enter the cell to reduce HBV antigen expression. Any of a variety of suitable oligonucleotide formulations can be used to deliver oligonucleotides for the reduction of HBV antigen as disclosed herein. The oligonucleotide for use according to the invention may be formulated in buffer solutions such as phosphate-buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

A pharmaceutically acceptable excipient may be buffer solutions, such as phosphate-buffered saline solutions, liposomes, micellar structures, and capsids.

A formulation may comprise a lipid nanoparticle. A pharmaceutically acceptable excipient may comprise a liposome, a lipid, a lipid complex, a microsphere, a microparticle, a nanosphere, or a nanoparticle, or may be otherwise formulated for administration to the cells, tissues, organs, or body of a subject in need thereof (see, e.g., REMINGTON: THE SCIENCE AND PRACTICE OF PHARMACY, 22ND EDITION, PHARMACEUTICAL PRESS, 2013).

Formulations as disclosed herein may comprise a pharmaceutically acceptable excipient. A pharmaceutically acceptable excipient may confer to a composition improved stability, improved absorption, improved solubility and/or therapeutic enhancement of the active ingredient. Aa pharmaceutically acceptable excipient may be a buffering agent (e.g., sodium citrate, sodium phosphate, a tris base, or sodium hydroxide) or a vehicle (e.g., a buffered solution, petrolatum, dimethyl sulfoxide, or mineral oil). An oligonucleotide for use may be lyophilized for extending its shelf-life and then made into a solution before use (e.g., administration to a subject). Accordingly, a pharmaceutically acceptable excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable pharmaceutically acceptable carriers include physiological saline, bacteriostatic water, Cremophor EL™ (BASF, Parsippany, N.J.) or phosphate buffered saline (PBS). The pharmaceutically acceptable carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

A composition may contain at least about 0.1% of the therapeutic agent (e.g., an oligonucleotide for reducing HBV antigen expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though the oligonucleotides for use according to the invention may be directed to liver-targeted delivery of any of the oligonucleotides disclosed herein, targeting of other tissues is also contemplated.

### V. Methods of Use

### Reducing HBsAg Expression

In some embodiments, methods are provided for delivering to a cell an effective amount of any one of oligonucleotides for use according to the invention for purposes of reducing expression of HBsAg. Methods provided herein are useful in any appropriate cell type. A cell may be any cell that expresses HBV antigen (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). The cell may be a primary cell that has been obtained from a subject and that may have undergone a limited number of a passages, such that the cell substantially maintains its natural phenotypic properties. A cell to which the oligonucleotide is delivered may be ex vivo or in vitro (i.e., can be delivered to a cell in culture or to an organism in which the cell resides). Methods may also be provided for delivering to a cell an effective amount of any one of the oligonucleotides disclosed herein for purposes of reducing expression of HBsAg solely in hepatocytes.

Oligonucleotides for use according to the invention can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of HBV antigen expression (e.g., RNA, protein). The extent to which an oligonucleotide provided herein reduces levels of expression of HBV antigen may be evaluated by comparing expression levels (e.g., mRNA or protein levels) of HBV antigen to an appropriate control (e.g., a level of HBV antigen expression in a cell or population of cells to which an oligonucleotide has not been delivered or to which a negative control has been delivered). An appropriate control level of HBV antigen expression may be a predetermined level or value, such that a control level need not be measured every time. The predetermined level or value can take a variety of forms. A predetermined level or value can be single cut-off value, such as a median or mean.

Administration of an oligonucleotide for use according to the invention may result in a reduction in the level of HBV antigen (e.g., HBsAg) expression in a cell. The reduction in levels of HBV antigen expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of HBV antigen. The appropriate control level may be a level of HBV antigen expression in a cell or population of cells that has not been contacted with an oligonucleotide as described herein. The effect of delivery of an oligonucleotide to a cell according to a method disclosed herein may be assessed after a finite period of time. For example, levels of HBV antigen may be analyzed in a cell at least 8 hours, 12 hours, 18 hours, 24 hours; or at least one, two, three, four, five, six, seven, fourteen, twenty-one, twenty-eight, thirty-five, forty-two, forty-nine, fifty-six, sixty-three, seventy, seventy-seven, eighty-four, ninety-one, ninety-eight, 105, 112, 119, 126, 133, 140, or 147 days after introduction of the oligonucleotide into the cell.

The reduction in the level of HBV antigen (e.g., HBsAg) expression may persist for an extended period of time following administration. A detectable reduction in HBsAg expression may persist within a period of 7 to 70 days following administration of an oligonucleotide described herein. For example, the detectable reduction may persist within a period of 10 to 70, 10 to 60, 10 to 50, 10 to 40, 10 to 30, or 10 to 20 days following administration of the oligonucleotide. The detectable reduction may persist within a period of 20 to 70, 20 to 60, 20 to 50, 20 to 40, or 20 to 30 days following administration of the oligonucleotide. The detectable reduction may persist within a period of 30 to 70, 30 to 60, 30 to 50, or 30 to 40 days following administration of the oligonucleotide. The detectable reduction may persist within a period of 40 to 70, 40 to 60, 40 to 50, 50 to 70, 50 to 60, or 60 to 70 days following administration of the oligonucleotide.

A detectable reduction in HBsAg expression may persist within a period of 2 to 21 weeks following administration of an oligonucleotide for use as described herein. For example, the detectable reduction may persist within a period of 2 to 20, 4 to 20, 6 to 20, 8 to 20, 10 to 20, 12 to 20, 14 to 20, 16 to 20, or 18 to 20 weeks following administration of the oligonucleotide. The detectable reduction may persist within a period of 2 to 16, 4 to 16, 6 to 16, 8 to 16, 10 to 16, 12 to 16, or 14 to 16 weeks following administration of the oligonucleotide. The detectable reduction may persist within a period of 2 to 12, 4 to 12, 6 to 12, 8 to 12, or 10 to 12 weeks following administration of the oligonucleotide. The detectable reduction may persist within a period of 2 to 10, 4 to 10, 6 to 10, or 8 to 10 weeks following administration of the oligonucleotide.

The oligonucleotide for use in methods of treatment may be delivered in the form of a transgene that is engineered to express in a cell the oligonucleotides (e.g., its sense and antisense strands). An oligonucleotide may be delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). Transgenes may be injected directly to a subject.

### Treatment Methods

The primary goal of treatment for HBV is to permanently suppress HBV replication and improve liver disease. Clinically important short-term goals are to achieve HBeAg-seroconversion, normalization of serum ALT and AST, resolution of liver inflammation and to prevent hepatic decompensation. The ultimate goal of treatment is to achieve durable response to prevent development of cirrhosis, liver cancer and prolong survival. According to other treatments, HBV infection cannot be eradicated completely due to persistence of a particular form of viral covalently closed circular DNA (ccc HBV DNA) in the nuclei of infected hepatocytes. However, treatment-induced clearance of serum HBsAg is a marker of termination of chronic HBV infection and has been associated with the best long-term outcome.

The current invention may relate to methods for reducing HBsAg expression (e.g., reducing HBsAg protein expression) in a subject. The methods may comprise administering to a subject in need thereof an effective amount of any one of the oligonucleotides disclosed herein. The present disclosure provides for both prophylactic and therapeutic methods of treating a subject at risk of (or susceptible to) HBV infection and/or a disease or disorder associated with HBV infection.

The present invention may provide a method for treating a hepatitis B virus infection or a disease or disorder associated with a hepatitis B virus infection in a subject, comprising administering to the subject a therapeutically effective amount of an oligonucleotide, or a pharmaceutically acceptable salt thereof, or a pharmaceutical composition thereof, as described herein.

The present invention may provide a method for promoting seroconversion in a subject infected with a hepatitis B virus, the method comprising: administering to the subject a therapeutically effective amount of an oligonucleotide, or a pharmaceutically acceptable salt thereof, or a pharmaceutical composition thereof, as described herein; and monitoring for presence of HBeAg plus HbeAb, and/or presence of HbsAg, in a serum sample of the mammal; wherein the absence of HBeAg plus the presence of HBeAb in the serum sample if monitoring HBeAg as the determinant for seroconversion, or the absence of HBsAg in the serum sample if monitoring HBsAg as the determinant for seroconversion, as determined by currently available detection limits of commercial ELISA systems, is indication of seroconversion in the mammal or the occurrence of a PHBV.

The present invention may provide a method for reducing an amount of HBV DNA in a subject infected with a hepatitis B virus, the method comprising administering to the subject a therapeutically effective amount of an oligonucleotide, or a pharmaceutically acceptable salt thereof, or a pharmaceutical composition thereof, as described herein.

The present invention may provide a method for inducing a PHBV seroconversion event against HBV, comprising administering to a subject in need thereof a therapeutically effective amount of an oligonucleotide, or a pharmaceutically acceptable salt thereof, or a pharmaceutical composition thereof, as described herein.

According to the method of treatment described herein, HBV antigen HBsAg may be reduced. HBV antigen HBeAg may be reduced. Presence of HBV antigen may be sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems.

According to the method of treatment described herein, the amount of HBV DNA may be reduced 90% compared to the amount before administration of an oligonucleotide, or a pharmaceutically acceptable salt thereof, or a pharmaceutical composition as described herein.

According to the method of treatment described herein, an HBV viral load may be reduced in a subject. An HBV viral load may be suppressed in a subject.

According to the method of treatment described herein, a subject is a NUC-naïve patient.

According to the method of treatment described herein, the subject to be treated is a human.

### VI. Numbered Embodiments

1. An oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
      wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP);
   or a pharmaceutically acceptable salt thereof,
   for use in a method for treating hepatitis B or hepatitis B virus (HBV) infection in a human patient
   said method comprising administering to the patient via the subcutaneous route an initial dose of from about 0.1 mg/kg to about 12 mg/kg of the oligonucleotide.
2. An oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
      wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP);
   or a pharmaceutically acceptable salt thereof,
   for use in a method for treating hepatitis B or hepatitis B virus (HBV) infection in a human patient
   said method comprising administering to the patient via the subcutaneous route an initial dose of from about 6 mg to about 800 mg of the oligonucleotide.
3. The oligonucleotide for use according to embodiment 1 or embodiment 2, wherein the hepatitis B or HBV infection is chronic hepatitis B or chronic HBV infection.
4. The oligonucleotide for use according to any one of embodiments 1-3, wherein the oligonucleotide is administered by subcutaneous injection.
5. The oligonucleotide for use according to any one of embodiments 1 or 3-4, wherein the initial dose is from about 0.5 mg/kg to about 10 mg/kg.
6. The oligonucleotide for use according to any one of embodiments 1 or 3-5, wherein the initial dose is from about 1.5 mg/kg to about 6 mg/kg.
7. The oligonucleotide for use according to any one of embodiments 1 or 3-6, wherein the initial dose is about 1.5 mg/kg.
8. The oligonucleotide for use according to any one of embodiments 1 or 3-6, wherein the initial dose is about 3 mg/kg.
9. The oligonucleotide for use according to any one of embodiments 1 or 3-6, wherein the initial dose is about 6 mg/kg.
10. The oligonucleotide for use according to any one of embodiments 2-4, wherein the initial dose is from about 34 mg to about 667 mg.
11. The oligonucleotide for use according to any one of embodiments 2-4 or 10, wherein the initial dose is from about 100 mg to about 400 mg.
12. The oligonucleotide for use according to any one of embodiments 2-4 or 10-11, wherein the initial dose is about 100 mg.
13. The oligonucleotide for use according to any one of embodiments 2-4 or 10-11, wherein the initial dose is about 200 mg.
14. The oligonucleotide for use according to any one of embodiments 2-4 or 10-11, wherein the initial dose is about 400 mg.
15. The oligonucleotide for use according to any one of embodiments 1-14, wherein the initial dose is a single dose or is the only dose administered.
16. The oligonucleotide for use according to any one of embodiments 1-15, wherein the method consists of or consists essentially of the administering the oligonucleotide.
17. The oligonucleotide for use according to any one of embodiments 1-16, wherein the method comprises or consists of or consists essentially of the administering the oligonucleotide, to the exclusion of other anti-HBV agents.
18. The oligonucleotide for use according to any one of embodiments 1-17, wherein the method comprises administering a single dose of the oligonucleotide.
19. The oligonucleotide for use according to any one of embodiments 1-18, wherein the method consists of or consists essentially of administering a single dose of the oligonucleotide.
20. The oligonucleotide for use according to any one of embodiments 1-19, wherein the treatment of hepatitis B or HBV infection is provided by said administering of said initial dose, said one dose or said single dose of the oligonucleotide.
21. The oligonucleotide for use according to any one of embodiments 1, 3-9 or 15-17, further comprising administering to the patient one or more subsequent doses of the oligonucleotide in an amount that is from about 0.1 mg/kg to about 12 mg/kg.
22. The oligonucleotide for use according to embodiment 21, wherein the subsequent dose(s) is from about 0.5 mg/kg to about 10 mg/kg.
23. The oligonucleotide for use according to embodiment 21 or embodiment 22, wherein the subsequent dose(s) is from about 1.5 mg/kg to about 6 mg/kg.
24. The oligonucleotide for use according to any one of embodiments 21-23, wherein the subsequent dose(s) is about 1.5 mg/kg.
25. The oligonucleotide for use according to any one of embodiments 21-23, wherein subsequent dose(s) is about 3 mg/kg.
26. The oligonucleotide for use according to any one of embodiments 21-23, wherein subsequent dose(s) is about 6 mg/kg.
27. The oligonucleotide for use according to any one of embodiments 21-26, wherein the amount of each of the initial and subsequent doses is the same or is different and is independently selected from the group consisting of: about 1.5 mg/kg, about 3 mg/kg and about 6 mg/kg.
28. The oligonucleotide for use according to any one of embodiments 2-4 or 10-17, further comprising administering to the patient one or more subsequent doses of the oligonucleotide in an amount that is from about 6 mg to about 800 mg.
29. The oligonucleotide for use according to embodiment 28, wherein the subsequent dose(s) is from about 34 mg to about 667 mg.
30. The oligonucleotide for use according to embodiment 28 or embodiment 29, wherein the subsequent dose(s) is from about 100 mg to about 400 mg.
31. The oligonucleotide for use according to any one of embodiments 28-30, wherein the subsequent dose(s) is about 100 mg.
32. The oligonucleotide for use according to any one of embodiments 28-30, wherein subsequent dose(s) is about 200 mg.
33. The oligonucleotide for use according to any one of embodiments 28-30, wherein subsequent dose(s) is about 400 mg.
34. The oligonucleotide for use according to any one of embodiments 28-33, wherein the amount of each of the initial and subsequent doses is the same or is different and is independently selected from the group consisting of: about 100 mg, about 200 mg and about 400 mg.
35. The oligonucleotide for use according to any one of embodiments 21-34, wherein the doses are separated in time from each other by at least about four weeks.
36. The oligonucleotide for use according to any one of embodiments 21-35, wherein the doses are separated in time from each other by at least about one month.
37. The oligonucleotide for use according to any one of embodiments 21-36, wherein the doses are separated in time from each other by at least about two months.
38. The oligonucleotide for use according to any one of embodiments 21-37, wherein the doses are separated in time from each other by at least about three months.
39. The oligonucleotide for use according to any one of embodiments 21-38, wherein the doses are separated in time from each other by at least about six months.
40. The oligonucleotide for use according to any one of embodiments 35-39, wherein each of the doses is the same and is selected from an amount of about 1.5 mg/kg, about 3 mg/kg or about 6 mg/kg.
41. The oligonucleotide for use according to any one of embodiments 35-39, wherein each of the doses is the same and is selected from an amount of about 100 mg, about 200 mg or about 400 mg.
42. The oligonucleotide for use according to any one of embodiments 21-34, wherein the doses are separated in time from each other by about four weeks and are administered over a period of about 48 weeks.
43. The oligonucleotide for use according to any one of embodiments 21-34, wherein the doses are separated in time from each other by about one month and are administered over a period of about 48 weeks.
44. The oligonucleotide for use according to any one of embodiments 21-34, wherein the doses are separated in time from each other by about two months and are administered over a period of about 48 weeks.
45. The oligonucleotide for use according to any one of embodiments 21-34, wherein the doses are separated in time from each other by about three months and are administered over a period of about 48 weeks.
46. The oligonucleotide for use according to any one of embodiments 21-34, wherein the doses are separated in time from each other by about four weeks and are administered over a period of about 24 weeks.
47. The oligonucleotide for use according to any one of embodiments 21-34, wherein the doses are separated in time from each other by about one month and are administered over a period of about 24 weeks.
48. The oligonucleotide for use according to any one of embodiments 21-34, wherein the doses are separated in time from each other by about two months and are administered over a period of about 24 weeks.
49. The oligonucleotide for use according to any one of embodiments 21-34, wherein the doses are separated in time from each other by about three months and are administered over a period of about 24 weeks.
50. The oligonucleotide for use according to any one of embodiments 21-34, wherein the doses are separated in time from each other by about four weeks and are administered over a period of about three months.
51. The oligonucleotide for use according to any one of embodiments 21-34, wherein the doses are separated in time from each other by about one month and are administered over a period of about three months.
52. The oligonucleotide for use according to any one of embodiments 21-34, wherein the doses are separated in time from each other by about four weeks and are administered over a period of about 12 weeks.
53. The oligonucleotide for use according to any one of embodiments 21-34, wherein the doses are separated in time from each other by about one month and are administered over a period of about 12 weeks.
54. The oligonucleotide for use according to any one of embodiments 42-53, wherein each of the doses is the same and is selected from an amount of about 1.5 mg/kg, about 3 mg/kg or about 6 mg/kg.
55. The oligonucleotide for use according to any one of embodiments 42-53, wherein each of the doses is the same and is selected from an amount of about 100 mg, about 200 mg or about 400 mg.
56. The oligonucleotide for use according to any one of embodiments 21-34, wherein the doses are each separated in time, each by a period of from about four weeks to about one month.
57. The oligonucleotide for use according to any one of embodiments 21-34, wherein the doses are each separated in time, each by a period of from about four weeks to about two months, for example from about one month to about two months.
58. The oligonucleotide for use according to any one of embodiments 21-34, wherein the doses are each separated in time, each by a period of from about four weeks to about three months, for example from about one month to about three months, for example from about two months to about three months.
59. The oligonucleotide for use according to any one of embodiments 21-34, wherein the doses are each separated in time, each by a period of from about four weeks to about six months, for example from about one month to about six months, for example from about two months to about six months, for example from about three months to about six months.
60. The oligonucleotide for use according to any one of embodiments 21-34, wherein the period of time between each of the doses is independently selected from the group consisting of: about four weeks, about one month, about two months, about three months or about six months.
61. The oligonucleotide for use according to any one of embodiments 21-34, wherein the period of time between each of the doses is as shown in any one of the regimens in Table 1.
62. The oligonucleotide for use according to any one of embodiments 56-61, wherein each of the doses is the same and is selected from an amount of about 1.5 mg/kg, about 3 mg/kg or about 6 mg/kg.
63. The oligonucleotide for use according to any one of embodiments 56-61, wherein each of the doses is the same and is selected from an amount of about 100 mg, about 200 mg or about 400 mg.
64. The oligonucleotide for use according to any one of embodiments 21-63, comprising administering to the patient at least one, at least two, at least three or at least four subsequent doses.
65. The oligonucleotide for use according to any one of embodiments 1-17 or 21-64, wherein the method comprises a treatment holiday, preferably of about three to about six months.
66. The oligonucleotide for use according to any one of embodiments 21-65, wherein the period of time between the initial dose and each of between one and ten, preferably three, subsequent doses is at least about four weeks, the method further comprising a treatment holiday of about three to about six months, after which administration of the oligonucleotide is recommenced.
67. The oligonucleotide for use according to embodiment 66, wherein the recommenced administration comprises between one and ten, preferably three, subsequent doses, preferably wherein each recommenced subsequent dose is separated by a period of time of at least about four weeks.
68. The oligonucleotide for use according to any one of embodiments 1-67, wherein the patient is treatment naïve.
69. The oligonucleotide for use according to any one of embodiments 1-67, wherein the patient is antiviral treatment naïve or the patient has not previously been treated with an antiviral therapy.
70. The oligonucleotide for use according to embodiment 69, wherein the antiviral therapy is an anti-HBV therapy.
71. The oligonucleotide for use according to embodiment 68 or embodiment 69, wherein the patient has not previously been treated with an antiviral therapy for a period of at least about six months.
72. The oligonucleotide for use according to any one of embodiments 69-71, wherein the antiviral therapy is a nucleot(s)ide analogue (NUC), or an interferon-containing agent.
73. The oligonucleotide for use according to any one of embodiments 1-67, wherein the patient is nucleot(s)ide analogue (NUC) suppressed.
74. The oligonucleotide for use according to any one of embodiments 1-67, wherein the patient is immune active.
75. The oligonucleotide for use according to any one of embodiments 1-67, wherein the patient is cirrhotic.
76. The oligonucleotide for use according to any one of embodiments 1-67, wherein the patient is immuno-tolerant.
77. The oligonucleotide for use according to any one of embodiments 1-67, wherein the patient is an inactive carrier.
78. The oligonucleotide for use according to any one of embodiments 68-76, wherein the patient is HBeAg positive.
79. The oligonucleotide for use according to any one of embodiments 68-75 or 77, wherein the patient is HBeAg negative.
80. The oligonucleotide for use according to any one of embodiments 1-67, wherein the patient is HBV delta co-infection.
81. The oligonucleotide for use according to any one of embodiments 68-71, wherein the antiviral therapy is one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; and a CpAM.
82. The oligonucleotide for use according to any one of embodiments 68-72 or 81, wherein the antiviral therapy is entecavir or pro-drug thereof or active thereof, tenofovir or pro-drug thereof or active thereof.
83. The oligonucleotide for use according to any one of embodiments 1-82, wherein the oligonucleotide is administered as a monotherapy.
84. The oligonucleotide for use according to any one of embodiments 1-82, wherein the method further comprises administering an effective amount of at least one additional therapeutic agent.
85. The oligonucleotide for use according to embodiment 84, wherein the additional therapeutic agent is an antiviral agent.
86. The oligonucleotide for use according to embodiment 85, wherein the antiviral agent is an additional anti-HBV agent.
87. The oligonucleotide for use according to embodiment 85, wherein the antiviral agent is one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; and a CpAM.
88. The oligonucleotide for use according to embodiment 85, wherein the antiviral agent is a nucleot(s)ide analogue (NUC).
89. The oligonucleotide for use according to embodiment 88, wherein the antiviral agent is entecavir or pro-drug thereof or active thereof, tenofovir or pro-drug thereof or active thereof.
90. The oligonucleotide for use according to any one of embodiments 84-89, wherein the additional therapeutic agent is administered according to the same or different dosing regimen as the oligonucleotide.
91. The oligonucleotide for use according to any one of embodiments 84-90, wherein the oligonucleotide and the additional therapeutic agent are administered together in a single formulation, or separately in different formulations.
92. The oligonucleotide for use according to any one of embodiments 84-91, wherein the oligonucleotide and the additional therapeutic agent are administered concomitantly.
93. The oligonucleotide for use according to any one of embodiments 84-91, wherein the oligonucleotide and the additional therapeutic agent are administered sequentially.
94. The oligonucleotide for use according embodiment 93, wherein the period of time between the administration of the oligonucleotide and the additional therapeutic agent is about four weeks, about one month, about two months, about 12 weeks, about three months, about 24 weeks or about 6 months, preferably about 12 weeks.
95. The oligonucleotide for use according to any one of embodiments 84-91, 93 or 94, wherein the method comprises a monotherapy lead-in phase, wherein one or more doses of the oligonucleotide are administered prior to the first dose of any additional therapeutic agent.
96. The oligonucleotide for use according to any one of embodiments 84-91, 93 or 94, wherein the method comprises a monotherapy lead-in phase, wherein one or more doses of the oligonucleotide or the pharmaceutical composition are administered prior to a second dose of the additional therapeutic agent.
97. The oligonucleotide for use according to embodiment 84-92, wherein, when administered on the same day, the oligonucleotide and the additional therapeutic agent are administered simultaneously at least once.
98. The oligonucleotide for use according to any one of embodiments 84-91 or 93-96, wherein, when administered on the same day, the oligonucleotide and the additional therapeutic agent are administered sequentially at least once.
99. The oligonucleotide for use according to any one of embodiments 84-92, wherein the oligonucleotide and the additional therapeutic agent are administered together in a single combination formulation.
100. The oligonucleotide for use according to any one of embodiments 84-99, wherein the oligonucleotide and the additional therapeutic agent are administered separately in different formulations.
101. The oligonucleotide for use according to any one of embodiments 1-9, 15-17 and 21-27, 35-40, 42-54, 56-62, 64-100, wherein the patient has not previously been treated with an antiviral therapy, wherein the patient is administered an initial dose of the oligonucleotide of about 1.5 mg/kg followed by three subsequent doses of the oligonucleotide of about 1.5 mg/kg, wherein the doses are separated in time from each other by a period of about four weeks.
102. The oligonucleotide for use according to any one of embodiments 1, 3-9, 15-17 and 21-27, 35-40, 42-54, 56-62, 64-100, wherein the patient has not previously been treated with an antiviral therapy, wherein the patient is administered an initial dose of the oligonucleotide of about 3 mg/kg followed by three subsequent doses of the oligonucleotide of about 3 mg/kg, wherein the doses are separated in time from each other by a period of about four weeks.
103. The oligonucleotide for use according to any one of embodiments 1, 3-9, 15-17 and 21-27, 35-40, 42-54, 56-62, 64-100, wherein the patient has not previously been treated with an antiviral therapy, wherein the patient is administered an initial dose of the oligonucleotide of about 6 mg/kg followed by three subsequent doses of the oligonucleotide of about 6 mg/kg, wherein the doses are separated in time from each other by a period of about four weeks.
104. The oligonucleotide for use according to any one of embodiments 1, 3-9, 15-17 and 21-27, 35-40, 42-54, 56-62, 64-100, wherein the patient has not previously been treated with an antiviral therapy, wherein the method consists of the administration of one dose of the oligonucleotide in an amount of about 1.5 mg/kg.
105. The oligonucleotide for use according to any one of embodiments 1, 3-9, 15-17 and 21-27, 35-40, 42-54, 56-62, 64-100, wherein the patient has not previously been treated with an antiviral therapy, wherein the method consists of the administration of one dose of the oligonucleotide in an amount of about 3 mg/kg.
106. The oligonucleotide for use according to any one of embodiments 1, 3-9, 15-17 and 21-27, 35-40, 42-54, 56-62, 64-100, wherein the patient has not previously been treated with an antiviral therapy, wherein the method consists of the administration of one dose of the oligonucleotide in an amount of about 6 mg/kg.
107. The oligonucleotide for use according to any one of embodiments 1, 3-9, 15-17 and 21-27, 35-40, 42-54, 56-62, 64-100, wherein the patient has not previously been treated with an antiviral therapy, wherein the method comprises the administration of a single dose of the oligonucleotide in an amount of about 1.5 mg/kg.
108. The oligonucleotide for use according to any one of embodiments 1, 3-9, 15-17 and 21-27, 35-40, 42-54, 56-62, 64-100, wherein the patient has not previously been treated with an antiviral therapy, wherein the method comprises the administration of a single dose of the oligonucleotide in an amount of about 3 mg/kg.
109. The oligonucleotide for use according to any one of embodiments 1, 3-9, 15-17 and 21-27, 35-40, 42-54, 56-62, 64-100, wherein the patient has not previously been treated with an antiviral therapy, wherein the method comprises the administration of a single dose of the oligonucleotide in an amount of about 6 mg/kg.
110. The oligonucleotide for use according to any one of embodiments 2-4, 10-17 and 28-39, 41-53, 55-61, 63-100, wherein the patient has not previously been treated with an antiviral therapy, wherein the patient is administered an initial dose of the oligonucleotide of 90 or 100 mg followed by three subsequent doses of the oligonucleotide of 90 or 100 mg, wherein the doses are separated in time from each other by a period of about four weeks.
111. The oligonucleotide for use according to any one of embodiments 2-4, 10-17 and 28-39, 41-53, 55-61, 63-100, wherein the patient has not previously been treated with an antiviral therapy, wherein the patient is administered an initial dose of the oligonucleotide of 200 or 210 mg followed by three subsequent doses of the oligonucleotide of 200 or 210 mg, wherein the doses are separated in time from each other by a period of about four weeks.
112. The oligonucleotide for use according to any one of embodiments 2-4, 10-17 and 28-39, 41-53, 55-61, 63-100, wherein the patient has not previously been treated with an antiviral therapy, wherein the patient is administered an initial dose of the oligonucleotide of 360 or 400 mg followed by three subsequent doses of the oligonucleotide of 360 or 400 mg, wherein the doses are separated in time from each other by a period of about four weeks.
113. The oligonucleotide for use according to any one of embodiments 2-4, 10-17 and 28-39, 41-53, 55-61, 63-100, wherein the patient has not previously been treated with an antiviral therapy, wherein the method consists of the administration of one dose of the oligonucleotide in an amount of about 90 or 100 mg.
114. The oligonucleotide for use according to any one of embodiments 2-4, 10-17 and 28-39, 41-53, 55-61, 63-100, wherein the patient has not previously been treated with an antiviral therapy, wherein the method consists of the administration of one dose of the oligonucleotide in an amount of about 200 or 210 mg.
115. The oligonucleotide for use according to any one of embodiments 2-4, 10-17 and 28-39, 41-53, 55-61, 63-100, wherein the patient has not previously been treated with an antiviral therapy, wherein the method consists of the administration of one dose of the oligonucleotide in an amount of about 360 or 400 mg.
116. The oligonucleotide for use according to any one of embodiments 2-4, 10-17 and 28-39, 41-53, 55-61, 63-100, wherein the patient has not previously been treated with an antiviral therapy, wherein the method comprises the administration of a single dose of the oligonucleotide in an amount of about 90 or 100 mg.
117. The oligonucleotide for use according to any one of embodiments 2-4, 10-17 and 28-39, 41-53, 55-61, 63-100, wherein the patient has not previously been treated with an antiviral therapy, wherein the method comprises the administration of a single dose of the oligonucleotide in an amount of about 200 or 210 mg.
118. The oligonucleotide for use according to any one of embodiments 2-4, 10-17 and 28-39, 41-53, 55-61, 63-100, wherein the patient has not previously been treated with an antiviral therapy, wherein the method comprises the administration of a single dose of the oligonucleotide in an amount of about 360 or 400 mg.
119. The oligonucleotide for use according to any one of embodiments 101-118, wherein the method further comprises the administration of an antiviral agent, preferably a nucleot(s)ide analogue (NUC), wherein the antiviral agent is administered sequentially to the oligonucleotide, preferably wherein the period of time between the administration of the oligonucleotide and the antiviral agent is about 12 weeks.
120. The oligonucleotide for use according to any one of embodiments 1-119, wherein the hepatitis B virus is selected from any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).
121. The oligonucleotide for use according to any one of embodiments 1-120, wherein the oligonucleotide comprises a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and one phosphorothioate linkage between the nucleotides at positions 1 and 2,
      wherein each of the nucleotides of the -GAAA- sequence on the sense strand is conjugated to a monovalent GalNAc moiety; wherein the -GAAA- sequence comprises the structure:

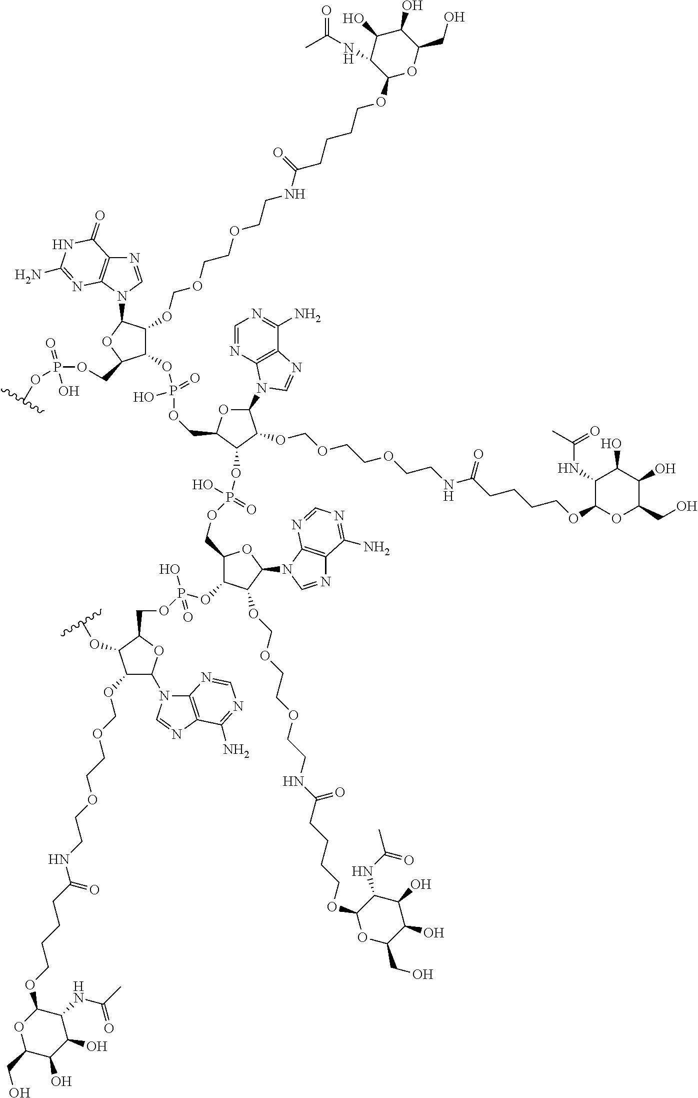 [SMILES: *O[C@@H]1[C@@H](COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(=O)[nH]c(N)nc43)O[C@@H]2COP(=O)(O)O*)OC(n2cnc3c(N)ncnc32)[C@@H]1OCOCCOCCNC(=O)CCCCO[C@@H]1O[C@H](CO)[C@H](O)[C@H](O)[C@H]1NC(C)=O]

and
the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and five phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 5′-nucleotide of the antisense strand has the following structure:

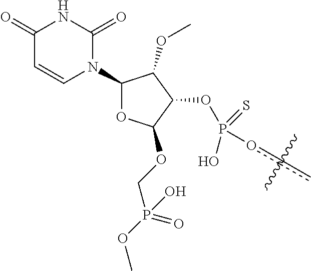

or a pharmaceutically acceptable salt thereof.
122. The oligonucleotide for use according to any one of embodiments 1-121, wherein the oligonucleotide is in the form of a pharmaceutically acceptable salt.
123. The oligonucleotide for use according to embodiment 122, wherein the pharmaceutically acceptable salt is a sodium salt.
124. The oligonucleotide for use according to embodiment 122, wherein the pharmaceutically acceptable salt is a potassium salt.
125. The oligonucleotide for use according to embodiment 122, wherein the pharmaceutically acceptable salt of the oligonucleotide is as shown in FIG. 2A or FIG. 2B.
126. A pharmaceutical composition comprising the oligonucleotide or pharmaceutically acceptable salt thereof of any one of embodiment 1-125 and a pharmaceutically acceptable solvent, carrier, excipient, diluent or adjuvant for use according to any one of embodiment 1-125.
127. The pharmaceutical composition for use according to embodiment 126, wherein the pharmaceutically acceptable solvent, carrier, excipient, diluent or adjuvant comprises saline.
128. The pharmaceutical composition for use according to embodiment 127, wherein the saline is phosphate buffered saline.
129. The pharmaceutical composition for use according to embodiment 126, wherein the pharmaceutically acceptable solvent, carrier, excipient, diluent or adjuvant is water, for example water for injection.
130. The oligonucleotide for use according to any one of embodiments 96-99, wherein the administration of the oligonucleotide provides clinical benefit as measured by one or more of the following:
   (a) reduction in HBsAg level, preferably at least 1 log reduction;
   (b) at least 1 log reduction in HBsAg level as determined at least 50 days following the initial dose;
   (c) reduction in HBV DNA level, preferably at least 2 log reduction;
   (d) at least 2 log reduction in HBV DNA level as determined at least 25 days following the initial dose;
   (e) reduction in HBV DNA level by 90%;
   (f) reduction in HBcrAg level, preferably at least 1 log reduction;
   (g) at least 1 log reduction in HBcrAg level as determined at least 25 days following the initial dose;
   (h) reduction in HBeAg level, preferably at least 1 log reduction;
   (i) at least 1 log reduction in HBcrAg level as determined at least 50 days following the initial dose;
   (j) the presence of HBV antigen is sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems;
   (k) the presence of HBV antigen is sufficiently reduced to result in PHBV, defined as serum HBeAg absence plus serum HBeAb presence if monitoring HBeAg as the determinant for seroconversion, or defined as serum HBsAg absence if monitoring HBsAg as the determinant for PHBV, as determined by currently available detection limits of commercial ELISA systems;
   (l) at least three-fold increase in ALT level;
   (m) at least three-fold increase in ALT level as determined at between about 20 and about 70 days following the initial dose;
   (n) induction of a host-mediated, cell-mediated immune response, such as a T-cell response;
   (o) substantially no significant change in the level of albumin and bilirubin, as determined at any point following the initial dose;
   (p) lack of patient rebound.
131. The oligonucleotide for use according to any one of embodiments 1-130, wherein the method further comprises the step of determining the level of a biomarker, for example HBsAg, HBeAg or HbcrAg, in a sample obtained from the patient.
132. The oligonucleotide for use according to embodiment 131, wherein the determining step is carried out during a treatment holiday.
133. The oligonucleotide for use according to embodiment 131 or 132, comprising the step of administering one or more further doses of the oligonucleotide following the determining step.
134. A method for treating hepatitis B or hepatitis B virus (HBV) infection in a human patient, the method comprising administering to the patient an oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
      wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP),
      or a pharmaceutically acceptable salt thereof;
   the method comprising administering to the patient via subcutaneous route an initial dose of from about 0.1 mg/kg to about 12 mg/kg of the oligonucleotide.
135. A method for treating hepatitis B or hepatitis B virus (HBV) infection in a human patient, the method comprising administering to the patient an oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
      wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP),
      or a pharmaceutically acceptable salt thereof;
   the method comprising administering to the patient via subcutaneous route an initial dose of from about 6 mg to about 800 mg of the oligonucleotide.
136. The method according to embodiment 134 or embodiment 135, wherein the hepatitis B or HBV infection is chronic hepatitis B or chronic HBV infection.
137. The method according to any one of embodiments 134-136, wherein the oligonucleotide is administered by subcutaneous injection.
138. The method according to any one of embodiments 134 or 136-137, wherein the initial dose is from about 0.5 mg/kg to about 10 mg/kg.
139. The method according to any one of embodiments 134 or 136-138, wherein the initial dose is from about 1.5 mg/kg to about 6 mg/kg.
140. The method according to any one of embodiments 134 or 136-139, wherein the initial dose is about 1.5 mg/kg.
141. The method according to any one of embodiments 134 or 136-139, wherein the initial dose is about 3 mg/kg.
142. The method according to any one of embodiments 134 or 136-139, wherein the initial dose is about 6 mg/kg.
143. The method according to any one of embodiments 135-137, wherein the initial dose is from about 34 mg to about 667 mg.
144. The method according to any one of embodiments 135-137 or 143, wherein the initial dose is from about 100 mg to about 400 mg.
145. The method according to any one of embodiments 135-137 or 143-144, wherein the initial dose is about 100 mg.
146. The method according to any one of embodiments 135-137 or 143-144, wherein the initial dose is about 200 mg.
147. The method according to any one of embodiments 135-137 or 143-144, wherein the initial dose is about 400 mg.
148. The method according to any one of embodiments 134-147, wherein the initial dose is a single dose or is the only dose administered.
149. The method according to any one of embodiments 134-148, wherein the method consists of administering the oligonucleotide.
150. The method according to any one of embodiments 134-149, wherein the method comprises or consists of the administering the oligonucleotide, to the exclusion of other anti-HBV agents.
151. The method according to any one of embodiments 134-150, wherein the method comprises administering a single dose of the oligonucleotide.
152. The method according to any one of embodiments 134-151, wherein the method consists of administering a single dose of the oligonucleotide.
153. The method according to any one of embodiments 134-152, wherein the treatment of hepatitis B or HBV infection is provided by said administering of said initial dose, said one dose or said single dose of the oligonucleotide.
154. The method according to any one of embodiments 134, 136-142 or 148-150, further comprising administering to the patient one or more subsequent doses of the oligonucleotide in an amount that is from about 0.1 mg/kg to about 12 mg/kg.
155. The method according to embodiment 154, wherein the subsequent dose(s) is from about 0.5 mg/kg to about 10 mg/kg.
156. The method according to embodiment 154 or embodiment 155, wherein the subsequent dose(s) is from about 1.5 mg/kg to about 6 mg/kg.
157. The method according to any one of embodiments 154-156, wherein the subsequent dose(s) is about 1.5 mg/kg.
158. The method according to any one of embodiments 154-156, wherein subsequent dose(s) is about 3 mg/kg.
159. The method according to any one of embodiments 154-156, wherein subsequent dose(s) is about 6 mg/kg.
160. The method according to any one of embodiments 154-159, wherein the amount of each of the initial and subsequent doses is the same or is different and is independently selected from the group consisting of: about 1.5 mg/kg, about 3 mg/kg and about 6 mg/kg.
161. The method according to any one of embodiments 135-137 or 143-150, further comprising administering to the patient one or more subsequent doses of the oligonucleotide in an amount that is from about 6 mg to about 800 mg.
162. The method according to embodiment 161, wherein the subsequent dose(s) is from about 34 mg to about 667 mg.
163. The method according to embodiment 161 or embodiment 162, wherein the subsequent dose(s) is from about 100 mg to about 400 mg.
164. The method according to any one of embodiments 161-163, wherein the subsequent dose(s) is about 100 mg.
165. The method according to any one of embodiments 161-163, wherein subsequent dose(s) is about 200 mg.
166. The method according to any one of embodiments 161-163, wherein subsequent dose(s) is about 400 mg.
167. The method according to any one of embodiments 161-166, wherein the amount of each of the initial and subsequent doses is the same or is different and is independently selected from the group consisting of: about 100 mg, about 200 mg and about 400 mg.
168. The method according to any one of embodiments 154-167, wherein the doses are separated in time from each other by at least about four weeks.
169. The method according to any one of embodiments 154-168, wherein the doses are separated in time from each other by at least about one month.
170. The method according to any one of embodiments 154-169, wherein the doses are separated in time from each other by at least about two months.
171. The method according to any one of embodiments 154-170, wherein the doses are separated in time from each other by at least about three months.
172. The method according to any one of embodiments 154-171, wherein the doses are separated in time from each other by at least about six months.
173. The method according to any one of embodiments 168-172, wherein each of the doses is the same and is selected from an amount of about 1.5 mg/kg, about 3 mg/kg or about 6 mg/kg.
174. The method according to any one of embodiments 168-172, wherein each of the doses is the same and is selected from an amount of about 100 mg, about 200 mg or about 400 mg.
175. The method according to any one of embodiments 154-167, wherein the doses are separated in time from each other by about four weeks and are administered over a period of about 48 weeks.
176. The method according to any one of embodiments 154-167, wherein the doses are separated in time from each other by about one month and are administered over a period of about 48 weeks.
177. The method according to any one of embodiments 154-167, wherein the doses are separated in time from each other by about two months and are administered over a period of about 48 weeks.
178. The method according to any one of embodiments 154-167, wherein the doses are separated in time from each other by about three months and are administered over a period of about 48 weeks.
179. The method according to any one of embodiments 154-167, wherein the doses are separated in time from each other by about four weeks and are administered over a period of about 24 weeks.
180. The method according to any one of embodiments 154-167, wherein the doses are separated in time from each other by about one month and are administered over a period of about 24 weeks.
181. The method according to any one of embodiments 154-167, wherein the doses are separated in time from each other by about two months and are administered over a period of about 24 weeks.
182. The method according to any one of embodiments 154-167, wherein the doses are separated in time from each other by about three months and are administered over a period of about 24 weeks.
183. The method according to any one of embodiments 154-167, wherein the doses are separated in time from each other by about four weeks and are administered over a period of about three months.
184. The method according to any one of embodiments 154-167, wherein the doses are separated in time from each other by about one month and are administered over a period of about three months.
185. The method according to any one of embodiments 154-167, wherein the doses are separated in time from each other by about four weeks and are administered over a period of about 12 weeks.
186. The method according to any one of embodiments 154-167, wherein the doses are separated in time from each other by about one month and are administered over a period of about 12 weeks.
187. The method according to any one of embodiments 175-186, wherein each of the doses is the same and is selected from an amount of about 1.5 mg/kg, about 3 mg/kg or about 6 mg/kg.
188. The method according to any one of embodiments 175-186, wherein each of the doses is the same and is selected from an amount of about 100 mg, about 200 mg or about 400 mg.
189. The method according to any one of embodiments 154-167, wherein the doses are each separated in time, each by a period of from about four weeks to about one month.
190. The method according to any one of embodiments 154-167, wherein the doses are each separated in time, each by a period of from about four weeks to about two months, for example from about one month to about two months.
191. The method according to any one of embodiments 154-167, wherein the doses are each separated in time, each by a period of from about four weeks to about three months, for example from about one month to about three months, for example from about two months to about three months.
192. The method according to any one of embodiments 154-167, wherein the doses are each separated in time, each by a period of from about four weeks to about six months, for example from about one month to about six months, for example from about two months to about six months, for example from about three months to about six months.
193. The method according to any one of embodiments 154-167, wherein the period of time between each of the doses is independently selected from the group consisting of: about four weeks, about one month, about two months, about three months or about six months.
194. The method according to any one of embodiments 154-167, wherein the period of time between each of the doses is as shown in any one of the regimens in Table 1.
195. The method according to any one of embodiments 189-194, wherein each of the doses is the same and is selected from an amount of about 1.5 mg/kg, about 3 mg/kg or about 6 mg/kg.
196. The method according to any one of embodiments 189-194, wherein each of the doses is the same and is selected from an amount of about 100 mg, about 200 mg or about 400 mg.
197. The method according to any one of embodiments 154-196, comprising administering to the patient at least one, at least two, at least three or at least four subsequent doses.
198. The method according to any one of embodiments 134-150 or 154-197, wherein the method comprises a treatment holiday, preferably of about three to about six months.
199. The method according to any one of embodiments 154-198, wherein the period of time between the initial dose and each of between one and ten, preferably three, subsequent doses is at least about four weeks, the method further comprising a treatment holiday of about three to about six months, after which administration of the oligonucleotide is recommenced.
200. The method according to embodiment 199, wherein the recommenced administration comprises between one and ten, preferably three, subsequent doses, preferably wherein each recommenced subsequent dose is separated by a period of time of at least about four weeks.
201. The method according to any one of embodiments 134-200, wherein the patient is treatment naïve.
202. The method according to any one of embodiments 134-200, wherein the patient is antiviral treatment naïve or the patient has not previously been treated with an antiviral therapy.
203. The method according to embodiment 202, wherein the antiviral therapy is an anti-HBV therapy.
204. The method according to embodiment 201 or embodiment 202, wherein the patient has not previously been treated with an antiviral therapy for a period of at least about six months.
205. The method according to any one of embodiments 202-204, wherein the antiviral therapy is a nucleot(s)ide analogue (NUC), or an interferon-containing agent.
206. The method according to any one of embodiments 134-200, wherein the patient is nucleot(s)ide analogue (NUC) suppressed.
207. The method according to any one of embodiments 134-200, wherein the patient is immune active.
208. The method according to any one of embodiments 134-200, wherein the patient is cirrhotic.
209. The method according to any one of embodiments 134-200, wherein the patient is immuno-tolerant.
210. The method according to any one of embodiments 134-200, wherein the patient is an inactive carrier.
211. The method according to any one of embodiments 201-209, wherein the patient is HBeAg positive.
212. The method according to any one of embodiments 201-208 or 210, wherein the patient is HBeAg negative.
213. The method according to any one of embodiments 134-200, wherein the patient is HBV delta co-infection.
214. The method according to any one of embodiments 201-204, wherein the antiviral therapy is one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; and a CpAM.
215. The method according to any one of embodiments 201-205 or 14, wherein the antiviral therapy is entecavir or pro-drug thereof or active thereof, tenofovir or pro-drug thereof or active thereof.
216. The method according to any one of embodiments 134-215, wherein the oligonucleotide is administered as a monotherapy.
217. The method according to any one of embodiments 134-215, wherein the method further comprises administering an effective amount of at least one additional therapeutic agent.
218. The method according to embodiment 217, wherein the additional therapeutic agent is an antiviral agent.
219. The method according to embodiment 218, wherein the antiviral agent is an additional anti-HBV agent.
220. The method according to embodiment 218, wherein the antiviral agent is one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; and a CpAM.
221. The method according to embodiment 218, wherein the antiviral agent is a nucleot(s)ide analogue (NUC).
222. The method according to embodiment 221, wherein the antiviral agent is entecavir or pro-drug thereof or active thereof, tenofovir or pro-drug thereof or active thereof.
223. The method according to any one of embodiments 217-222, wherein the additional therapeutic agent is administered according to the same or different dosing regimen as the oligonucleotide.
224. The method according to any one of embodiments 217-223, wherein the oligonucleotide and the additional therapeutic agent are administered together in a single formulation, or separately in different formulations.
225. The method according to any one of embodiments 217-224, wherein the oligonucleotide and the additional therapeutic agent are administered concomitantly.
226. The method according to any one of embodiments 217-224, wherein the oligonucleotide and the additional therapeutic agent are administered sequentially.
227. The method according embodiment 226, wherein the period of time between the administration of the oligonucleotide and the additional therapeutic agent is about four weeks, about one month, about two months, about 12 weeks, about three months, about 24 weeks or about 6 months, preferably about 12 weeks.
228. The method according to any one of embodiments 217-224, 226 or 227, wherein the method comprises a monotherapy lead-in phase, wherein one or more doses of the oligonucleotide are administered prior to the first dose of any additional therapeutic agent.
229. The method according to any one of embodiments 217-224, 226 or 227, wherein the method comprises a monotherapy lead-in phase, wherein one or more doses of the oligonucleotide or the pharmaceutical composition are administered prior to a second dose of the additional therapeutic agent.
230. The method according to embodiment 217-225, wherein, when administered on the same day, the oligonucleotide and the additional therapeutic agent are administered simultaneously at least once.
231. The method according to any one of embodiments 217-224 or 226-229, wherein, when administered on the same day, the oligonucleotide and the additional therapeutic agent are administered sequentially at least once.
232. The method according to any one of embodiments 217-225, wherein the oligonucleotide and the additional therapeutic agent are administered together in a single combination formulation.
233. The method according to any one of embodiments 217-232, wherein the oligonucleotide and the additional therapeutic agent are administered separately in different formulations.
234. The method according to any one of embodiments 134-142, 148-150, 154-160, 168-173, 175-187, 189-195, 197-233, wherein the patient has not previously been treated with an antiviral therapy, wherein the patient is administered an initial dose of the oligonucleotide of 1.5 mg/kg followed by three subsequent doses of the oligonucleotide of 1.5 mg/kg, wherein the doses are separated in time from each other by a period of about four weeks.
235. The method according to any one of embodiments 134, 136-142, 148-150, 154-160, 168-173, 175-187, 189-195, 197-233, wherein the patient has not previously been treated with an antiviral therapy, wherein the patient is administered an initial dose of the oligonucleotide of 3 mg/kg followed by three subsequent doses of the oligonucleotide of 3 mg/kg, wherein the doses are separated in time from each other by a period of about four weeks.
236. The method according to any one of embodiments 134, 136-142, 148-150, 154-160, 168-173, 175-187, 189-195, 197-233, wherein the patient has not previously been treated with an antiviral therapy, wherein the patient is administered an initial dose of the oligonucleotide of 6 mg/kg followed by three subsequent doses of the oligonucleotide of 6 mg/kg, wherein the doses are separated in time from each other by a period of about four weeks.
237. The method according to any one of embodiments 134, 136-142, 148-150, 154-160, 168-173, 175-187, 189-195, 197-233, wherein the patient has not previously been treated with an antiviral therapy, wherein the method consists of the administration of one dose of the oligonucleotide in an amount of about 1.5 mg/kg.
238. The method according to any one of embodiments 134, 136-142, 148-150, 154-160, 168-173, 175-187, 189-195, 197-233, wherein the patient has not previously been treated with an antiviral therapy, wherein the method consists of the administration of one dose of the oligonucleotide in an amount of about 3 mg/kg.
239. The method according to any one of embodiments 134, 136-142, 148-150, 154-160, 168-173, 175-187, 189-195, 197-233, wherein the patient has not previously been treated with an antiviral therapy, wherein the method consists of the administration of one dose of the oligonucleotide in an amount of about 6 mg/kg.
240. The method according to any one of embodiments 134, 136-142, 148-150, 154-160, 168-173, 175-187, 189-195, 197-233, wherein the patient has not previously been treated with an antiviral therapy, wherein the method comprises the administration of a single dose of the oligonucleotide in an amount of about 1.5 mg/kg.
241. The method according to any one of embodiments 134, 136-142, 148-150, 154-160, 168-173, 175-187, 189-195, 197-233, wherein the patient has not previously been treated with an antiviral therapy, wherein the method comprises the administration of a single dose of the oligonucleotide in an amount of about 3 mg/kg.
242. The method according to any one of embodiments 134, 136-142, 148-150, 154-160, 168-173, 175-187, 189-195, 197-233, wherein the patient has not previously been treated with an antiviral therapy, wherein the method comprises the administration of a single dose of the oligonucleotide in an amount of about 6 mg/kg.
243. The method according to any one of embodiments 135-137, 143-150, 161-172, 174-186, 188-194, 196-233, wherein the patient has not previously been treated with an antiviral therapy, wherein the patient is administered an initial dose of the oligonucleotide of 90 or 100 mg followed by three subsequent doses of the oligonucleotide of 90 or 100 mg, wherein the doses are separated in time from each other by a period of about four weeks.
244. The method according to any one of embodiments 135-137, 143-150, 161-172, 174-186, 188-194, 196-233, wherein the patient has not previously been treated with an antiviral therapy, wherein the patient is administered an initial dose of the oligonucleotide of 200 or 210 mg followed by three subsequent doses of the oligonucleotide of 200 or 210 mg, wherein the doses are separated in time from each other by a period of about four weeks.
245. The method according to any one of embodiments 135-137, 143-150, 161-172, 174-186, 188-194, 196-233, wherein the patient has not previously been treated with an antiviral therapy, wherein the patient is administered an initial dose of the oligonucleotide of 360 or 400 mg followed by three subsequent doses of the oligonucleotide of 360 or 400 mg, wherein the doses are separated in time from each other by a period of about four weeks.
246. The method according to any one of embodiments 135-137, 143-150, 161-172, 174-186, 188-194, 196-233, wherein the patient has not previously been treated with an antiviral therapy, wherein the method consists of the administration of one dose of the oligonucleotide in an amount of about 90 or 100 mg.
247. The method according to any one of embodiments 135-137, 143-150, 161-172, 174-186, 188-194, 196-233, wherein the patient has not previously been treated with an antiviral therapy, wherein the method consists of the administration of one dose of the oligonucleotide in an amount of about 200 or 210 mg.
248. The method according to any one of embodiments 135-137, 143-150, 161-172, 174-186, 188-194, 196-233, wherein the patient has not previously been treated with an antiviral therapy, wherein the method consists of the administration of one dose of the oligonucleotide in an amount of about 360 or 400 mg.
249. The method according to any one of embodiments 135-137, 143-150, 161-172, 174-186, 188-194, 196-233, wherein the patient has not previously been treated with an antiviral therapy, wherein the method comprises the administration of a single dose of the oligonucleotide in an amount of about 90 or 100 mg.
250. The method according to any one of embodiments 135-137, 143-150, 161-172, 174-186, 188-194, 196-233, wherein the patient has not previously been treated with an antiviral therapy, wherein the method comprises the administration of a single dose of the oligonucleotide in an amount of about 200 or 210 mg.
251. The method according to any one of embodiments 135-137, 143-150, 161-172, 174-186, 188-194, 196-233, wherein the patient has not previously been treated with an antiviral therapy, wherein the method comprises the administration of a single dose of the oligonucleotide in an amount of about 360 or 400 mg.
252. The method according to any one of embodiments 234-251, wherein the method further comprises the administration of an antiviral agent, preferably a nucleot(s)ide analogue (NUC), wherein the antiviral agent is administered sequentially to the oligonucleotide, preferably wherein the period of time between the administration of the oligonucleotide and the antiviral agent is about 12 weeks.
253. The method according to any one of embodiments 134-252, wherein the hepatitis B virus is selected from any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).
254. The method according to any one of embodiments 134-253, wherein the oligonucleotide duplex comprises a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and one phosphorothioate linkage between the nucleotides at positions 1 and 2,
      wherein each of the nucleotides of the -GAAA- sequence on the sense strand is conjugated to a monovalent GalNAc moiety; wherein the -GAAA- sequence comprises the structure:

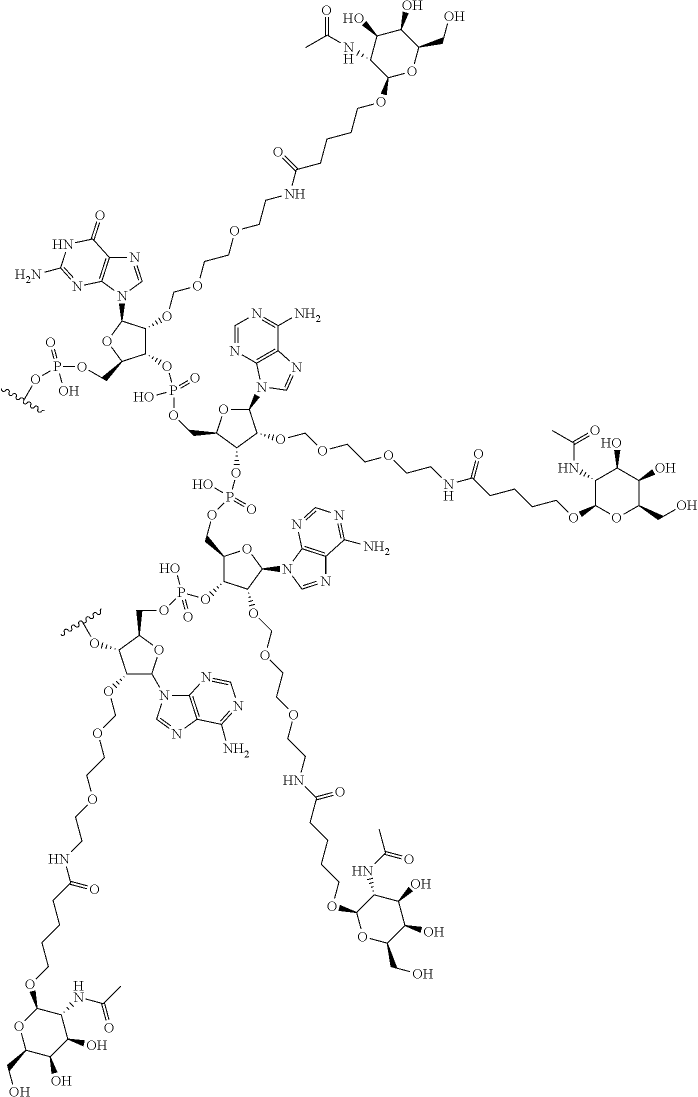 [SMILES: *O[C@@H]1[C@@H](COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(=O)[nH]c(N)nc43)O[C@@H]2COP(=O)(O)O*)OC(n2cnc3c(N)ncnc32)[C@@H]1OCOCCOCCNC(=O)CCCCO[C@@H]1O[C@H](CO)[C@H](O)[C@H](O)[C@H]1NC(C)=O]

the antisense strand consists of a sequence as set forth in UUAUUGUGAGGAUUUUUGUCGG (SEQ ID NO: 2) and comprising
   2′-fluoro modified nucleotides at positions 2, 3, 5, 7, 8, 10, 12, 14, 16, and 19,
   2′-O-methyl modified nucleotides at positions 1, 4, 6, 9, 11, 13, 15, 17, 18, and 20-22, and five phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
   wherein the 5′-nucleotide of the antisense strand has the following structure:

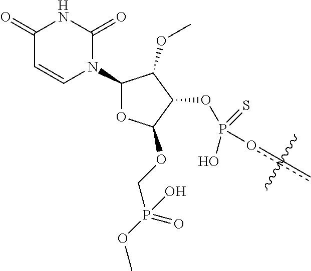

or a pharmaceutically acceptable salt thereof.
255. The method according to any one of embodiments 134-254, wherein the oligonucleotide is in the form of a pharmaceutically acceptable salt.
256. The method according to embodiment 255, wherein the pharmaceutically acceptable salt is a sodium salt.
257. The method according to embodiment 255, wherein the pharmaceutically acceptable salt is a potassium salt.
258. The method according to embodiment 255, wherein the pharmaceutically acceptable salt of the oligonucleotide is as shown in FIG. 2A or FIG. 2B.
259. A method for treating hepatitis B or hepatitis B virus (HBV) infection in a human patient, the method comprising administering to the patient a pharmaceutical composition comprising the oligonucleotide or pharmaceutically acceptable salt thereof of any one of embodiment 1-125 and a pharmaceutically acceptable solvent, carrier, excipient, diluent or adjuvant, the method comprising administering to the patient via subcutaneous route an initial dose of from about 0.1 mg/kg to about 12 mg/kg of the oligonucleotide.
260. The method according to embodiment 259, wherein the pharmaceutically acceptable solvent, carrier, excipient, diluent or adjuvant comprises saline.
261. The method according to embodiment 260, wherein the saline is phosphate buffered saline.
262. The method according to embodiment 259, wherein the pharmaceutically acceptable solvent, carrier, excipient, diluent or adjuvant is water, for example water for injection.
263. The method according to any one of embodiments 229-232, wherein the administration of the oligonucleotide provides clinical benefit as measured by one or more of the following:
   (a) reduction in HBsAg level, preferably at least 1 log reduction;
   (b) at least 1 log reduction in HBsAg level as determined at least 50 days following the initial dose;
   (c) reduction in HBV DNA level, preferably at least 2 log reduction;
   (d) at least 2 log reduction in HBV DNA level as determined at least 25 days following the initial dose;
   (e) reduction in HBV DNA level by 90%;
   (f) reduction in HBcrAg level, preferably at least 1 log reduction;
   (g) at least 1 log reduction in HBcrAg level as determined at least 25 days following the initial dose;
   (h) reduction in HBeAg level, preferably at least 1 log reduction;
   (i) at least 1 log reduction in HBcrAg level as determined at least 50 days following the initial dose;
   (j) the presence of HBV antigen is sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems;
   (k) the presence of HBV antigen is sufficiently reduced to result in PHBV, defined as serum HBeAg absence plus serum HBeAb presence if monitoring HBeAg as the determinant for seroconversion, or defined as serum HBsAg absence if monitoring HBsAg as the determinant for PHBV, as determined by currently available detection limits of commercial ELISA systems;
   (l) at least three-fold increase in ALT level;
   (m) at least three-fold increase in ALT level as determined at between about 20 and about 70 days following the initial dose;
   (n) induction of a host-mediated, cell-mediated immune response, such as a T-cell response;
   (o) substantially no significant change in the level of albumin and bilirubin, as determined at any point following the initial dose;
   (p) lack of patient rebound.
264. The method according to any one of embodiments 134-263, wherein the method further comprises the step of measuring the level of a biomarker, for example HBsAg, HBeAg or HbcrAg, in a sample obtained from the patient.
265. The method according to embodiment 264, wherein the measuring step is carried out during a treatment holiday.
266. The method according to embodiment 264 or 265, comprising the step of administering one or more further doses of the oligonucleotide following the measuring step.
267. Use of an oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
   wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP),
      or a pharmaceutically acceptable salt thereof;
   in the manufacture of a medicament for the treatment hepatitis B or hepatitis B virus (HBV) infection in a human patient
   comprising administering to the patient via the subcutaneous route an initial dose of from about 0.1 mg/kg to about 12 mg/kg of the oligonucleotide.
268. Use of an oligonucleotide comprising a sense strand forming a duplex region with an antisense strand, wherein:
   the sense strand consists of a sequence as set forth in GACAAAAAUCCUCACAAUAAGCAGCCGAAAGGCUGC (SEQ ID NO: 1) and comprising
      2′-fluoro modified nucleotides at positions 3, 8-10, 12, 13, and 17,
      2′-O-methyl modified nucleotides at positions 1, 2, 4-7, 11, 14-16, 18-26, and 31-36, and a phosphorothioate linkage between the nucleotides at positions 1 and 2,
      wherein each of the nucleotides of the -GAAA- sequence on the sense strand is conjugated to a monovalent GalNAc moiety; and
   the antisense strand consists of a sequence as set forth in UUAUUGUGAGGAUUUUUGUCGG (SEQ ID NO: 2) and comprising
      2′-fluoro modified nucleotides at positions 2, 3, 5, 7, 8, 10, 12, 14, 16, and 19,
      2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
      wherein the 4′-carbon of the sugar of the 5′-nucleotide of the antisense strand comprises a methoxy phosphonate (MOP),
      or a pharmaceutically acceptable salt thereof;
   in the manufacture of a medicament for the treatment hepatitis B or hepatitis B virus (HBV) infection in a human patient
   comprising administering to the patient via the subcutaneous route an initial dose of from about 6 mg to about 800 mg of the oligonucleotide.

### VII. Examples

### Example 1: HBVS-219

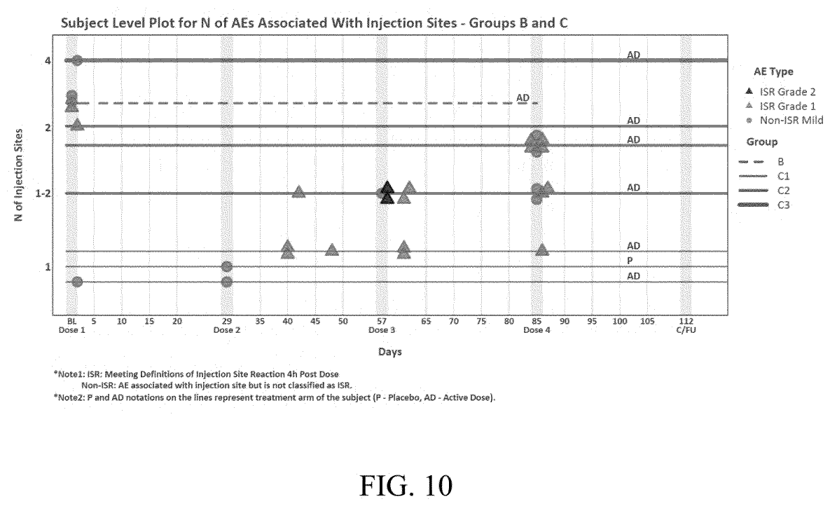

An oligonucleotide according to the invention was identified and produced in WO2019/079781 (incorporated herein by reference in its entirety). FIG. 1 corresponding to FIG. 10 of WO2019/079781, illustrates an example of a modified duplex structure for HBVS-219 with an incorporated mismatch. The mismatch is made relative to HBV genotypes A-J in accordance with WO2019/079781, Example 2 therein. The sense strand spans nucleotides 1 through 36 and the antisense strand spans oligonucleotides 1 through 22, the latter strand shown numbered in right-to-left orientation. The duplex form is shown with a nick between nucleotides at position 36 in the sense strand and position 1 in the antisense strand. Modifications in the sense strand were as follows: 2′-fluoro modified nucleotides at positions 3, 8-10, 12, 13, and 17; 2′-O-methyl modified nucleotides at positions 1, 2, 4-7, 11, 14-16, 18-26, and 31-36; a phosphorothioate internucleotide linkage between nucleotides at positions 1 and 2; 2′-OH nucleotides at positions 27-30; a 2′-aminodiethoxymethanol-Guanidine-GalNAc at position 27; and a 2′-aminodiethoxymethanol-Adenine-GalNAc at each of positions 28, 29, and 30. Modifications in the antisense strand were as follows: 5′-Methoxy, Phosphonate-4′-oxy-2′-O-methyluridine phosphorothioate at position 1; 2′-fluoro modified nucleotides at positions 2, 3, 5, 7, 8, 10, 12, 14, 16, and 19; 2′-O-methyl modified nucleotides at positions 1, 4, 6, 9, 11, 13, 15, 17, 18, and 20-22; and phosphorothioate internucleotide linkages between nucleotides at positions 1 and 2, 2 and 3, 3 and 4, 20 and 21, and 21 and 22. The antisense strand included an incorporated mismatch at position 15. Also as shown, the antisense strand of the duplex included a “GG” overhang spanning positions 21-22.

### Example 2: Evaluation of the Safety, Tolerability in Healthy Human Subjects and Efficacy of HBVS-219 in HBV Patients

This study was designed to evaluate the safety and tolerability in healthy subjects (Group A) and the efficacy of HBVS-219 in HBV patients (Groups B and C). The structure of HBVS-219 is shown in FIG. 1, FIG. 2A, and is also illustrated below:

Sense Strand:
5′ mG-S-mA-fC-mA-mA-mA-mA-fA-fU-
fC-mC-fU-fC-mA-mC-mA-fA-mU-mA-mA-
mG-mC-mA-mG-mC-mC-[ademG-GaINAc]-
[ademA-GalNAc]-[ademA-GalNAc]-
[ademA-GaINAc]-mG-mG-mC-mU-mG-mC 3′
Hybridized to:
Antisense Strand:
5′ [MePhosphonate-4O-mU]-S-fU-
S-fA-S-mU-fU-mG-fU-fG-mA-fG-mG-
fA-mU-fU-mU-fU-mU-mG-fU-mC-S-mG-
S-mG 3′
Legend:
mX: 2′-O-methyl ribonucleotide
fx: 2′-fluoro-deoxyribonucleotide
[ademA-GalNAc]: 2′-modified-GalNAc adenosine
[ademG-GalNAc]: 2′-modified-GalNAc guanosine
[MePhosphonate-4O-mU]: 4′-O-
monomethylphosphonate-2′-O-methyl uridine
Linkages: “-” denotes phosphodiester
“-S-” denotes phosphorothioate

### Patient and Study Design

Data was provided from a global multicenter randomized placebo-controlled clinical trial which was conducted in 3 parts, a single ascending-dose (SAD) phase in healthy volunteers (HV; Group A, n=30), a single-dose (SD) phase in patients with Chronic Hepatitis B (CHB) and no treatment for their disease (NUC-naïve, Group B, with a single cohort B1, n=8), and a multiple ascending-dose (MAD) phase in 3 cohorts of patients with CHB who are NUC-suppressed (NUC-positive) (Group C, Cohorts C1, C2, C3, n=18 in total with 6 participants per cohort). Progression from the SAD phase to the first cohort in the MAD phase followed the Safety Review Committee (SRC) review of a minimum of 14 days postdose safety and tolerability data from all healthy volunteers (HV) in at least the first 2 SAD cohorts. Patients in the SAD HV cohorts (Group A) were assigned in a 2:1 ratio to receive 0.1, 1.5, 3, 6, or 12 mg/kg each as a single injection. In the SD Cohort B1, NUC-naïve patients were randomized 5:3 and received a single dose of 3 mg/kg. In the MAD Cohorts cohort C1, C2, and C3, patients who were NUC-suppressed (NUC-positive) were randomized 2:1 with 4 patients on active drug and 2 patients on placebo, and received 1.5, 3, or 6 mg/kg per dose, respectively, with a total of 4 doses (administered once every 4 weeks for 3 months).

Patients were eligible for treatment if they were 18 years of age or older, had been positive for hepatitis B surface antigen (HBsAg) for at least six months, had been HBeAg positive on two occasions within eight weeks prior to randomization, and had two episodes of elevated serum alanine aminotransferase (ALT) levels (at least twice the upper limit of normal (ULN)) on two occasions within eight weeks prior to randomization.

Patients were excluded from Group A if any of the following criteria applied: Medical Conditions 1. History of any medical condition that may interfere with the absorption, distribution or elimination of study drug, or with the clinical and laboratory assessments in this study, including (but not limited to); chronic or recurrent renal disease, functional bowel disorders (e.g., frequent diarrhea or constipation), GI tract disease, pancreatitis, seizure disorder, mucocutaneous or musculoskeletal disorder, history of suicidal attempt(s) or suicidal ideation, or clinically significant depression or other neuropsychiatric disorder requiring pharmacologic intervention 2. Poorly controlled or unstable hypertension; or sustained systolic BP >150 mmHg or diastolic BP >95 mmHg at Screen 3. History of diabetes mellitus treated with insulin or hypoglycemic agents 4. History of asthma requiring hospital admission within the preceding 12 months 5. Evidence of G-6-PD deficiency as determined by the Screen result at the central study laboratory 6. Currently poorly controlled endocrine conditions, except for thyroid conditions (hyper/hypothyroidism, etc.) where any pharmacologically treated thyroid conditions are excluded 7. A history of malignancy is allowed if the participant's malignancy has been in complete remission off chemotherapy and without additional medical or surgical interventions during the preceding 3 years 8. History of multiple drug allergies or history of allergic reaction to an oligonucleotide or GalNAc 9. History of intolerance to SC injection(s) or significant abdominal scarring that could potentially hinder study intervention administration or evaluation of local tolerability 10. Clinically relevant surgical history 11. History of persistent ethanol abuse (>40 g ethanol/day) or illicit drug use within the preceding 3 years 12. Clinically significant illness within the 7 days prior to the administration of study intervention 13. Donation of more than 500 mL of blood within the 2 months prior to administration of study intervention or plasma donation within 7 days prior to Screening 14. Significant infection or known inflammatory process ongoing at Screening (in the opinion of the Investigator) 15. History of chronic or recurrent urinary tract infection (UTI), or UTI within one month prior to Screening 16. Scheduled for an elective surgical procedure during the conduct of this study. Prior/Concomitant Therapy: 17. Use of prescription medications (except contraception medication for women) within 4 weeks prior to the administration of study intervention 18. Use of over-the-counter (OTC) medication or herbal supplements, excluding routine vitamins, within 7 days of first dosing, unless agreed as not clinically relevant by the Investigator and Sponsor. Prior/Concurrent Clinical Study Experience: 19. Has received an investigational agent within the 3 months prior to dosing or is in follow-up of another clinical study prior to study enrollment. Diagnostic assessments: 20. Seropositive for antibodies to human immunodeficiency virus (HIV), or hepatitis B virus (HBV), or hepatitis C virus (HCV), at Screening (historical testing may be used if performed within the 3 months prior to screening) 21. Alanine aminotransferase (ALT), aspartate aminotransferase (AST), gamma-glutamyl transferase (GGT), total bilirubin, alkaline phosphatase (ALP), or albumin outside of the reference range at Screening Visit 22. Complete blood count test abnormalities that are considered clinically relevant and unacceptable by the Investigator; hemoglobin (Hgb)<12.0 g/dL (equivalent to 120 g/L); platelets outside of the normal range 23. Hemoglobin A1C (HbA1C) >7% 24. Any other safety laboratory test result considered clinically significant and unacceptable by the Investigator. Other Exclusions: 25. Has undertaken, or plans to undertake, a significant change in exercise levels from 48 hours prior to entrance into the clinical research center until the end of study 26. Any condition that, in the opinion of the Investigator, would make the participant unsuitable for enrollment or could interfere with participation in or completion of the study.

For Groups B and C, participants with Hepatitis B were included in the study if they met the following criteria:
1. Age 18 (or age of legal consent, whichever is older) to 65 years inclusive, at the time of signing the informed consent 2. Chronic hepatitis B infection, documented by Table 3 and Table 4:

TABLE 3
Group C (NUC+)
Group C (NUC+)

| | Screening | | Serum |
|---|---|---|---|
| | HBsAg | ALT | IgM anti-HBc |
| HBeAG-positive | >1000 IU/mL | — | negative |
| HBeAG-negative | >500 IU/mL | — | negative |

TABLE 4
Group B (NUC naïve)
Group B (NUC naïve)

| | | | Serum | Serum |
|---|---|---|---|---|
| | | | IgM | HBV |
| | Screening HBsAg | ALT | anti-HBc | DNA |
| HBeAG- | >1000 IU/mL AND | ≥35 U/L (male) | negative | >2000 |
| positive | | ≥30 U/L (female) | | IU/mL |
| HBeAG- | >500 IU/mL | ≥35 U/L (male) | negative | >2000 |
| negative | | ≥30 U/L (female) | | IU/mL |

3. Clinical history compatible with compensated liver disease, with no evidence of cirrhosis: a. No history of bleeding from esophageal or gastrointestinal varices b. No history of ascites c. No history of jaundice attributed to chronic liver disease d. No history of hepatic encephalopathy e. No physical stigmata of portal hypertension—spider angiomata, etc. f. No previous liver biopsy, hepatic imaging study, or elastography result indicating cirrhosis (i.e., FibroScan>10.5 kPa). If Fibroscan has been performed within the last 6 months, results from that test may be used. 4. Continuously on NUC therapy (entecavir or tenofovir) for at least 12 weeks prior to the screening visit, with satisfactory tolerance and compliance (Group C). Participants should maintain a consistent dose throughout the course of the study or should be Treatment-naïve for hepatitis B (i.e., no previous antiviral therapy for hepatitis B or previous HBV NUC- or interferon-containing treatment; Group B) 5. Serum ALT at screening ≥35 U/L (males) or ≥30 U/L (females) for NUC-naïve (Group B) participants only. If liver biopsy is available within the last 6 months, histological evidence of immunoactive CHB is sufficient. NUC-experienced (Group C) participants are allowed to have ALT values within normal range. 6. 12-lead electrocardiogram (ECG) with no clinically significant abnormalities at Screening and predose on Day 1 (in the opinion of the Investigator) 7. Nonalcoholic Fatty Liver Disease is permissible. No other known cause of liver disease is acceptable. Weight: 8. Body Mass Index (BMI) within the range 18.0 to 35.0 kg/m2 (inclusive). Sex: 9. Male or female a. Male participants: A male participant must agree to use contraception, during the treatment period and following the last dose of study intervention for at least 12 weeks (single-dose administration in Group B) or 12 weeks (multiple-dose administration in Group C) and refrain from donating sperm during these periods. b. Female participants: A female participant is eligible to participate if she is not pregnant, not breastfeeding, and at least one of the following conditions applies: o Not a woman of child bearing potential (WOCBP) as defined in Appendix 4 or, depending on region a WOCBP who agrees to follow the contraceptive guidance during the treatment period and for at least 12 weeks after the dose of study intervention. Informed Consent: 10. The patient must be capable of giving signed informed consent, which includes compliance with the requirements and restrictions listed in the ICF and in this protocol. For Groups B and C, participants were excluded if any of the following criteria applied: Medical Conditions: 1. Has any clinically significant history or presence of poorly controlled or decompensated neurological, endocrine, cardiovascular, pulmonary, hematological, immunologic, psychiatric, metabolic, or other uncontrolled systemic disease, that may affect participation in the study 2. Presence of any concomitant medical or psychiatric condition or social situation that, in the opinion of the investigator, would make it difficult to comply with protocol requirements or put the participant at additional safety risk 3. Poorly controlled or unstable hypertension 4. Poorly controlled diabetes mellitus (serum HbA1c>8.0%) treated with insulin or hypoglycemic agents 5. Evidence of G-6-PD deficiency as determined by the screen result at the central study laboratory 6. Clinical history of hepatocellular carcinoma (HCC) 7. History of malignancy (other than HCC) is allowable if the malignancy has been in complete remission off chemotherapy and without additional medical or surgical interventions during the preceding 3 years 8. History of persistent ethanol abuse (>40 gm ethanol/day) or illicit drug use within the preceding 3 years 9. History of intolerance to SC injection(s) or significant abdominal scarring that could potentially hinder study intervention administration or evaluation of local tolerability 10. Receipt of a transfusion in the last 6 weeks prior to therapy or anticipated transfusions through the post-trial follow-up 11. Donated or lost >500 mL of blood within 2 months prior to Screening, or plasma donation within 7 days prior to Screening. Prior/Concomitant Therapy: 12. Antiviral therapy (other than entecavir or tenofovir in Group C) within 3 months of Screening or treatment with interferon in the last 3 years 13. Use within the last 6 months of (or an anticipated requirement for) anticoagulants, systemically administered corticosteroids, systemically administered immunomodulators, or systemically administered immunosuppressants 14. Use of prescription medication within 14 days prior to administration of study intervention that, in the opinion of the Investigator or the Sponsor, would interfere with study conduct 15. Depot injection or implant of any drug within 3 months prior to administration of study intervention, with the exception of injectable/implantable birth control. Prior/Concurrent Clinical Study Experience: 16. Has received an investigational agent within the 3 months prior to dosing or is in follow-up of another clinical study prior to study enrollment. Diagnostic assessments: 17. Systolic blood pressure >150 mmHg and a diastolic blood pressure of >95 mmHg after 10 minutes supine rest, at Screening 18. Hepatic transaminases (ALT or aspartate aminotransferase, AST) confirmed >7×ULN at Screening 19. History of persistent or recurrent hyperbilirubinemia, unless known Gilbert's Disease or Dubin-Johnson Syndrome 20. Seropositive for antibodies to HIV, HCV, or HDV. In participants with previous treatment for hepatitis C with direct-acting HCV medication and seropositivity for HCV, HCV RNA must be undetectable. 21. Hgb <12 g/dL (males) or <11 g/dL (females) 22. Serum albumin <3.5 g/dL at screening 23. Total WBC count <3,000 cells/μL or absolute neutrophil count (ANC)<1800 cells/μL at screening. 24. Platelet count ≤100,000 per L at screening 25. International normalized ratio (INR) or prothrombin time (PT) above the upper limit of the normal reference range (as per the testing laboratory reference range) at screening 26. Serum BUN or creatinine >ULN 27. Serum amylase or lipase >1.25×ULN 28. Serum alpha-fetoprotein (AFP) value >100 ng/mL. If AFP at screening is >ULN but <100 ng/mL, participant is eligible if a hepatic imaging study reveals no lesions suspicious of possible HCC 29. Any other safety laboratory test result considered clinically significant and unacceptable by the Investigator. Other Exclusions: 30. Has undertaken, or plans to undertake, a significant change in exercise levels from 48 hours prior to entrance into the clinical research center until the end of study 31. Any condition that, in the opinion of the Investigator, would make the participant unsuitable for enrollment or could interfere with participation in or completion of the study 32. Have any contraindications, as per local package insert, to entecavir or tenofovir (only for Group B).

HBVS-219 was prepared as a sterile formulation in water for injection. The unit dose strength was 195 mg/ml. The route of administration was by subcutaneous injection (thigh or abdomen). The HBVS-219 preparation was stored at 2° C. to 8° C. (inclusive) and protected from light and freezing temperatures. The HBVS-219 preparation was warmed to room temperature for approximately 1 hour (but no more than 4 hours) before administration. The maximum volume of a single subcutaneous injection did not exceed 0.8 mL. If the total volume for a single injection exceeded 0.8 mL, the dose was administered as two or more subcutaneous injections.

Serum HBsAg levels are correlated with covalently closed circular DNA (cccDNA) and intrahepatic HBV DNA and are increasingly used to predict and monitor treatment response to peg-interferon treatment. Efficacy (pharmacodynamics, PD) was assessed through the measurement of quantitative serum HBsAg, qualitative serum HBsAg, quantitative serum HBeAg, quantitative serum HBV DNA, quantitative serum HBV RNA, quantitative serum HBcrAg, and serum ALT.

Participants were followed every 28 days (±7 days) after the end of the treatment period until HBsAg level was <1 log 10 IU/mL below the Day 1 value (“conditional follow-up”). Note that, for the purpose of conditional follow-up, the treatment-assignment blind could be broken after the end of the treatment period for those participants who did not return to within 1 log 10 IU/mL of Day 1 HBsAg. The study was considered completed for participants who have undergone 6 months of conditional follow-up but still have not achieved a HBsAg level <1 log 10 IU/mL below their Day 1 value. These participants were offered an extension study for which they would have to consent.

Patients were monitored using a number of assays.

Quantitative Serum HBsAg (qHBsAg) Levels. Serum qHBsAg quantification was performed by Sonic Clinical Trials (SCT) using an Elecsys HBsAg II (Roche Diagnostics, Indianapolis, USA) device and its kits. This device utilizes an electro-chemiluminescence immunoassay (ECLIA) technique.

Qualitative Serum HBsAg levels and Anti-HBs. Serum qualitative HBsAg and Anti-HBs was assessed by SCT using the MODULAR® Analytics E170 (Roche Diagnostics, Indianapolis, USA) device and its kits. This device utilizes an ECLIA technique. Anti-HBs testing was performed in participants with undetectable HBsAg levels.

Quantitative Serum HBeAg levels. Quantitative HBeAg levels (in HBeAg-positive participants) was assessed by VIDRL (Victorian Infectious Diseases Research Laboratory) assay on LIAISON® (DiaSorin, S.p.A., Italy).

Qualitative Serum HBeAg Levels and Anti-HBe Serum qualitative HBeAg and Anti-HBe was assessed on a Roche Cobas® analyzer and its kits. This device utilizes an ECLIA technique. Anti-HBe testing was performed in participants with undetectable HBeAg levels.

Quantitative Serum HBV DNA levels. Quantitative HBV DNA levels was assessed via the Cobas® 4800 HBV DNA polymerase chain reaction (PCR) assay, version 2.0 (Roche Diagnostics, Indianapolis, USA).

Quantitative Serum HBV RNA Levels. Quantitative HBV RNA levels was assessed by quantitative real-time-PCR (qRT-PCR) assay performed in LC480 II real-time PCR instrument (Roche Diagnostics, Indianapolis, USA).

Quantitative HBcrAg. Quantitative HBcrAg levels in blood was assessed by the LumiPulse® chemiluminesence assay (Fujirebio, USA).

Alanine Aminotransferase Levels and Flares. Early on treatment ALT increases (flares), defined as substantial ALT elevations (>3 times Baseline value and >10 ULN) without declining hepatic synthetic function (decreasing albumin) or declining excretory function (increasing bilirubin) may be evidence of an immune response against HBV and its downstream mechanisms. Higher ALT levels may reflect a more robust immune clearance of HBV and, therefore, a higher chance of HBV-DNA loss and HBeAg seroconversion. ALT levels will be measured as part of the clinical chemistry panel at the visits indicated in the schedule of activities. Any participant experiencing ALT increase (flare) was further followed.

Sample sizes of 30 participants in Group A, 8 participants in Group B, and 18 participants in Group C provide an assessment of the safety profile of HBVS-219 in healthy adults and participants with hepatitis B, respectively. The efficacy assessments in Group B and Group C participants provide data on single- and multiple-dose-related efficacy effects, such as reductions in quantitative serum HBsAg and HBV DNA levels.

For all Figures BL is baseline and the time in days on the X axis to the right of BL is from the first administration of HBVS-219 or placebo.

Group C results (NUC-positive patients) are provided below. Patients in Group C cohorts C1, C2 and C3 received up to four HBVS-219 doses of 1.5 mg/kg (C1), 3 mg/kg (C2) and 6 mg/kg (C3) respectively. A summary of the corresponding fixed dose the patients received is provided in Table 5 below.

TABLE 5
Summary of HBVS-219 patient fixed dosage (mg) for group C

| | | Cohort C1— | Cohort C2— | Cohort C3— |
|---|---|---|---|---|
| Visit | Statistics | 1.5 mg/kg | 3 mg/kg | 6 mg/kg |
| Day 1 | n | 4 | 4 | 3 |
| | Mean | 89.8 | 189.5 | 403.4 |
| | Standard | 28.28 | 35.58 | 32.66 |
| | Deviation | | | |
| | Median | 76.3 | 189.9 | 391.2 |
| | Minimum | 74 | 149 | 379 |
| | Maximum | 132 | 229 | 440 |
| Day 29 | n | 4 | 4 | 3 |
| | Mean | 89.5 | 190.7 | 397.2 |
| | Standard | 28.44 | 36.54 | 34.16 |
| | Deviation | | | |
| | Median | 76.3 | 191.3 | 382.8 |
| | Minimum | 74 | 150 | 373 |
| | Maximum | 132 | 230 | 436 |
| Day 57 | n | 4 | 4 | 2 |
| | Mean | 89.6 | 191.5 | 381.0 |
| | Standard | 28.55 | 38.91 | 13.58 |
| | Deviation | | | |
| | Median | 76.4 | 188.7 | 381.0 |
| | Minimum | 73 | 152 | 371 |
| | Maximum | 132 | 237 | 391 |
| Day 85 | n | 4 | 4 | 2 |
| | Mean | 90.0 | 193.5 | 381.3 |
| | Standard | 29.05 | 36.98 | 12.30 |
| | Deviation | | | |
| | Median | 76.3 | 191.0 | 381.3 |
| | Minimum | 74 | 156 | 373 |
| | Maximum | 134 | 236 | 390 2 |

Dosage (mg) administered is calculated based on body weight (kg) at dosing * dose level (mg/kg). The table presents an average of the participant's mean dosage administered at each visit (or single dose). Only participants in treatment arms are included in the table & listing.

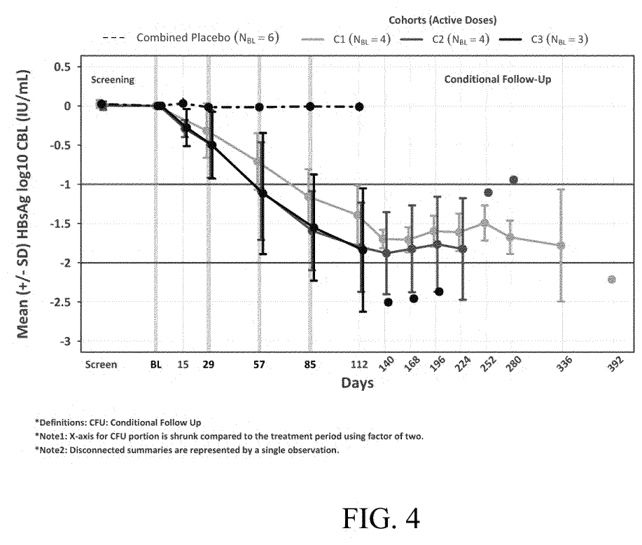

FIG. 4 shows mean changes in HBsAg change from baseline, CBL, (IU/ml) for NUC-positive HBV patients (group C). NUC-positive HBV patients (continuously on NUC therapy, entecavir or tenofovir, for at least 12 weeks prior to the screening visit) were given up to 4 rounds of HBVS-219 on days 1, 29, 57 and 85. Dashed black line is placebo across cohorts (n=6), light grey solid line is 1.5 mg/kg per dose HBVS-219 cohort C1 (n=4), medium grey solid line is 3 mg/kg per dose HBVS-219 cohort C2 (n=4), black solid line is 6 mg/kg per dose HBVS-219 cohort C3 (n=3). Y axis shows mean (+/− Standard deviation) HBsAg log 10 change from baseline, CBL, (IU/mL). Patients were conditionally followed up after treatment (CFU). The X-axis shows the time in days and the CFU portion is shrunk compared to the treatment period using a factor of two (for enhanced visualization). Error bars show standard deviation. Disconnected summaries (dots) are represented by a single observation (therefore no error bars) when the trial had only one subject. All patient groups treated with HBVS-219 showed a mean HBsAg reduction (that increased over time) in the four-week period after each administration of HBVS-219. Patients monitored in groups C2 and C3 show long term reduction in HBsAg up to over 2 log fold between days 112 and 392. Changes of HBsAg levels are shown from the baseline in Group C.

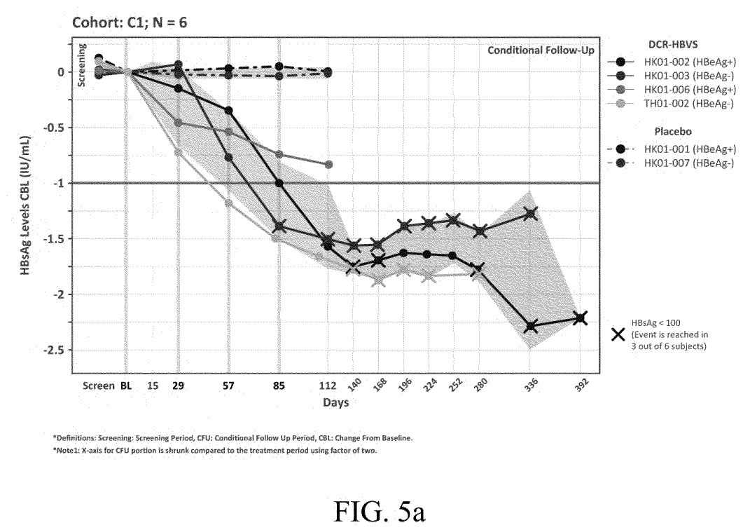

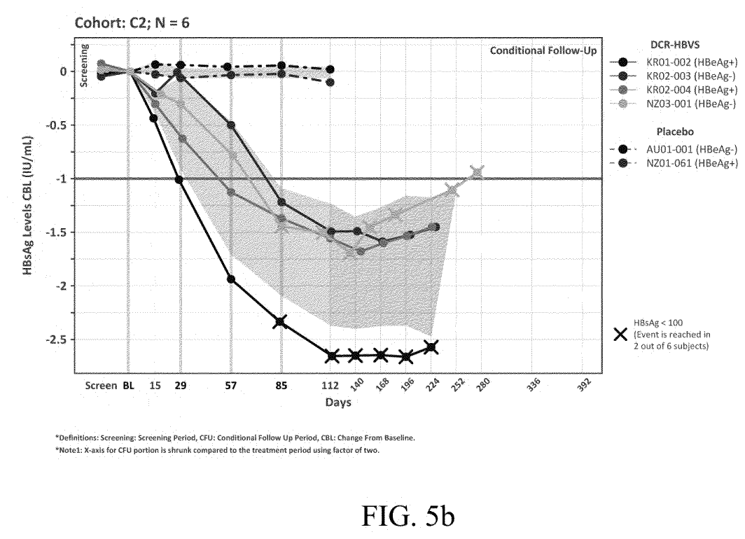

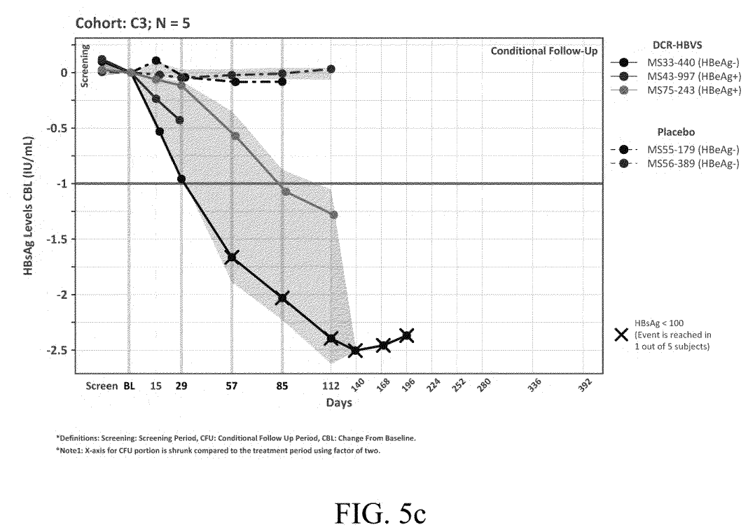

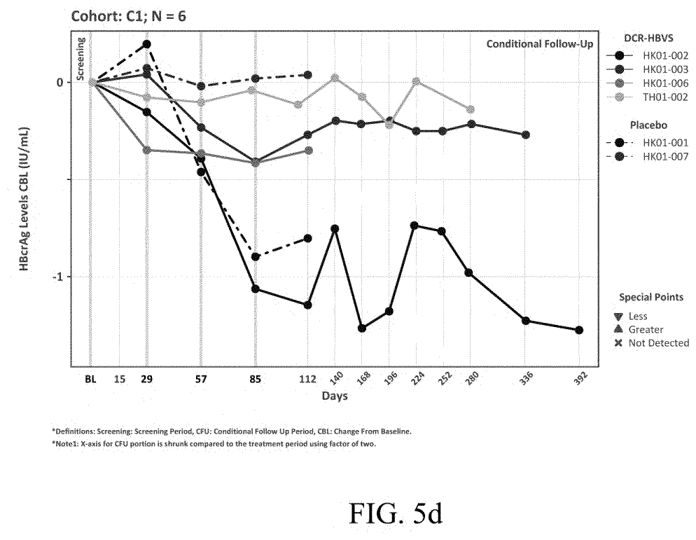

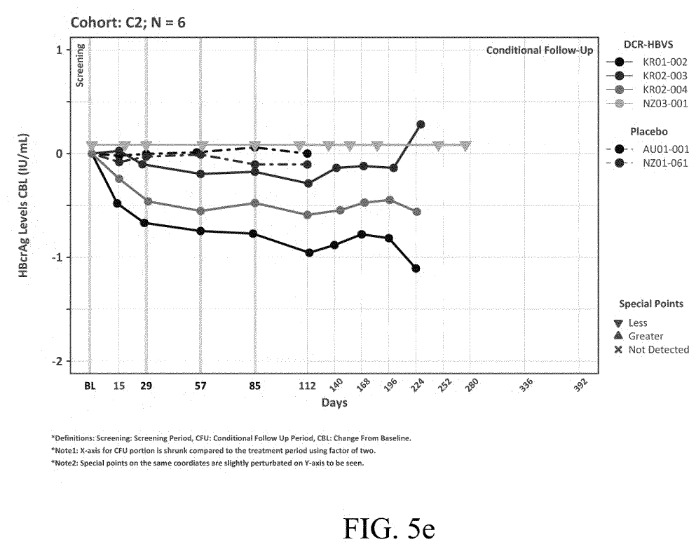

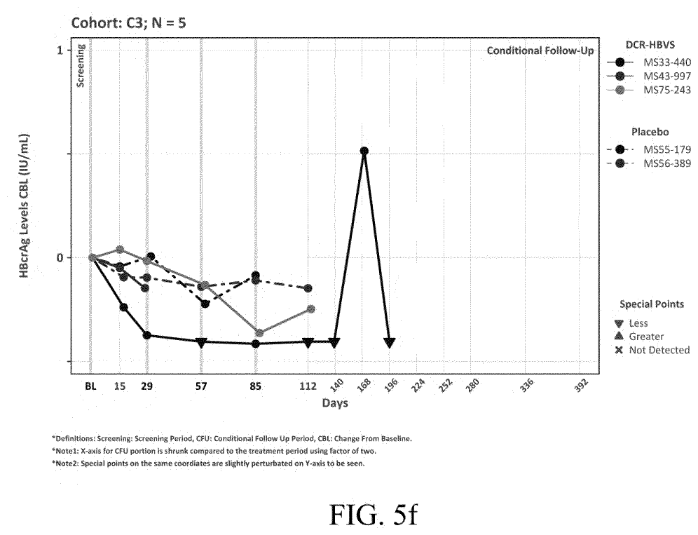

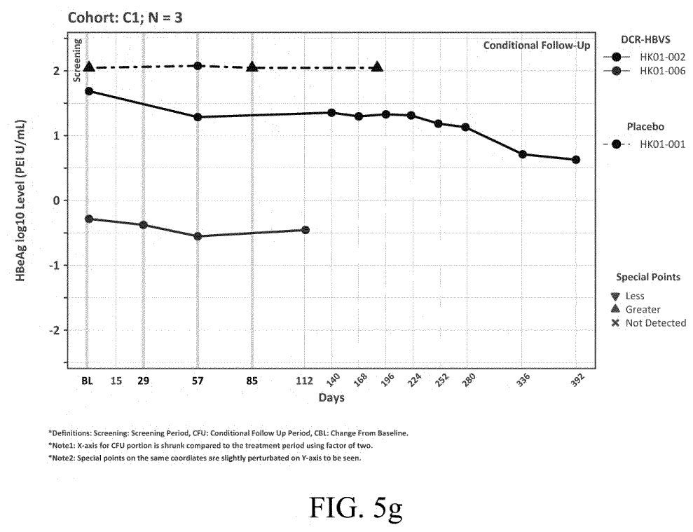

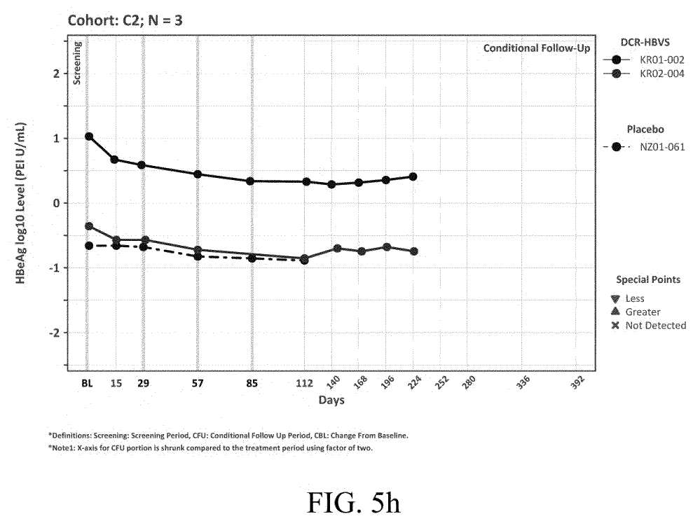

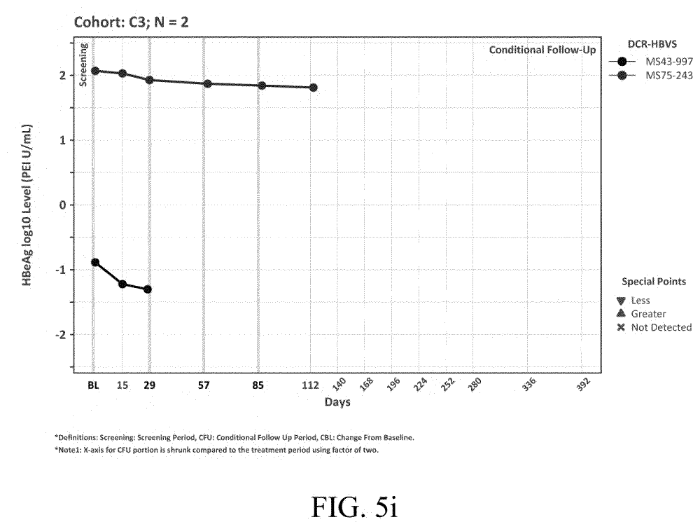

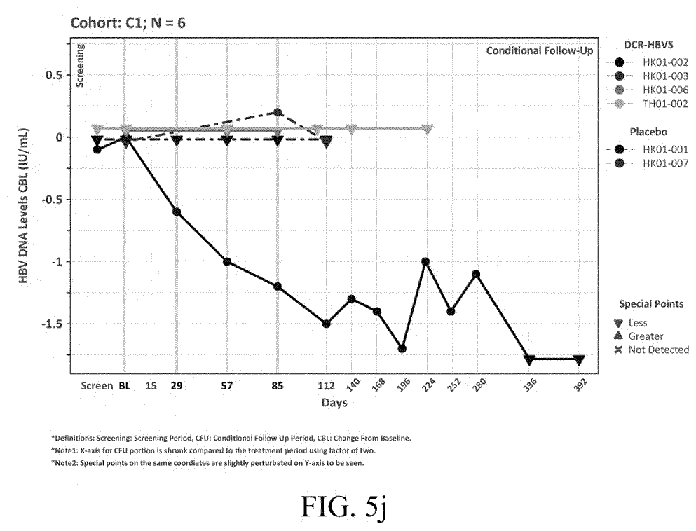

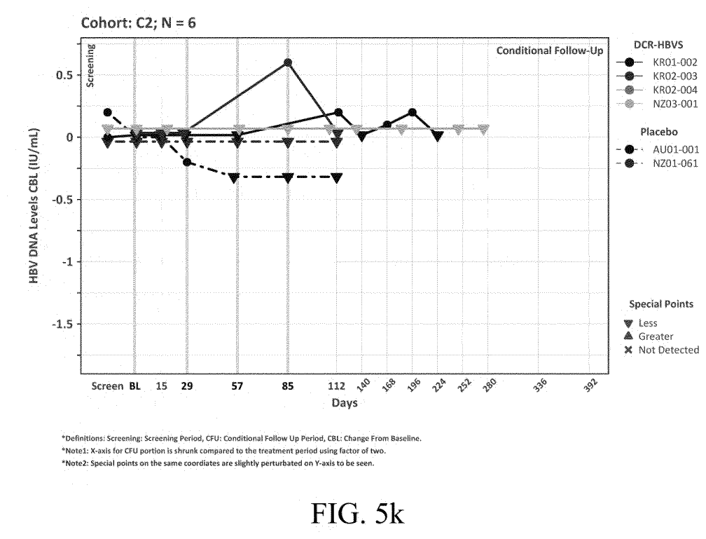

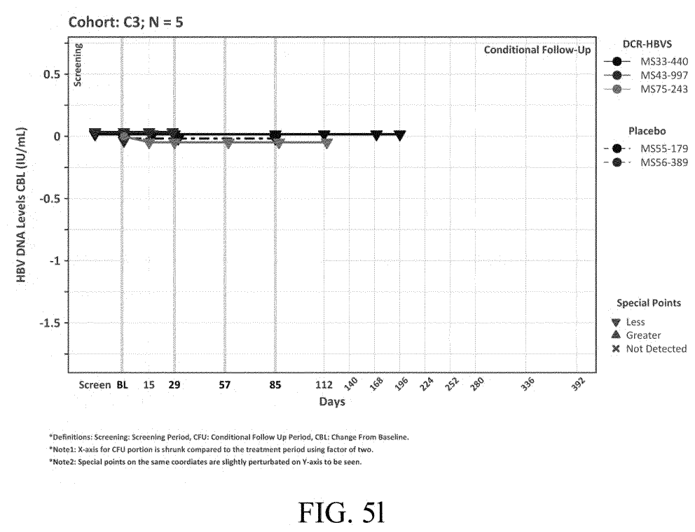

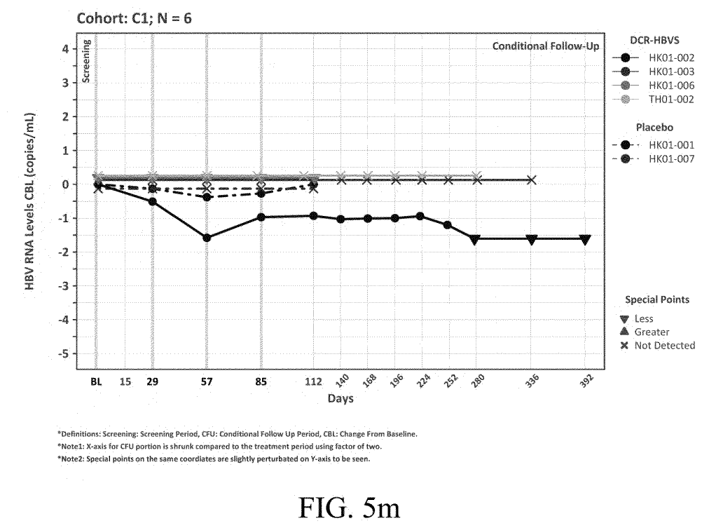

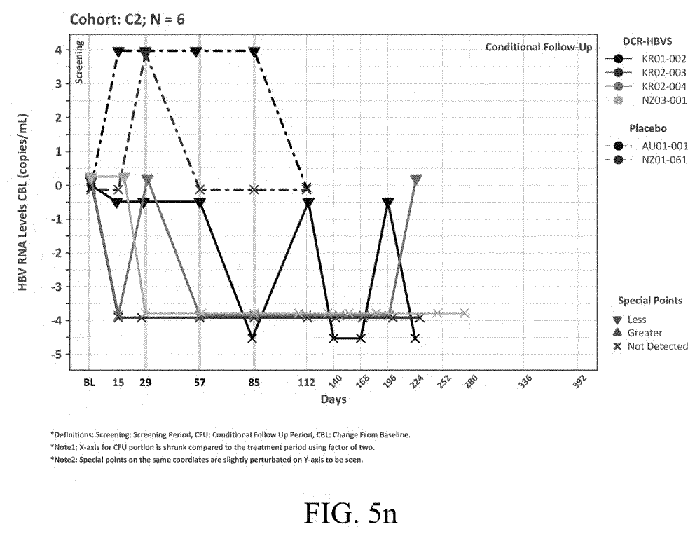

FIG. 5a shows individual patient changes in HBsAg levels from baseline readings in HBV patients treated with 1.5 mg/kg HBVS-219 per round, cohort C1 (averaged as light grey solid line in FIG. 4) against the cohort C1 placebo controls. In FIG. 5a, HBSV-219 treated patients are shown by solid grey and black lines (n=4), placebo controls are shown by dashed lines (n=2). Y axis is HBsAg levels CBL (IU/mL). X axis is time in days with a conditional follow up period (CFL) after treatment that is shrunk compared to the treatment period using a factor of two (for enhanced visualisation). All patients treated with HBVS-219 measured between 112 and 392 days show persistent reductions in HBsAg levels of more than 1 log below the baseline (visualised by background shaded area after 112 days). In FIG. 5a the X over the dot (point) indicates that for this particular value, the total HBsAg had reached an absolute level of less than 100 IU/ml (reached in 3 out of 6 patients). Data are provided adjusted for the average weight in cohort C1.

FIG. 5b shows individual changes of HBsAg levels from baseline in HBV patients treated with 3 mg/kg HBVS-219 per round, cohort C2 (averaged as dark grey solid line in FIG. 4) against the cohort C2 placebo controls. In FIG. 5b, HBVS-219 treated patients are shown by solid grey and black lines (n=4) and placebo controls are shown by dashed lines (n=2). Y axis is HBsAg levels CBL (IU/mL). X axis is time in days with a conditional follow up period (CFL) after treatment that is shrunk compared to the treatment period using a factor of two (for enhanced visualization). An X over the dot (point) shows HBsAg <100 IU/mL. The HBsAg <100 IU/mL event was reached in 2 out of 6 patients. A clear reduction in HBsAg is observed in all treated patients with a 1 log reduction in all monitored patients at day 85 that persists for all remaining measurements made up to 252 days. In FIG. 5b the results were adjusted for the average weight in the cohort tested, cohort C2.

FIG. 5c shows individual changes of HBsAg levels from baseline in HBV patients treated with 6 mg/kg HBVS-219 per round, cohort C3, (averaged as black solid line in FIG. 4) and the cohort C3 placebo control. In FIG. 5c HBVS-219 patients are shown by grey and black solid lines (n=3) and placebos (n=2) are shown by dashed lines. The results were adjusted for the average weight in C3. Y axis is HBsAg levels CBL (IU/mL). X axis is time in days with a conditional follow up period (CFL) after treatment that is shrunk compared to the treatment period using a factor of two (for enhanced visualization). X over dot (point) shows HBsAg <100 IU/mL (reached in 1 out of 5 patients). The two patients (MS33-440 and MS43-997) measured up to 112 days show a more than 1-fold reduction in HBsAg at 85 days with the reduction persisting until the final measurement point for each patient.

FIG. 5d shows HBcrAg changes in Group C1 (NUC positive) individual treated patients, change from baseline (CBL). Data are normalised to zero by weight. Y axis is HBcrAg level CBL (IU/mL). X axis is time in days. Conditional follow up period days 112-392 (shrunk compared to the treatment period using factor of two). Solid black and grey lines are HBVS-219 treated patients (n=4). Dashed black and grey lines are placebo. (n=2). Special points: downwards triangle (less than detectable limit), upwards triangle (above detectable limit), x (not detected).

FIG. 5e shows HBcrAg changes in Group C2 (NUC positive) individual treated patients, change from baseline (CBL). Data are normalised to zero by weight. Y axis is HBcrAg level CBL (IU/mL). X axis is time in days. Conditional follow up period days 112-392 (shrunk compared to the treatment period using factor of two). Solid black and grey lines are HBVS-219 treated patients (n=4). Dashed black and grey lines are placebo (n=2). Special points: downwards triangle (less than detectable limit), upwards triangle (above detectable limit), x (not detected). Special points on the same coordinates are slightly perturbated on Y axis to be seen.

FIG. 5f shows HBcrAg changes in Group C3 (NUC positive) individual treated patients, change from baseline (CBL). Data are normalised to zero by weight. Y axis is HBcrAg level CBL (IU/mL). X axis is time in days. Conditional follow up period days 112-392 (shrunk compared to the treatment period using factor of two). Solid black and grey lines are HBVS-219 treated patients (n=3). Dashed black and grey lines are placebo (n=2). Special points: downwards triangle (less than detectable limit), upwards triangle (above detectable limit), x (not detected). Special points on the same coordinates are slightly perturbated on Y axis to be seen.

FIG. 5g shows HBeAg changes in Group C1 (NUC positive) individual treated patients, CBL. Y axis is HBeAg log 10 level (PEI IU/mL). X axis is time in days. Conditional follow up period days 112-392 (shrunk compared to the treatment period using factor of two). Solid black and grey lines are HBVS-219 treated patients (n=2). Dashed black line is placebo (n=1). Special points: downwards triangle (less than detectable limit), upwards triangle (above detectable limit), x (not detected). Special points on the same coordinates are slightly perturbated on Y axis to be seen.

FIG. 5h shows HBeAg changes in Group C2 (NUC positive) individual treated patients, CBL. Y axis is HBeAg log 10 level (PEI IU/mL). X axis is time in days. Conditional follow up period days 112-392 (shrunk compared to the treatment period using factor of two). Solid black and grey lines are HBVS-219 treated patients (n=2). Dashed black line is placebo (n=1). Special points: downwards triangle (less than detectable limit), upwards triangle (above detectable limit), x (not detected). Special points on the same coordinates are slightly perturbated on Y axis to be seen.

FIG. 5i shows HBeAg changes in Group C3 (NUC positive) individual treated patients, CBL. Y axis is HBeAg log 10 level (PEI IU/mL). X axis is time in days. Conditional follow up period days 112-392 (shrunk compared to the treatment period using factor of two). Solid black and grey lines are HBVS-219 treated patients (n=2). Special points: downwards triangle (less than detectable limit), upwards triangle (above detectable limit), x (not detected). Special points on the same coordinates are slightly perturbated on Y axis to be seen.

FIG. 5j shows HBV DNA changes in group C1 (NUC positive) individual treated patients, CBL. Y axis is HBV DNA level CBL (IU/mL). X axis is time in days. Data are normalised to zero by weight. Conditional follow up period days 112-392. Solid black and grey lines are HBVS-219 treated patients (n=4). Dashed black and grey lines are placebo (n=2). Conditional follow up period days 112-392 (shrunk compared to the treatment period using factor of two). Special points on the same coordinates are slightly perturbated on Y axis to be seen. Special points: downwards triangle (less than detectable limit), upwards triangle (above detectable limit), x (not detected).

FIG. 5k shows HBV DNA changes in group C2 (NUC positive) individual treated patients, CBL. Y axis is HBV DNA level CBL (IU/mL). X axis is time in days. Data are normalised to zero by weight. Conditional follow up period days 112-392. Solid black and grey lines are HBVS-219 treated patients (n=4). Dashed black and grey lines are placebo (n=2). Conditional follow up period days 112-392 (shrunk compared to the treatment period using factor of two). Special points on the same coordinates are slightly perturbated on Y axis to be seen. Special points: downwards triangle (less than detectable limit), upwards triangle (above detectable limit), x (not detected).

FIG. 5l shows HBV DNA changes in group C3 (NUC positive) individual treated patients, CBL. Y axis is HBV DNA level CBL (IU/mL). X axis is time in days. Data are normalised to zero by weight. Conditional follow up period days 112-392. Solid black and grey lines are HBVS-219 treated patients (n=3). Dashed black and grey lines are placebo (n=2). Conditional follow up period days 112-392 (shrunk compared to the treatment period using factor of two). Special points on the same coordinates are slightly perturbated on Y axis to be seen. Special points: downwards triangle (less than detectable limit), upwards triangle (above detectable limit), x (not detected).

FIG. 5m shows HBV RNA changes in group C1 (NUC positive) individual treated patients, CBL. Y axis is HBV RNA level CBL (copies/mL). Data are normalised to zero by weight. X axis is time in days. Conditional follow up period days 112-392 (shrunk compared to the treatment period using factor of two). Solid black and grey lines are HBVS-219 treated patients (n=4). Dashed black and grey lines are placebo (n=2). Special points on the same coordinates are slightly perturbated on Y axis to be seen. Special points: downwards triangle (less than detectable limit), upwards triangle (above detectable limit), x (not detected).

FIG. 5n shows HBV RNA changes in group C2 (NUC positive) individual treated patients, CBL. Y axis is HBV RNA level CBL (copies/mL). X axis is time in days. Data are normalised to zero by weight. Conditional follow up period days 112-392 (shrunk compared to the treatment period using factor of two). Solid black and grey lines are HBVS-219 treated patients (n=4). Dashed black and grey lines are placebo (n=2). Special points on the same coordinates are slightly perturbated on Y axis to be seen. Special points: downwards triangle (less than detectable limit), upwards triangle (above detectable limit), x (not detected).

FIG. 5o shows HBV RNA changes in group C3 (NUC positive) individual treated patients, CBL. Y axis is HBV RNA level CBL (copies/mL). X axis is time in days. Data are normalised to zero by weight. Conditional follow up period days 112-392 (shrunk compared to the treatment period using factor of two). Solid black and grey lines are HBVS-219 treated patients (n=3). Dashed black and grey lines are placebo (n=2). Special points on the same coordinates are slightly perturbated on Y axis to be seen. Special points: downwards triangle (less than detectable limit), upwards triangle (above detectable limit), x (not detected).

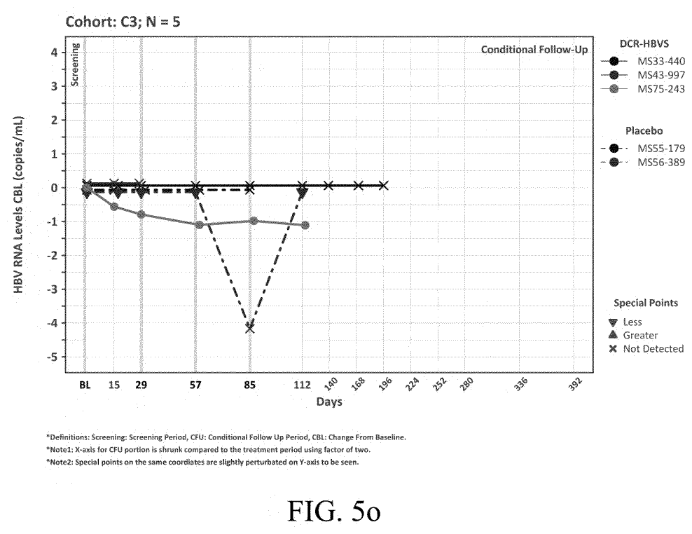

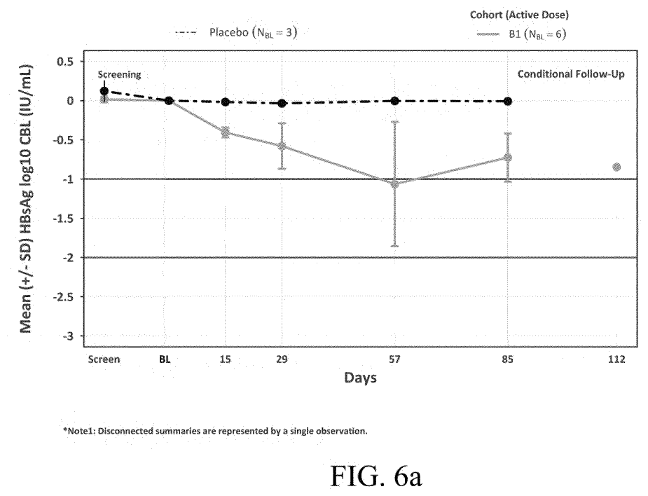

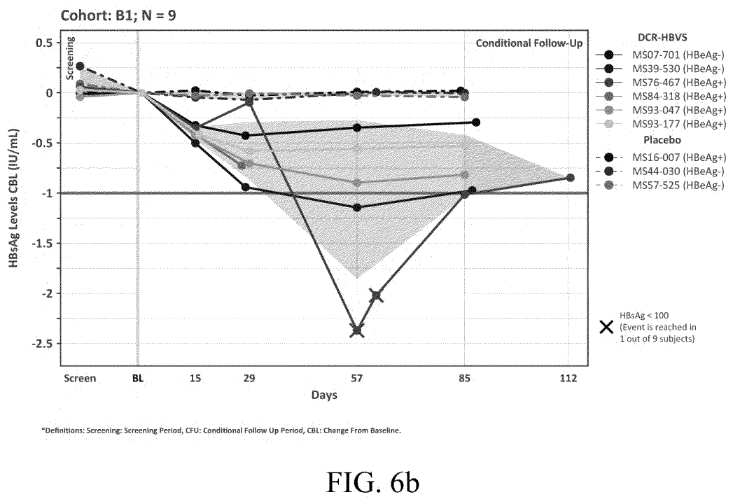

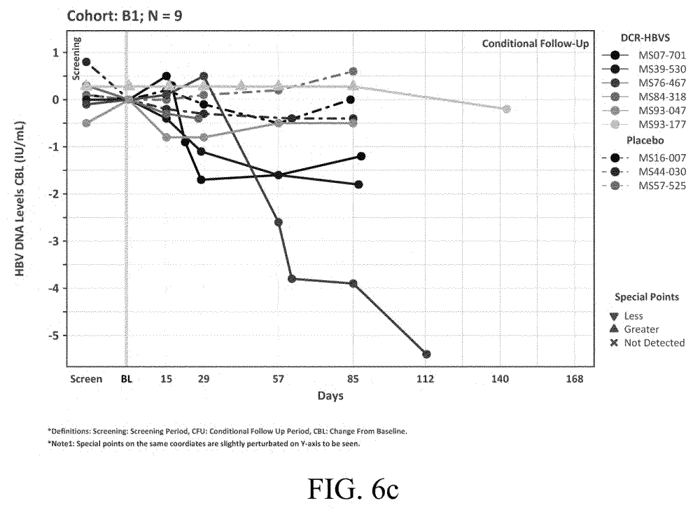

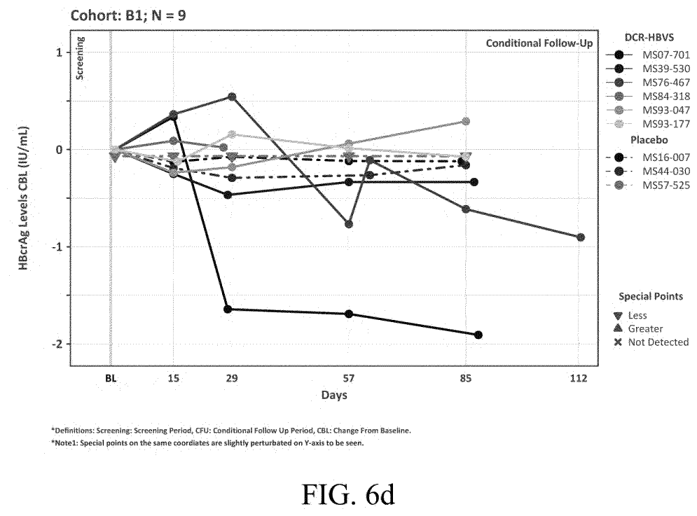

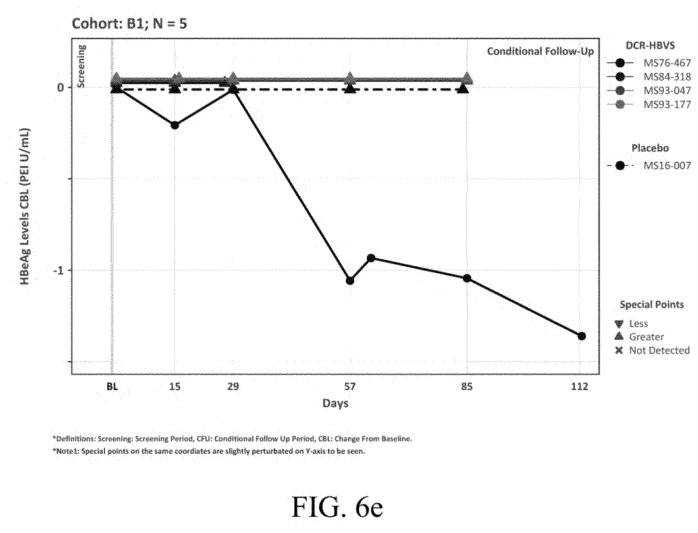

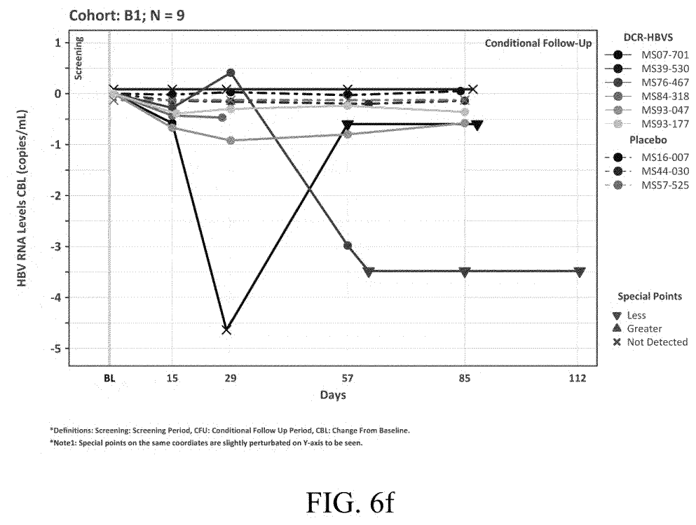

FIGS. 6a-9b show the results of the monotherapy NUC-naïve HBV patients (group B, cohort B1). NUC-naïve patients had no previous antiviral therapy for hepatitis B or previous HBV NUC or interferon-containing treatment. Patients were given on the first day (BL) a single dose of 3 mg/kg HBVS-219 (n=6) or placebo (n=3). All references in the Figures to DCR-HBVS refer to the administration of HBVS-219.

A summary of the corresponding fixed dose the patients received in group B is provided in Table 6 below.

TABLE 6
Summary of HBVS-219 patient fixed dosage (mg) for group B

| Visit | Statistics | Cohort B1—3 mg/kg |
|---|---|---|
| Day 1 | n | 6 |
| | Mean | 255.8 |
| | Standard | 35.43 |
| | Deviation | |
| | Median | 262.4 |
| | Minimum | 205 |
| | Maximum | 297 |

Dosage (mg) administered is calculated based on body weight (kg) at dosing * dose level (mg/kg). The table presents an average of the participant's mean dosage administered at each visit (or single dose). Only participants in treatment arms are included in the table & listing.

FIG. 6a shows mean changes in HBsAg from the baseline (CBL) of treatment NUC-naïve HBV patients treated with a single dose monotherapy treatment of 3 mg/kg HBVS-219 or placebo for the entire group B. Y axis is mean (+/−SD) HBsAg log 10 CBL (IU/mL). X axis is time in days following administration. Grey solid line is 3 mg/kg HBVS-219 treated average (n=6). Black dashed line is placebo treated average (n=3). Disconnected summaries are represented by a single observation (therefore no standard deviation).

FIG. 6b shows reduction in HBsAg levels, CBL (IU/mL) in NUC-naïve HBV individual patients treated with HBVS-219 or placebo for group B. Y axis is HBsAg levels CBL (IU/mL). X axis is time in days with a conditional follow up period 85 to 112 days. X over dot (point) shows HBsAg <100 (IU/mL), which was reached in 1 of 9 patients. Data are normalized by weight. All measured HBVS-219 treated patients show a reduction of HBsAg levels by 0.5 log at 57 days onwards. The reduction relative to the baseline over the measurement period is maintained. A reduction in HBsAg was seen in 6 of 6 (100%) of patients treated with a single dose of HBVS-219.

FIG. 6c shows the individual changes in HBV DNA in NUC-naïve HBV individual patients treated with HBVS-219 (n=6, grey and black solid lines) or placebo (n=3, dashed lines) for group B. Y axis is HBV DNA levels CBL (IU/mL). X axis is time in days with a conditional follow up period 85 to 168 days. Data are normalized by weight. Special points on the same coordinates are slightly perturbated on Y axis to be seen. Special points: downwards triangle (less than detectable limit), upwards triangle (above detectable limit), x (not detected). HBV DNA reduction is observed in most HBVS-219 treated patients in Cohort B1. The placebo patients show relatively stable HBV DNA levels through time. One patient (MS76-467) surprisingly shows a more than 5 log reduction in HBV DNA. FIG. 6c demonstrates the general reduction of HBV DNA seen upon monotherapy treatment with HBVS-219.

FIG. 6d shows individual changes in HBcrAg levels CBL (IU/ml) in cohort B1. Y axis is HBcrAg CBL (IU/mL). X axis is time in days with a conditional follow up period 85 to 112 days. Data are normalized by weight. Special points on the same coordinates are slightly perturbated on Y axis to be seen. Special points: downwards triangle (less than detectable limit), upwards triangle (above detectable limit), x (not detected). HBVS-219 treated patients shown in solid grey and black lines (n=6), placebos shown in dashed lines (n=3). FIG. 6d shows a reduction of HBcrAg levels from baseline upon HBVS-219 administration. Three out of six HBVS-219 treated patients (MS07-701, MS39-530 and MS76-467) show a reduction of HBcrAg levels from baseline levels on or after 85 days.

FIG. 6e shows individual changes in HBeAg levels upon HBVS-219 administration in cohort B1 (only patients who were e+ at baseline are shown). Y axis is HBeAg levels CBL (PEI IU/mL). X axis is time in days with a conditional follow up period after 85 to 112 days. Data are normalized by weight. Special points on the same coordinates are slightly perturbated on Y axis to be seen. Special points: downwards triangle (less than detectable limit), upwards triangle (above detectable limit), x (not detected). HBVS-219 treated patients shown in solid grey and black lines (n=4), placebo shown in dashed line (n=1). FIG. 6e demonstrates HBeAg reduction upon drug administration. Patients with greater than 100 PEI U/ml may have had reductions. There was an HBeAg reduction in at least one HBVS-219 treated patient (MS76-467).

FIGS. 6d and 6e provide additional data supporting efficacy of a monotherapy or run-in monotherapy phase with a reduction of HBcrAg levels and HBeAg levels. The other patients had levels above the limit of quantification (upwards triangle) following a single injection of HBVS-219 in treatment-naïve patients.

FIG. 6f shows HBV RNA changes in Group B (NUC naïve) individual treated patients, change from baseline (CBL). Data are normalised to zero by weight. Y axis is HBV RNA levels CBL (copies/mL). X axis is time in days. Conditional follow up period days 85-112. Solid black and grey lines are HBVS-219 treated patients (n=6). Dashed black and grey lines are placebo (n=3). Special points on the same coordinates are slightly perturbated on Y axis to be seen. Special points: downwards triangle (less than detectable limit), upwards triangle (above detectable limit), x (not detected).

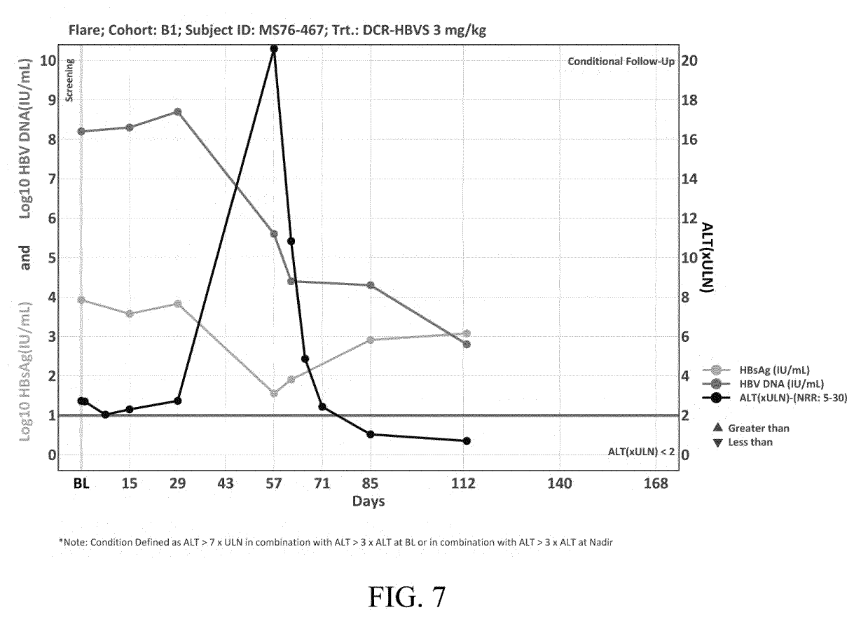

FIG. 7 shows changes in group B patient MS76-467, a NUC-naïve patient treated with 3 mg/kg HBVS-219. MS76-467 showed a 5 log reduction in levels of HBV DNA (dark grey solid line, scale on left hand side Y axis −log 10 HBV DNA IU/ML) at 113 days after HBVS-219 administration, a reduction in HBsAg (light grey solid line, scale on left hand side Y axis −log 10 HBsAg IU/mL) by 1 log after 113 days and an increase in ALT levels (indicative of a positive flare) between days 29 and 71 (black solid line, scale on right hand side Y axis −ALT (xULN)). An ALT positive flare is defined in FIG. 7 as ALT 7×ULN in connection with ALT >3×ALT at BL or in combination with ALT >3×ALT at Nadir. Special symbols: upwards triangle (greater than detection limit), downwards triangle (less than detection limit). Conditional follow up period 85 days to 168 days.

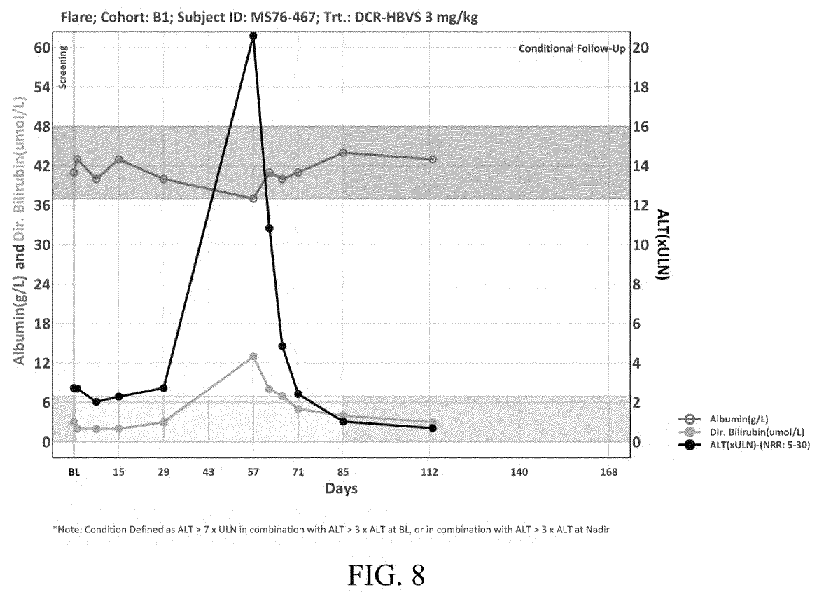

FIG. 8 shows the liver function as measurements of bilirubin (light grey solid line—filled in points, measured in umol/L) and albumin (dark grey solid line—non filled in points, measured in g/L) from group B patient MS76-467 which remained stable over the measurement period. FIG. 8 demonstrates that liver synthetic and excretory function was preserved through the direct measurement of bilirubin and albumin which stayed within the normal reference range (shaded areas surrounding bilirubin and albumin measurement lines represent normal ranges respectively) except for a brief increase in direct bilirubin at Day 57. ALT changes are provided as the black solid line (xULN), in accordance with FIG. 7). An ALT positive flare is defined in FIGS. 7 and 8 as ALT 7 xULN in connection with ALT >3×ALT at BL or in combination with ALT >3×ALT at Nadir. Conditional follow up period 85 days to 168 days. FIG. 8 demonstrates that liver function was preserved and in combination with FIG. 7 supports the flare in patient MS76-467 being a positive flare.

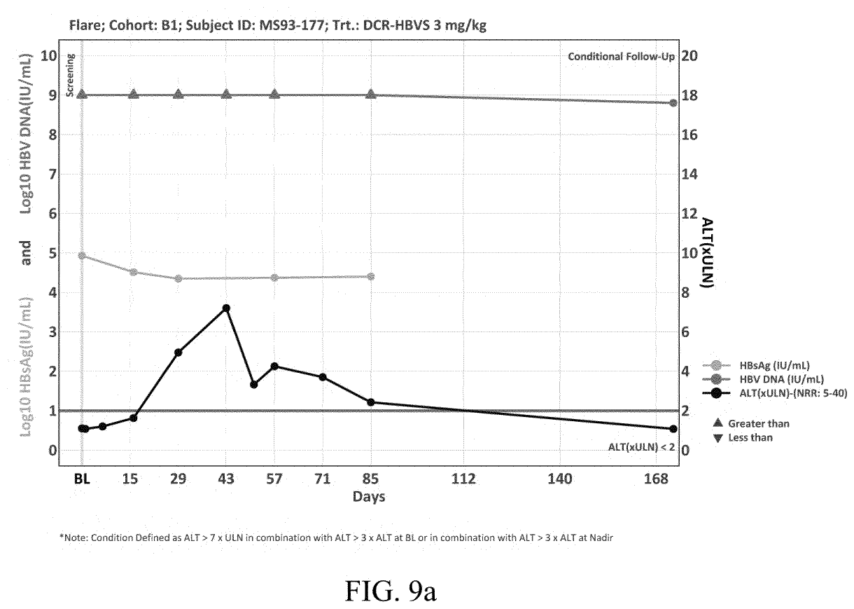

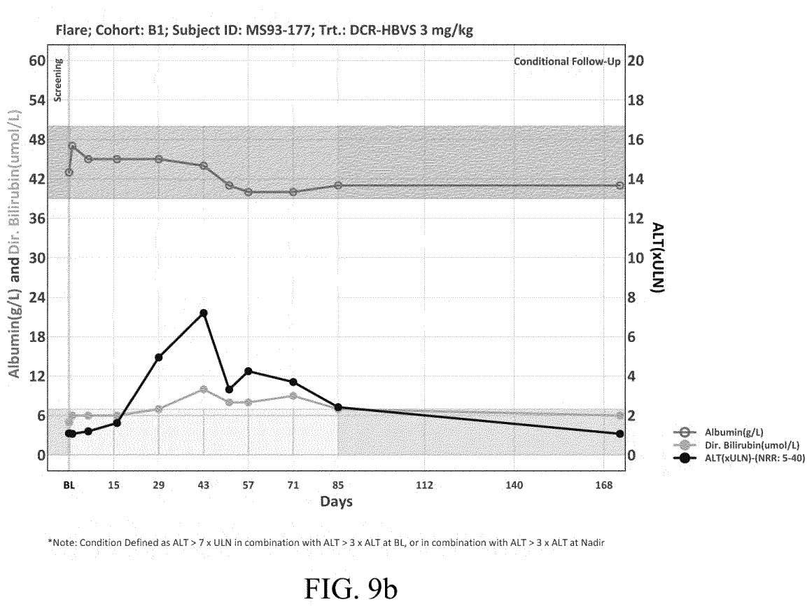

FIG. 9a shows changes in group B patient MS93-177, a NUC-naïve patient treated with 3 mg/kg HBVS-219. MS93-177 showed no reduction in levels of HBV DNA (dark grey solid line, scale on left hand side Y axis—log 10 HBV DNA (IU/mL)), a slight reduction in HBsAg (light grey solid line, scale on left hand side Y axis—log 10 HBsAg (IU/mL)) and an increase in ALT levels (black solid line, scale on right hand side axis—(xULN)). An ALT positive flare is defined in FIG. 9a as ALT 7 xULN in connection with ALT >3×ALT at BL or in combination with ALT >3×ALT at Nadir. Special symbols: upwards triangle (greater than detection limit), downwards triangle (less than detection limit). Conditional follow up period 85 days to 168 days.

FIG. 9b shows largely stable liver function in cohort B1 patient MS93-177. Albumin measured as dark grey solid line with non filled in points (Y axis on left hand side, (g/L)), bilirubin measured as light grey solid line (Y axis on left hand side, (umol/L)). There was a very small increase in direct bilirubin at days 43 to 71. ALT levels measured as black solid line (scale on right hand side, (xULN)). An ALT positive flare is defined in FIG. 9b as ALT 7 xULN in connection with ALT >3×ALT at BL or in combination with ALT >3×ALT at Nadir. FIG. 9b further supports MS93-177 having a positive flare. FIG. 9b demonstrates that liver synthetic and excretory function was preserved through the direct measurement of bilirubin and albumin which largely stayed within the normal reference range (shaded areas surrounding bilirubin and albumin measurement lines represent normal ranges respectively).

FIG. 10 shows a time dependent overview of Injection Site-Related Adverse Events for group B and group C patients. FIG. 10 shows that HBVS-219 is safe. FIG. 10 is a subject level plot for the number of adverse events (AEs) associated with injection sites. Y axis is number of injection sites. X axis is time in days with doses indicated. Injection Site Reaction (ISR) defined as signs or symptoms at the injection site reported within 4 hours post dose administration. Grade 1—tenderness with or without associated symptoms (for example warmth, erythema, itching). Grade 2—pain, lipodystrophy, edema, phlebitis. Grade 3—ulceration or necrosis, severe tissue damage, operative intervention indicated. Grade 4—life threatening consequences, urgent intervention indicated. Grade 5—death. After 4 hours postdose signs or symptoms at the injection site were evaluated according to the Common Terminology Criteria for Adverse Events (CTCAE) v 5.0 criteria for ISR.AE Types: black upwards triangle (ISR grade 2), grey upwards triangle (ISR grade 1), grey point (dot) (non-ISR mild—did not meet the definition of an ISR). Each patient is represented by a line. Groups: dashed line (group B), solid lines (Groups C₁-C₃ with increasing thickness from C1 to C3). P and AD notations on the lines represent treatment arm of the subject (P is placebo, AD is active dose of HBVS-219). There were 2 Grade two ISRs and no Grade 3 or higher Grade ISRs.

The percentage of HBV flares recorded was 38% in the SD NUC-naïve Group B1 of patients. All flares occurred within the first 3 months of treatment in the NUC-naïve cohort. Of the 3 flares, all occurred after a single injection of HBVS-219.

There were no Serious Adverse Events in Groups B and C, no DLTs and no safety-related withdrawals. The average HBsAg reduction is:
>1 log (1.4 with SD 0.39) for Cohort C1 (light grey solid line in FIG. 4) at Day 112 and
>1.5 logs (1.8 with SD 0.57) for Cohort C2 (dark grey solid line in FIG. 4) at Day 112. C1 and C2 are statistically similar with a better response in C2 driven by a single subject, see FIG. 4.

The disclosure illustratively described herein suitably can be practised in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

According to the invention all embodiments for oligonucleotides for use in a method are also considered to be methods of treatment and/or for use in the manufacture of a medicament.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize or be able to ascertain using no more than routine experimentation, many equivalents to the specific embodiments of the invention described herein. Such equivalents are intended to be encompassed by the following claims.

### REFERENCES

Chang M. and Liam Y., Hepatitis B Flares in Chronic Hepatitis B: Pathogenesis, Natural Course and Management, J. HEPATOLOGY (2014) (Vol. 61) pp. 1407-17.
Chen C., et al., Long-term incidence and predictors of hepatitis B surface antigen loss after discontinuing nucleoside analogues in noncirrhotic chronic hepatitis B patients, CLIN. MICRO. INFECTION 24 (2018) 997-1003.
Chi H., et al., Flares During Long-Term Entecavir Therapy in Chronic Hepatitis B, J. GASTRO. HEPATOL. (2016) 31:1882-87.
Dusheiko G., Hepatitis B Surface Antigen Loss: Too Little, Too Late and the Challenge for the Future, GASTROENTEROLOGY, 156(3) P548-551 (February 2019).
Flink H J, et al., Flares in chronic hepatitis B patients induced by the host or the virus?Relation to treatment response during Peg-interferon a-2b therapy, GUT 2005; 54:1604-1609.
Fontana R. J. et al., Liver Safety Assessment in Clinical Trials of New Agents for Chronic Hepatitis B, J. VIROL HEPATOLOGY, (2020) 27:96-109.
Ghany M. G., et al., Serum Alanine Aminotransferase Flares in Chronic Hepatitis B Infection: The Good and the Bad, LANCET GASTROENTEROLOGY HEPATOLOGY, (2020) (5:406-17).
Höner Zu Siederdissen C, et al. Viral and host responses after stopping long-term nucleos (t)ide analogue therapy in HBeAg-negative chronic hepatitis B., J INFECT DIS. 2016; 214(10):1492-1497. doi: 10.1093/infdis/jiw412.
Marcellin P. et al., Adefovir dipivoxil for the treatment of hepatitis B e antigen-positive chronic hepatitis B, N Engl J Med 2003; 348:808-816.
Penna A., et al., Predominant T-helper 1 cytokine profile of hepatitis B virus nucleocapsid-specific T cells in acute self-limited hepatitis B, HEPATOLOGY 25: 1022-1027. (1997).
Schildgen O, et al., Variant of Hepatitis B virus with primary resistance to adefovir, N ENGL J MED 2006; 354:1807-1812.
Seeger C, Mason W S. Hepatitis B virus biology, MICROBIOL MOL BIOL REV. 2000; 64(1):51-68. doi: 10.1128/MMBR.64.1.51-68.2000.
Seto W K, et al., Treatment cessation of entecavir in Asian patients with hepatitis B e antigen negative chronic hepatitis B: a multicentre prospective study, GUT, 2015; 64(4):667-672. doi: 10.1136/gutjnl-2014-307237.
Stanaway J D, et al., The global burden of viral hepatitis from 1990 to 2013: findings from the Global Burden of Disease Study 2013, LANCET. 2016; 388(10049):1081-88. doi: 10.1016/S0140-6736(16)30579-7.
Tamaki N. et al., Hepatitis B surface antigen reduction by switching from long-term nucleoside/nucleotide analogue administration to pegylated interferon, J. VIRAL HEPAT., (2017) 4:672-78.
Thimme R., et al., CD8+ T Cells Mediate Viral Clearance and Disease Pathogenesis during Acute Hepatitis B Virus Infection, J. VIR., 77(1) 68-76 (2003).
Villeneuve J P., The natural history of chronic hepatitis B virus infection, J CLIN VIROL., 2005; 34 (Suppl 1):S139-S142.
Wong D., et al., ALT flares during nucleotide analogue therapy are associated with HBsAg loss in genotype A HBeAg-positive chronic hepatitis B, LIVER INTER. (2018) 38:1760-69.
Zoulim F., Assessment of treatment efficacy in HBV infection and disease, J HEPATOL 2006; 44:S95-S99.
Published Study—“Dose Response with the RNA Interference Therapy JNJ-3989 Combined with Nucleos(t)ide Analogue Treatment in Expanded Cohorts of Patients with Chronic Hepatitis B.”
HBeAg (Fried et al., 2008 Hepatology 47:428)
HbsAg (Moucari et al., 2009 Hepatology 49:1151)

1.-55. (canceled)

56. A method for treating hepatitis B or hepatitis B virus (HBV) infection in a human patient, the method comprising administering to the patient an oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
2′-fluoro modified nucleotides at positions 3, 8-10, 12, 13, and 17,
2′-O-methyl modified nucleotides at positions 1, 2, 4-7, 11, 14-16, 18-26, and 31-36, and a phosphorothioate linkage between the nucleotides at positions 1 and 2,
wherein each of the nucleotides of the -GAAA- sequence on the sense strand is conjugated to a monovalent GalNAc moiety; and
the antisense strand consists of a sequence as set forth in UUAUUGUGAGGAUUUUUGUCGG (SEQ ID NO: 2) and comprising
2′-fluoro modified nucleotides at positions 2, 3, 5, 7, 8, 10, 12, 14, 16, and 19,
2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
wherein the 4′-carbon of the sugar of the 5′-nucleotide of the antisense strand comprises a methoxy phosphonate (MOP),
or a pharmaceutically acceptable salt thereof,
the method comprising administering to the patient via subcutaneous route an initial dose of from about 0.1 mg/kg to about 12 mg/kg of the oligonucleotide, or an initial dose of from about 6 mg to about 800 mg of the oligonucleotide.

57. (canceled)

58. The method according to claim 56, wherein the hepatitis B or HBV infection is chronic hepatitis B or chronic HBV infection.

59. The method according to claim 56, wherein the initial dose is about 1.5 mg/kg, about 3 mg/kg or about 6 mg/kg, or about 100 mg, about 200 mg or about 400 mg.

60. (canceled)

61. The method according to claim 56, wherein the initial dose is a single dose or is the only dose administered.

62. The method according to claim 56, further comprising administering to the patient one or more subsequent doses of the oligonucleotide in an amount that is from about 0.1 mg/kg to about 12 mg/kg, or one or more subsequent doses of the oligonucleotide in an amount that is from about 6 mg to about 800 mg.

63.-65. (canceled)

66. The method according to claim 62, wherein the doses are separated in time from each other by at least about four weeks.

67. The method according to claim 62, wherein the doses are separated in time from each other by about four weeks and are administered over a period of about 48 weeks, about 24 weeks, about three months or about 12 weeks.

68. The method according to claim 62, wherein the period of time between each of the doses is independently selected from the group consisting of: about four weeks, about one month, about two months, about three months or about six months.

69. The method according to claim 62, wherein the period of time between each of the doses is as shown in any one of the regimens in Table 1.

70. The method according to claim 56, wherein the method comprises a treatment holiday, preferably of about three to about six months.

71. The method according to claim 56, wherein the patient is antiviral treatment naïve or the patient has not previously been treated with an antiviral therapy, preferably for a period of at least about six months.

72. (canceled)

73. The method according to claim 56, wherein the patient is: nucleot(s)ide analogue (NUC) suppressed, immune active, cirrhotic, immuno-tolerant, an inactive carrier, HBeAg positive, HBeAg negative, or HBV delta co-infection.

74. The method according to claim 56, wherein the oligonucleotide is administered as a monotherapy.

75. The method according to claim 56, wherein the method further comprises administering an effective amount of at least one additional therapeutic agent.

76. The method according to claim 75, wherein the additional therapeutic agent is an antiviral agent.

77. The method according to claim 76, wherein the antiviral agent is one or more of: interferon; ribavirin; an HBV RNA replication inhibitor; a second antisense oligomer; an HBV therapeutic vaccine; an HBV prophylactic vaccine; lamivudine (3TC); entecavir; tenofovir; telbivudine (LdT); adefovir; an HBV antibody therapy (monoclonal or polyclonal); an anti-PDL1/PD1 monoclonal antibody; an anti PD-L1 antisense oligonucleotide; a TLR7 agonist; a TLR8 agonist; and a CpAM.

78.-99. (canceled)

100. The method according to claim 75, wherein the oligonucleotide and the additional therapeutic agent are administered concomitantly or sequentially.

101. (canceled)

102. The method according to claim 75, wherein the method comprises a monotherapy lead-in phase, wherein one or more doses of the oligonucleotide are administered prior to the first dose of any additional therapeutic agent.

103. The method according to claim 56, wherein the patient has not previously been treated with an antiviral therapy, wherein the patient is administered an initial dose of the oligonucleotide of about 1.5 mg/kg, about 3 mg/kg or about 6 mg/kg, followed by three subsequent doses of the oligonucleotide each of about 1.5 mg/kg, about 3 mg/kg or about 6 mg/kg, wherein the doses are separated in time from each other by a period of about four weeks.

104. The method according to claim 56, wherein the patient has not previously been treated with an antiviral therapy, wherein the method consists of the administration of one dose of the oligonucleotide in an amount of about 1.5 mg/kg, about 3 mg/kg or about 6 mg/kg.

105. The method according to claim 56, wherein the patient has not previously been treated with an antiviral therapy, wherein the method comprises the administration of a single dose of the oligonucleotide in an amount of about 1.5 mg/kg, about 3 mg/kg or about 6 mg/kg.

106. The method according to claim 56, wherein the oligonucleotide comprises a sense strand forming a duplex region with an antisense strand, wherein:
the sense strand consists of a sequence as set forth in GACAAAAAUCCUCACAAUAAGCAGCCGAAAGGCUGC (SEQ ID NO: 1) and comprising
2′-fluoro modified nucleotides at positions 3, 8-10, 12, 13, and 17,
2′-O-methyl modified nucleotides at positions 1, 2, 4-7, 11, 14-16, 18-26, and 31-36, and one phosphorothioate linkage between the nucleotides at positions 1 and 2,
wherein each of the nucleotides of the -GAAA- sequence on the sense strand is conjugated to a monovalent GalNAc moiety; wherein the -GAAA- sequence comprises the structure. 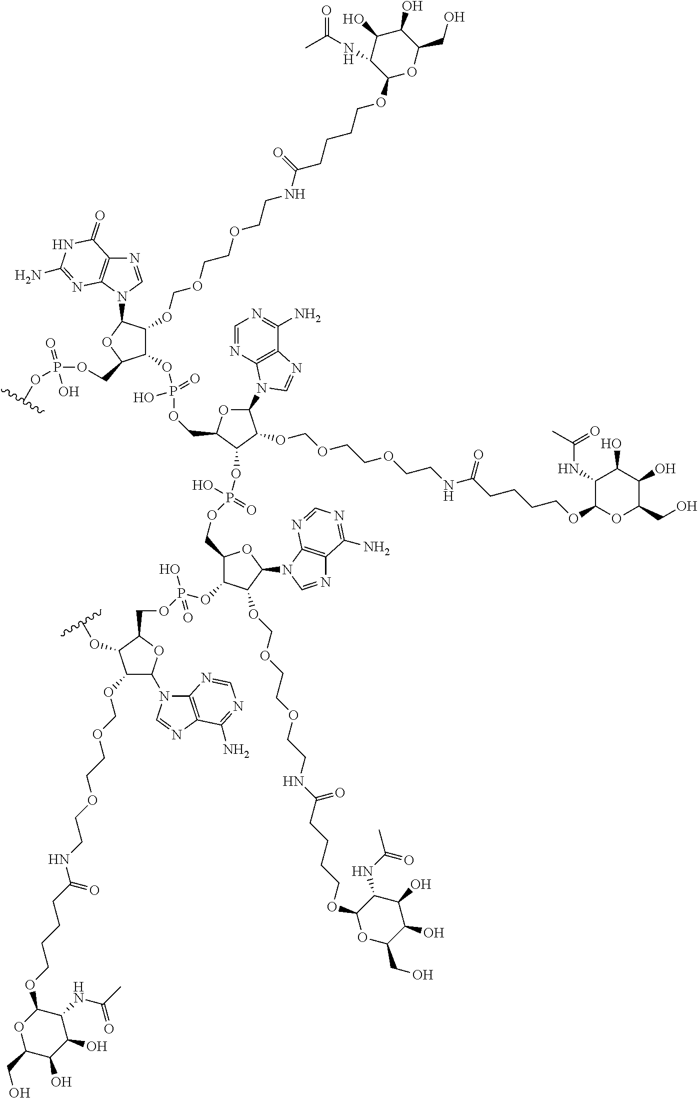 [SMILES: *O[C@@H]1[C@@H](COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(N)ncnc43)O[C@@H]2COP(=O)(O)O[C@H]2[C@@H](OCOCCOCCNC(=O)CCCCO[C@@H]3O[C@H](CO)[C@H](O)[C@H](O)[C@H]3NC(C)=O)[C@H](n3cnc4c(=O)[nH]c(N)nc43)O[C@@H]2COP(=O)(O)O*)OC(n2cnc3c(N)ncnc32)[C@@H]1OCOCCOCCNC(=O)CCCCO[C@@H]1O[C@H](CO)[C@H](O)[C@H](O)[C@H]1NC(C)=O]
and
the antisense strand consists of a sequence as set forth in UUAUUGUGAGGAUUUUUGUCGG (SEQ ID NO: 2) and comprising
2′-fluoro modified nucleotides at positions 2, 3, 5, 7, 8, 10, 12, 14, 16, and 19,
2′-O-methyl modified nucleotides at positions 1, 4, 6, 9, 11, 13, 15, 17, 18, and 20-22, and five phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
wherein the 5′-nucleotide of the antisense strand has the following structure: 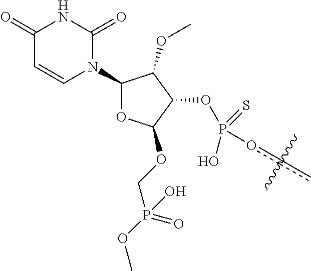
or a pharmaceutically acceptable salt thereof.

107. The method according to claim 56, wherein the oligonucleotide is in the form of a pharmaceutically acceptable salt, preferably a sodium salt or a potassium salt.

108. The method according to claim 107, wherein the pharmaceutically acceptable salt of the oligonucleotide is as shown in FIG. 2a or FIG. 2b.

109. A method for treating hepatitis B or hepatitis B virus (HBV) infection in a human patient, the method comprising administering to the patient a pharmaceutical composition comprising the oligonucleotide or pharmaceutically acceptable salt thereof as defined in claim 56 and a pharmaceutically acceptable solvent, carrier, excipient, diluent or adjuvant, the method comprising administering to the patient via subcutaneous route an initial dose of from about 0.1 mg/kg to about 12 mg/kg of the oligonucleotide.

110. The method according to claim 109, wherein the pharmaceutically acceptable solvent, carrier, excipient, diluent or adjuvant is phosphate buffered saline.

111.-112. (canceled)


SEQUENCE LISTING (79 sequences)

SEQ ID NO: 1 - RNA, 36 nt, synthetic construct
  misc_feature 1..36: note = synthetic polynucleotide
  source 1..36: mol_type = other RNA
gacaaaaatcctcacaataagcagccgaaaggctgc

SEQ ID NO: 2 - RNA, 22 nt, synthetic construct
  misc_feature 1..22: note = synthetic polynucleotide
  source 1..22: mol_type = other RNA
ttattgtgaggatttttgtcgg

SEQ ID NO: 3 - DNA, 19 bp, synthetic construct
  misc_feature 1..19: note = oligonucleotide motif
  source 1..19: mol_type = other DNA
cacctatttaacatcagac

SEQ ID NO: 4 - protein, 5 aa, synthetic construct
  REGION 1..5: note = Bc1.187 CDR-H1
NYGMQ

SEQ ID NO: 5 - protein, 17 aa, synthetic construct
  REGION 1..17: note = Bc1.187 CDR-H2
IIWADGTKQYYGDSVKG

SEQ ID NO: 6 - protein, 11 aa, synthetic construct
  REGION 1..11: note = Bc1.187 CDR-H3
DGLYASAPNDV

SEQ ID NO: 7 - protein, 11 aa, synthetic construct
  REGION 1..11: note = Bc1.187 CDR-L1
RASQRISTYLN

SEQ ID NO: 8 - protein, 7 aa, synthetic construct
  REGION 1..7: note = Bc1.187-CDR-L2
GASSLQS

SEQ ID NO: 9 - protein, 9 aa, synthetic construct
  REGION 1..9: note = Bc1.187 CDR-L3
QQTYTLPPN

SEQ ID NO: 10 - protein, 30 aa, synthetic construct
  REGION 1..30: note = Bc1.187 H-FW1
QVQLVESGGGVVQPGRSLRLSCEASGFTFS

SEQ ID NO: 11 - protein, 14 aa, synthetic construct
  REGION 1..14: note = Bc1.187 H-FW2
WVRQAPGKGLEWVA

SEQ ID NO: 12 - protein, 31 aa, synthetic construct
  REGION 1..31: note = Bc1.187 H-FW3
FTISRDNFKNTLYLQMNSLRGEDTAMYFCAR

SEQ ID NO: 13 - protein, 11 aa, synthetic construct
  REGION 1..11: note = Bc1.187 H-FW4
WGQGTLVTVSS

SEQ ID NO: 14 - protein, 23 aa, synthetic construct
  REGION 1..23: note = Bc1.187 L-FW1
DIQMTQSPSSLSAYVGDRVTITC

SEQ ID NO: 15 - protein, 15 aa, synthetic construct
  REGION 1..15: note = Bc1.187 L-FW2
WYHQRPGKSPSLLIY

SEQ ID NO: 16 - protein, 32 aa, synthetic construct
  REGION 1..32: note = Bc1.187 L-FW3
GVPSRFSASASGTDFTLTISSLRPEDLGTYYC

SEQ ID NO: 17 - protein, 10 aa, synthetic construct
  REGION 1..10: note = Bc1.187 L-FW4
SGGGTKVEIK

SEQ ID NO: 18 - protein, 107 aa, synthetic construct
  REGION 1..107: note = Bc1.187 VL
DIQMTQSPSSLSAYVGDRVTITCRASQRISTYLNWYHQRPGKSPSLLIYGASSLQSGVPSRFSASASGTDFTLTISSLRPEDLGTYYCQQTYTLPPNSGGGTKVEIK

SEQ ID NO: 19 - protein, 120 aa, synthetic construct
  REGION 1..120: note = Bc1.187 VH
QVQLVESGGGVVQPGRSLRLSCEASGFTFSNYGMQWVRQAPGKGLEWVAIIWADGTKQYYGDSVKGRFTISRDNFKNTLYLQMNSLRGEDTAMYFCARDGLYASAPNDVWGQGTLVTVSS

SEQ ID NO: 20 - protein, 214 aa, synthetic construct
  REGION 1..214: note = Bc1.187 full length light chain
DIQMTQSPSSLSAYVGDRVTITCRASQRISTYLNWYHQRPGKSPSLLIYGASSLQSGVPSRFSASASGTDFTLTISSLRPEDLGTYYCQQTYTLPPNSGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 449 aa, synthetic construct
  REGION 1..449: note = Bc1.187 full length heavy chain
QVQLVESGGGVVQPGRSLRLSCEASGFTFSNYGMQWVRQAPGKGLEWVAIIWADGTKQYYGDSVKGRFTISRDNFKNTLYLQMNSLRGEDTAMYFCARDGLYASAPNDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, synthetic construct
  REGION 1..5: note = Bc3.106 CDR-H1
SYAMS

SEQ ID NO: 23 - protein, 17 aa, synthetic construct
  REGION 1..17: note = Bc3.106 CDR-H2
AFSGTGGSTYYADSVKG

SEQ ID NO: 24 - protein, 19 aa, synthetic construct
  REGION 1..19: note = Bc3.106 CDR-H3
DPGHTSNWRDNYQYYQMDV

SEQ ID NO: 25 - protein, 11 aa, synthetic construct
  REGION 1..11: note = Bc3.106 CDR-L1
RASQGIRNDLG

SEQ ID NO: 26 - protein, 7 aa, synthetic construct
  REGION 1..7: note = Bc3.106-CDR-L2
AASSLQS

SEQ ID NO: 27 - protein, 9 aa, synthetic construct
  REGION 1..9: note = Bc3.106 CDR-L3
LQHNSYPRT

SEQ ID NO: 28 - protein, 30 aa, synthetic construct
  REGION 1..30: note = Bc3.106 H-FW1
EVQLLESGGGLVQPGGSLRLSCTASGFTFG

SEQ ID NO: 29 - protein, 14 aa, synthetic construct
  REGION 1..14: note = Bc3.106 H-FW2
WVRQAPGKGLKWVS

SEQ ID NO: 30 - protein, 32 aa, synthetic construct
  REGION 1..32: note = Bc3.106 H-FW3
RFTISRDNSKNTLYLQMNNLRAEDTAVYFCAK

SEQ ID NO: 31 - protein, 11 aa, synthetic construct
  REGION 1..11: note = Bc3.106 H-FW4
WGQGTTVTVSS

SEQ ID NO: 32 - protein, 23 aa, synthetic construct
  REGION 1..23: note = Bc3.106 L-FW1
DIQMTQSPSSLSASVGDRVTITC

SEQ ID NO: 33 - protein, 15 aa, synthetic construct
  REGION 1..15: note = Bc3.106 L-FW2
WYQQKPGKAPKRLIY

SEQ ID NO: 34 - protein, 32 aa, synthetic construct
  REGION 1..32: note = Bc3.106 L-FW3
GVPSRFSGSGSGTEFTLTISSLQPEDFATYYC

SEQ ID NO: 35 - protein, 10 aa, synthetic construct
  REGION 1..10: note = Bc3.106 L-FW4
FGQGTKVEIK

SEQ ID NO: 36 - protein, 107 aa, synthetic construct
  REGION 1..107: note = Bc3.106 VL
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 37 - protein, 128 aa, synthetic construct
  REGION 1..128: note = Bc3.106 VH
EVQLLESGGGLVQPGGSLRLSCTASGFTFGSYAMSWVRQAPGKGLKWVSAFSGTGGSTYYADSVKGRFTISRDNSKNTLYLQMNNLRAEDTAVYFCAKDPGHTSNWRDNYQYYQMDVWGQGTTVTVSS

SEQ ID NO: 38 - protein, 214 aa, synthetic construct
  REGION 1..214: note = Bc3.106 full length light chain
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 457 aa, synthetic construct
  REGION 1..457: note = Bc3.106 full length heavy chain
EVQLLESGGGLVQPGGSLRLSCTASGFTFGSYAMSWVRQAPGKGLKWVSAFSGTGGSTYYADSVKGRFTISRDNSKNTLYLQMNNLRAEDTAVYFCAKDPGHTSNWRDNYQYYQMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 5 aa, synthetic construct
  REGION 1..5: note = Bv4.115 CDR-H1
NYHIH

SEQ ID NO: 41 - protein, 17 aa, synthetic construct
  REGION 1..17: note = Bv4.115 CDR-H2
IINPRRLSTAYAPKFQG

SEQ ID NO: 42 - protein, 12 aa, synthetic construct
  REGION 1..12: note = Bv4.115 CDR-H3
DAGDDTSGPFDS

SEQ ID NO: 43 - protein, 11 aa, synthetic construct
  REGION 1..11: note = Bv4.115 CDR-L1
RASQSINTWLA

SEQ ID NO: 44 - protein, 7 aa, synthetic construct
  REGION 1..7: note = Bv4.115-CDR-L2
KASSLES

SEQ ID NO: 45 - protein, 7 aa, synthetic construct
  REGION 1..7: note = Bv4.115 CDR-L3
QQYNTFS

SEQ ID NO: 46 - protein, 30 aa, synthetic construct
  REGION 1..30: note = Bv4.115 H-FW1
QVQLVQSGAEVKKPGSSVKVSCRSSGYRFT

SEQ ID NO: 47 - protein, 14 aa, synthetic construct
  REGION 1..14: note = Bv4.115 H-FW2
WVRQAPGQGLEWVG

SEQ ID NO: 48 - protein, 32 aa, synthetic construct
  REGION 1..32: note = Bv4.115 H-FW3
RVTMTRDTSTSTVYMELSSLRSDDTAVYYCAR

SEQ ID NO: 49 - protein, 11 aa, synthetic construct
  REGION 1..11: note = Bv4.115 H-FW4
WGQGTLVTVSS

SEQ ID NO: 50 - protein, 23 aa, synthetic construct
  REGION 1..23: note = Bv4.115 L-FW1
DIQMTQSPSTLSASVGDRVTITC

SEQ ID NO: 51 - protein, 15 aa, synthetic construct
  REGION 1..15: note = Bv4.115 L-FW2
WYQQKPGKAPKLLIS

SEQ ID NO: 52 - protein, 32 aa, synthetic construct
  REGION 1..32: note = Bv4.115 L-FW3
GVPSRFSGSGSGTEFTLSISSLQPDDFATYYC

SEQ ID NO: 53 - protein, 10 aa, synthetic construct
  REGION 1..10: note = Bv4.115 L-FW4
FGQGTKLEIK

SEQ ID NO: 54 - protein, 105 aa, synthetic construct
  REGION 1..105: note = Bv4.115 VL
DIQMTQSPSTLSASVGDRVTITCRASQSINTWLAWYQQKPGKAPKLLISKASSLESGVPSRFSGSGSGTEFTLSISSLQPDDFATYYCQQYNTFSFGQGTKLEIK

SEQ ID NO: 55 - protein, 121 aa, synthetic construct
  REGION 1..121: note = Bv4.115 VH
QVQLVQSGAEVKKPGSSVKVSCRSSGYRFTNYHIHWVRQAPGQGLEWVGIINPRRLSTAYAPKFQGRVTMTRDTSTSTVYMELSSLRSDDTAVYYCARDAGDDTSGPFDSWGQGTLVTVSS

SEQ ID NO: 56 - protein, 212 aa, synthetic construct
  REGION 1..212: note = Bv4.115 full length light chain
DIQMTQSPSTLSASVGDRVTITCRASQSINTWLAWYQQKPGKAPKLLISKASSLESGVPSRFSGSGSGTEFTLSISSLQPDDFATYYCQQYNTFSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 450 aa, synthetic construct
  REGION 1..450: note = Bv4.115 full length heavy chain
QVQLVQSGAEVKKPGSSVKVSCRSSGYRFTNYHIHWVRQAPGQGLEWVGIINPRRLSTAYAPKFQGRVTMTRDTSTSTVYMELSSLRSDDTAVYYCARDAGDDTSGPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 6 aa, synthetic construct
  REGION 1..6: note = Bc8.159 CDR-H1
TNNWWS

SEQ ID NO: 59 - protein, 16 aa, synthetic construct
  REGION 1..16: note = Bc8.159 CDR-H2
EIHHIGSTNYNPSLKS

SEQ ID NO: 60 - protein, 14 aa, synthetic construct
  REGION 1..14: note = Bc8.159 CDR-H3
GRLGITRDRYYFDS

SEQ ID NO: 61 - protein, 11 aa, synthetic construct
  REGION 1..11: note = Bc8.159 CDR-L1
QASQDISNYLN

SEQ ID NO: 62 - protein, 7 aa, synthetic construct
  REGION 1..7: note = Bc8.159-CDR-L2
DTSSLER

SEQ ID NO: 63 - protein, 9 aa, synthetic construct
  REGION 1..9: note = Bc8.159 CDR-L3
QQYYNLPHT

SEQ ID NO: 64 - protein, 30 aa, synthetic construct
  REGION 1..30: note = Bc8.159 H-FW1
QVQLQESGPGLVKPSGTLSLTCAVSGGTIR

SEQ ID NO: 65 - protein, 14 aa, synthetic construct
  REGION 1..14: note = Bc8.159 H-FW2
WVRQPPGKGLEWIG

SEQ ID NO: 66 - protein, 32 aa, synthetic construct
  REGION 1..32: note = Bc8.159 H-FW3
QVTISVDKSKNQFSLNLSSVTAADTALYYCVR

SEQ ID NO: 67 - protein, 11 aa, synthetic construct
  REGION 1..11: note = Bc8.159 H-FW4
WGRGTLVTVSS

SEQ ID NO: 68 - protein, 23 aa, synthetic construct
  REGION 1..23: note = Bc8.159 L-FW1
DIQMTQSPSPLSVSVGDRVTITC

SEQ ID NO: 69 - protein, 15 aa, synthetic construct
  REGION 1..15: note = Bc8.159 L-FW2
WYQQKPGQAPKLLIY

SEQ ID NO: 70 - protein, 32 aa, synthetic construct
  REGION 1..32: note = Bc8.159 L-FW3
GVPSRFSGSGSGTDFTLTISSLQPEDIATYHC

SEQ ID NO: 71 - protein, 10 aa, synthetic construct
  REGION 1..10: note = Bc8.159 L-FW4
FGQGTKLEIK

SEQ ID NO: 72 - protein, 107 aa, synthetic construct
  REGION 1..107: note = Bc8.159 VL
DIQMTQSPSPLSVSVGDRVTITCQASQDISNYLNWYQQKPGQAPKLLIYDTSSLERGVPSRFSGSGSGTDFTLTISSLQPEDIATYHCQQYYNLPHTFGQGTKLEIK

SEQ ID NO: 73 - protein, 123 aa, synthetic construct
  REGION 1..123: note = Bc8.159 VH
QVQLQESGPGLVKPSGTLSLTCAVSGGTIRTNNWWSWVRQPPGKGLEWIGEIHHIGSTNYNPSLKSQVTISVDKSKNQFSLNLSSVTAADTALYYCVRGRLGITRDRYYFDSWGRGTLVTVSS

SEQ ID NO: 74 - protein, 214 aa, synthetic construct
  REGION 1..214: note = Bc8.159 full length light chain
DIQMTQSPSPLSVSVGDRVTITCQASQDISNYLNWYQQKPGQAPKLLIYDTSSLERGVPSRFSGSGSGTDFTLTISSLQPEDIATYHCQQYYNLPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 452 aa, synthetic construct
  REGION 1..452: note = Bc8.159 full length heavy chain
QVQLQESGPGLVKPSGTLSLTCAVSGGTIRTNNWWSWVRQPPGKGLEWIGEIHHIGSTNYNPSLKSQVTISVDKSKNQFSLNLSSVTAADTALYYCVRGRLGITRDRYYFDSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 450 aa, synthetic construct
  REGION 1..450: note = Bc1.187 full length heavy chain (containing modifications)
QVQLVESGGGVVQPGRSLRLSCEASGFTFSNYGMQWVRQAPGKGLEWVAIIWADGTKQYYGDSVKGRFTISRDNFKNTLYLQMNSLRGEDTAMYFCARDGLYASAPNDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 458 aa, synthetic construct
  REGION 1..458: note = Bc3.106 full length heavy chain (containing modifications)
EVQLLESGGGLVQPGGSLRLSCTASGFTFGSYAMSWVRQAPGKGLKWVSAFSGTGGSTYYADSVKGRFTISRDNSKNTLYLQMNNLRAEDTAVYFCAKDPGHTSNWRDNYQYYQMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 451 aa, synthetic construct
  REGION 1..451: note = Bv4.115 full length heavy chain (containing modifications)
QVQLVQSGAEVKKPGSSVKVSCRSSGYRFTNYHIHWVRQAPGQGLEWVGIINPRRLSTAYAPKFQGRVTMTRDTSTSTVYMELSSLRSDDTAVYYCARDAGDDTSGPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 453 aa, synthetic construct
  REGION 1..453: note = Bc8.159 full length heavy chain (containing modifications)
QVQLQESGPGLVKPSGTLSLTCAVSGGTIRTNNWWSWVRQPPGKGLEWIGEIHHIGSTNYNPSLKSQVTISVDKSKNQFSLNLSSVTAADTALYYCVRGRLGITRDRYYFDSWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK